(19) United States
(12) Patent Application Publication
(10) Pub. No.: US 2022/0205049 A1
(43) Pub. Date: Jun. 30, 2022

(54) METHODS OF DETECTING AND PREDICTING BREAST CANCER

(71) Applicant: Martin Widschwendter, (US)

(72) Inventor: Martin Widschwendter, (US)

(21) Appl. No.: 17/596,594

(22) PCT Filed: Jun. 12, 2020
(86) PCT No.: PCT/GB2020/051421

§ 371 Date: Dec. 14, 2021

(30) Foreign Application Priority Data
Jun. 14, 2019 (GB) 1908591.9

(51) Int. Cl. C12Q 1/6886; C12Q 1/6827; C12Q 1/6837; G01N 33/50; A61K 31/567; C07K 16/28; A61P 35/00

(52) U.S. Cl. CPC C12Q 1/6886; C12Q 1/6827; C12Q 1/6837; G01N 33/5091; C12Q 2600/154; C07K 16/2875; A61P 35/00; C12Q 2600/118; A61K 31/567

(57) ABSTRACT

The present invention relates to assays for predicting the presence or development of breast cancer in an individual, by determining the methylation status of certain CpGs in DNA from the individual, deriving an index value based on the methylation status of the certain CpGs, and predicting the development of breast cancer in the individual based on the breast cancer index value.

### SEQUENCE LISTING

The instant application contains a Sequence Listing which has been submitted electronically in ASCII format and is hereby incorporated by reference in its entirety. Said ASCII copy, created on Jun. 11, 2020, is named N416449WO_SL.txt and is 21,495,630 bytes in size.

### FIELD OF THE INVENTION

The present invention relates to assays for predicting the presence or development of breast cancer in an individual, by determining the methylation status of certain CpGs in DNA from the individual, deriving an index value based on the methylation status of the certain CpGs, and predicting the development of breast cancer in the individual based on the breast cancer index value.

The invention also relates to methods for monitoring the risk of an individual harbouring breast cancer or of breast cancer development.

The invention also relates to methods of treating breast cancer comprising predicting the presence or development of breast cancer in an individual by the assays described herein, stratification of the individual according to their risk, and administering one or more treatments to the individual based on their risk.

The invention also relates to methylation-discriminatory arrays comprising probes directed to the CpGs defined herein.

### BACKGROUND TO THE INVENTION

Breast cancer is by far the most common cancer in females in general, and a leading cause of death in young women¹. To date, the identification of individuals with primary cancer is achieved by assessing evidence directly from the tumour (e.g. imaging or detection of cancer cell products released into the system^{3,4}). Currently available early detection strategies, such as mammography screening, suffer from low performance in young women, over-diagnosis, and decreasing attendance rates, and its benefit on mortality rates has recently been questioned⁵. Developing models which allow for a stratified breast cancer early detection and prevention strategy have proven to be challenging, and the best predictive models combining epidemiological risk factors, single nucleotide polymorphisms (SNPs) and mammographic density have only led to a Receiver Operator Characteristic (ROC) Area Under the Curve (AUC) of 0.68⁶.

In contrast, cervical cancer screening (i.e. assessing cervical smear samples) has reduced the incidence and mortality from cervical cancer by more than 50%⁷. The fact that clinician- and self-collected samples show similar performance in detecting relevant cervical lesions⁸ is likely to further increase attendance rates.

Epigenetic (i.e. DNAme) changes have been identified in normal breast tissue adjacent to breast cancers⁹ and could potentially serve as a surrogate for both genetic and non-genetic factors including lifestyle, reproductive and environmental exposures contributing to breast cancer development¹⁰. A number of proof of principle studies, so far exclusively performed in blood, have demonstrated that certain DNAme (DNA methylation) changes are associated with breast cancer predisposition¹¹⁻¹⁶. Sample heterogeneity and the choice of surrogate tissue are deemed to be among the most important factors impeding clinical implementation¹⁷. Thus, there remains an urgent need to identify new methylation-based CpG loci, and sets of loci, which may act as biomarkers or which may contribute to the understanding of methylation patterns or signatures useful in the field of cancer.

### SUMMARY OF THE INVENTION

The current inventors set out to understand whether DNAme patterns may be associated with the development of breast cancer. The inventors have shown that DNAme signatures of samples derived from breast tissue and, remarkably, from samples derived from an anatomical site other than the breast identify women with breast cancer. The inventors have determined that the DNAme signatures can be characterized according to a breast cancer index value which can be used to stratify individuals according to their risk of harbouring breast cancer or according to their risk of breast cancer development. Moreover, the DNAme signatures were shown to be changeable in response to breast cancer therapies, thus indicating the dynamic nature of the identified predictive DNAme signature, and thus surprisingly indicating that the DNAme signatures of the invention can be used to monitor risk associations with breast cancer.

Thus the invention provides an assay for predicting the presence or development of breast cancer in an individual, the assay comprising:
a. providing a sample which has been taken from the individual;
b. determining in DNA in the sample the methylation status of each CpG in a set of test CpGs selected from the panel of CpGs identified at nucleotide positions 61 to 62 in SEQ ID NOs 1 to 40,753;
c. deriving a breast cancer index value based on the methylation status of the test CpGs; and
d. predicting the presence or development of breast cancer in the individual based on the breast cancer index value;
wherein the assay is characterised as having an area under the curve (AUC) of 0.6 or more as determined by receiver operating characteristics (ROC).

The assay of the invention may be described above and additionally wherein the sample from the individual is a sample from:
a. an anatomical site other than the breast, such as a cervical, vaginal, or preferably a cervicovaginal smear; or
b. the breast.

In any of the assays described herein, sample may particularly be derived from the cervix, the vagina, the buccal area, blood and/or urine. The sample is preferably a cervical liquid-based cytology sample, and more preferably a cervical smear sample.

The assay of the invention may be performed above and additionally wherein the DNA from the sample is derived from an organ comprising epithelial cells.

The assay of the invention may be performed above and additionally wherein the cancer is ductal carcinoma in situ; an invasive ductal carcinoma such as tubular type invasive ductal carcinoma (IDC), medullary type IDC, mucinous type IDC, papillary type IDC, cribriform type IDC; invasive lobular carcinoma, inflammatory breast cancer, lobular carcinoma in situ, male breast cancer, luminal A breast cancer, luminal B breast cancer, triple-negative/basal-like breast cancer, HER2-enriched breast cancer, normal-like breast cancer, Paget's Disease of the nipple, Phyllodes tumours of the breast, or metastatic breast cancer.

The assay of the invention may be performed above and additionally wherein the step of determining the methylation status of each CpG in the set of test CpGs comprises determining methylation β-values for each one of the test CpGs.

The assay of the invention may be performed above and additionally wherein the step of deriving the breast cancer index value based on the methylation status of the test CpGs comprises:
a. providing a methylation β-value data set comprising the methylation (3-values for each test CpG;
b. estimating an immune cell DNA fraction within the DNA provided from the sample; and
c. applying an algorithm to the methylation β-value data set that controls for the immune cell DNA fraction within the DNA provided from the sample to obtain the breast cancer index value.

The assay of the invention may be performed above and additionally wherein the breast cancer index value is termed Women's risk Identification for Breast Cancer Index (WID-BC-Index) and is calculated by an algorithm according to the following formula:

$$WID - BC - Index = {\sum }_{i = 1}^{n} \frac{μ - ( {a}_{i} + {b}_{i} ρ ) {β}_{i}}{σ}$$

wherein:
e. ρ∈[0,1] is the immune cell DNA fraction for the sample;
f. β₁, . . . , βₙ are methylation beta-values (between 0 and 1);
g. a₁, . . . , aₙ and b₁, bₙ are real valued coefficients;
h. μ and σ are real valued parameters used to scale the index; and
i. n refers to the number of CpGs in the set of test CpGs

The assay of the invention may be performed above and additionally wherein the set of CpGs comprises at least 500 CpGs selected from the CpGs identified at nucleotide positions 61 to 62 in SEQ ID NOs 1 to 40,753, preferably wherein the assay is characterised as having an AUC of at least 0.73.

The assay of the invention may be performed above and additionally wherein the set of CpGs comprises at least the CpGs identified in SEQ ID NOs 1 to 500 and identified at nucleotide positions 61 to 62.

The assay of the invention may be performed above and additionally wherein the set of CpGs comprises at least 1000 CpGs selected from the CpGs identified at nucleotide positions 61 to 62 in SEQ ID NOs 1 to 40,753, preferably wherein the assay is characterised as having an AUC of at least 0.77.

The assay of the invention may be performed above and additionally wherein the set of CpGs comprises at least the CpGs identified in SEQ ID NOs 1 to 1000 and identified at nucleotide positions 61 to 62.

The assay of the invention may be performed above and additionally wherein the set of CpGs comprises at least 2000 CpGs selected from the CpGs identified at nucleotide positions 61 to 62 in SEQ ID NOs 1 to 40,753, preferably wherein the assay is characterised as having an AUC of at least 0.81.

The assay of the invention may be performed above and additionally wherein the set of CpGs comprises at least the CpGs identified in SEQ ID NOs 1 to 2000 and identified at nucleotide positions 61 to 62.

The assay of the invention may be performed above and additionally wherein the set of CpGs comprises at least 10,000 CpGs selected from the CpGs identified at nucleotide positions 61 to 62 in SEQ ID NOs 1 to 40,753, preferably wherein the assay is characterised as having an AUC of at least 0.84.

The assay of the invention may be performed above and additionally wherein the set of CpGs comprises at least the CpGs identified in SEQ ID NOs 1 to 10,000 and identified at nucleotide positions 61 to 62.

The assay of the invention may be performed above and additionally wherein the set of CpGs comprises at least the 40,753 CpGs identified in SEQ ID NOs 1 to 40,753 and identified at nucleotide positions 61 to 62, and further wherein the assay is characterised as having an AUC of at least 0.85.

The assay of the invention may be performed above and additionally wherein the predicting of the presence or development of breast cancer in an individual is based on the WID-BC-Index.

The assay of the invention may be performed above and additionally wherein when the WID-BC-Index for the individual is about −0.235 or more, the individual is classified as having at least a low risk of harbouring breast cancer or a low risk of breast cancer development.

The assay of the invention may be performed above and additionally wherein the set of CpGs comprises at least 500 of the CpGs defined by SEQ ID NOs 1 to 40,753 and identified at nucleotide positions 61 to 62, and wherein the sensitivity is at least 58% and the specificity is at least 44%.

The assay of the invention may be performed above and additionally wherein the set of CpGs comprises at least the CpGs defined by SEQ ID NOs 1 to 500 and identified at nucleotide positions 61 to 62, and wherein the sensitivity is at least 85% and specificity is at least 52%.

The assay of the invention may be performed above and additionally wherein the set of CpGs comprises at least the CpGs defined by SEQ ID NOs 1 to 1000 and identified at nucleotide positions 61 to 62, and wherein the sensitivity is at least 88% and specificity is at least 49%.

The assay of the invention may be performed above and wherein the set of CpGs comprises at least the CpGs defined by SEQ ID NOs 1 to 2000 and identified at nucleotide positions 61 to 62, and wherein the sensitivity is at least 94% and specificity is at least 51%.

The assay of the invention may be performed above and additionally wherein when the WID-BC-Index for the individual is about 0.090 or more, the individual is classified as having at least a moderate risk of harbouring breast cancer or a moderate risk of breast cancer development.

The assay of the invention may be performed above and additionally wherein the set of CpGs comprises at least 500 of the CpGs defined by SEQ ID NOs 1 to 40,753 and identified at nucleotide positions 61 to 62, and wherein the sensitivity is at least 35% and the specificity is at least 63%.

The assay of the invention may be performed above and additionally wherein the set of CpGs comprises at least the CpGs defined by SEQ ID NOs 1 to 500 and identified at nucleotide positions 61 to 62, and wherein the sensitivity is at least 63% and specificity is at least 69%.

The assay of the invention may be performed above and additionally wherein the set of CpGs comprises at least the CpGs defined by SEQ ID NOs 1 to 1000 and identified at nucleotide positions 61 to 62, and wherein the sensitivity is at least 68% and specificity is at least 73%.

The assay of the invention may be performed above and additionally wherein the set of CpGs comprises at least the CpGs defined by SEQ ID NOs 1 to 2000 and identified at nucleotide positions 61 to 62, and wherein the sensitivity is at least 69% and specificity is at least 78%.

The assay of the invention may be performed above and additionally wherein when the WID-BC-Index for the individual is about 0.587 or more, the individual is classified as having a high risk of harbouring breast cancer or a high risk of breast cancer development.

The assay of the invention may be performed above and additionally wherein the set of CpGs comprises at least 500 of the CpGs defined by SEQ ID NOs 1 to 40,753 and identified at nucleotide positions 61 to 62, and wherein the sensitivity is at least 24% and the specificity is at least 84%.

The assay of the invention may be performed above and additionally wherein the set of CpGs comprises at least the CpGs defined by SEQ ID NOs 1 to 500 and identified at nucleotide positions 61 to 62, and wherein the sensitivity is at least 26% and specificity is at least 93%.

The assay of the invention may be performed above and additionally wherein the set of CpGs comprises at least the CpGs defined by SEQ ID NOs 1 to 1000 and identified at nucleotide positions 61 to 62, and wherein the sensitivity is at least 29% and specificity is at least 95%.

The assay of the invention may be performed above and additionally wherein the set of CpGs comprises at least the CpGs defined by SEQ ID NOs 1 to 2000 and identified at nucleotide positions 61 to 62, and wherein the sensitivity is at least 33% and specificity is at least 94%.

The assay of the invention may be performed above and additionally wherein the step of determining the methylation status of each CpG in the set of test CpGs comprises bisulphite converting the DNA.

The assay of the invention may be performed above and additionally wherein the step of determining the methylation status of each CpG in the set of test CpGs comprises:
a. performing a sequencing step to determine the sequence of each CpG;

b. hybridising DNA to an array comprising probes capable of discriminating between methylated and non-methylated forms of the CpGs and applying a detection system to the array so as to determine the methylation status or β-value of each CpG; or
c. performing a PCR step using methylation-specific primers, wherein the methylation status of the CpG is determined by the presence or absence of a PCR product.

The assay of the invention may be performed above and additionally wherein the assay further comprises:
a. determining in the sample from the individual the proportion of epithelial cells;
b. determining in the sample from the individual the proportion of fat cells; and/or
c. determining in the sample from the individual differentiation characteristics of non-fat cells.

The assay of the invention may be performed above and additionally wherein determining the proportion of epithelial and/or fat cells and/or determining differentiation characteristics of non-fat cells comprises performing a method comprising:
a. gene expression profiling;
b. non-coding RNA-profiling;
c. epigenome profiling;
d. DNA methylation profiling;
e. deriving a WID-BC-Index; and/or
f. immunohistochemistry; and
arriving at a determination based on the results of the method.

The invention also provides an array capable of discriminating between methylated and non-methylated forms of CpGs; the array comprising oligonucleotide probes specific for a methylated form of each CpG in a CpG panel and oligonucleotide probes specific for a non-methylated form of each CpG in the panel; wherein the panel consists of at least 500 CpGs selected from the CpGs identified in SEQ ID NOs 1 to 40,753.

The array of the invention may be as above and additionally provided that the array is not an Infinium MethylationEPIC BeadChip array or an Infinium HumanMethylation450, and/or provided that the number of CpG-specific oligonucleotide probes of the array is 482,000 or less, 480,000 or less, 450,000 or less, 440,000 or less, 430,000 or less, 420,000 or less, 410,000 or less, or 400,000 or less.

The array of the invention may be as above and additionally wherein the panel comprises any set of CpGs defined in the assays of any one of claims 8 to 16.

The array of the invention may be as above and additionally further comprising one or more oligonucleotides comprising any set of CpGs defined in the assays the invention, wherein the one or more oligonucleotides are hybridized to corresponding oligonucleotide probes of the array.

The invention also provides a hybridized array, wherein the array is obtainable by hybridizing to an array of the invention a group of oligonucleotides comprising any set of CpGs defined in the assays of the invention.

The invention also provides a process for making the hybridized array according to the invention, comprising contacting an array according to the invention with a group of oligonucleotides comprising any set of CpGs defined in the assays of the invention.

The invention also provides a method of treating breast cancer in an individual comprising:
a. predicting the presence or development of breast cancer in an individual by performing an assay of the invention;
b. stratifying the individual according to their risk of harbouring breast cancer or according to their risk of breast cancer development; and
c. administering one or more treatments to the individual based on their risk stratification.

The method of the invention may be performed above and additionally wherein the individual is stratified as having a low risk of harbouring breast cancer or a low risk of breast cancer development and the individual is subjected to treatment according to their risk, e.g. intensified screening.

The method of the invention may be performed above and additionally wherein the intensified screening comprises one or more mammography scans and/or breast MRI scans.

The method of the invention may be performed above and additionally wherein the individual is stratified as having a moderate risk of harbouring breast cancer or a moderate risk of breast cancer development and the individual is subjected to treatment according to their risk, e.g. intensified screening and/or administration of one or more doses of one or more of Mifeprestone, Aromatase inhibitors, Denosumab, “selective estrogen modulators” (SERMs) and “selective progesterone receptor modulators” (SPRMs).

The method of the invention may be performed above and additionally wherein the intensified screening comprises one or more mammography scans and/or breast MRI scans.

The method of the invention may be performed above and additionally wherein the individual is stratified as having a high risk of harbouring breast cancer or a high risk of breast cancer development and the individual is subjected to treatment according to their risk, e.g.:
a. intensified screening, optionally comprising one or mammography scans and/or breast MRI scans;
b. administration of one or more of Mifeprestone, Aromatase inhibitors, Denosumab, SERMs and SPRMs; and/or
c. bilateral mastectomy.

The invention also provides a method of monitoring the risk of an individual harbouring breast cancer or monitoring the risk of breast cancer development, the method comprising: (a) predicting the presence of breast cancer in an individual or predicting breast cancer development in an individual by performing an assay of the invention at a first time point; (b) predicting the presence of breast cancer in the individual or predicting breast cancer development in the individual by performing the assay of the invention at one or more further time points; and (c) monitoring any change in the individual's risk between time points.

The method of the invention may be performed above and additionally wherein the further time points are three monthly, six monthly, yearly or two yearly basis following an initial prediction.

The method of the invention may be performed above and additionally wherein the individual does not harbour breast cancer and harbours one or more genetic mutations that predispose the individual to an increased risk of developing breast cancer.

The method of the invention may be performed above and additionally wherein the individual harbours one or more mutations in a BRCA gene.

The method of the invention may be performed above and additionally wherein depending on the risk of the presence of breast cancer in the individual or risk of breast cancer development in the individual, one or more treatments are administered to the individual according to a method of the invention.

The method of the invention may be performed above and additionally wherein the individual has an increased risk of breast cancer development and wherein one or more treatments are administered to the individual according to a method of the invention a method of prophylaxis.

The method of the invention may be performed above and additionally wherein the one or more treatments administered to the individual comprises one or more doses of SPRMs.

The method of the invention may be performed above and additionally wherein the one or more doses of SPRMs comprises one or more doses of Mifepristone.

The method of the invention may be performed above and additionally wherein the method further comprises:
a. determining the proportion of epithelial cells in the sample from the individual between any two or more time points and assessing if the proportion changes between time points;
b. determining the proportion of fat cells in the sample from the individual between any two or more time points and assessing if the proportion changes between time points; and/or
c. determining differentiation characteristics of non-fat cells in the sample from the individual between any two or more time points and assessing if the proportion changes between time points.

The method of the invention may be performed above and additionally wherein:
a. an increase in the breast cancer index value and an increase in the proportion of epithelial cells; and/or
b. an increase in the breast cancer index value and a decrease in the proportion of fat cells; and/or
c. an increase in the breast cancer index value and an increase in differentiation of non-fat cells towards fat cells,
   indicates a negative response to the one or more treatments.

The method of the invention may be performed above and additionally wherein changes are made to the one or more treatments if a negative response is identified.

The method of the invention may be performed above and additionally wherein:
a. a decrease in the breast cancer index value and a decrease in the proportion of epithelial cells;
b. a decrease in the breast cancer index value and an increase in the proportion of fat cells; and/or
c. a decrease in the breast cancer index value and a decrease in differentiation of non-fat cells towards fat cells,
   indicates a positive response to the one or more treatments.

The method of the invention may be performed above and additionally wherein changes are made to the one or more treatments if a positive response is identified.

### BRIEF DESCRIPTION OF THE FIGURES

FIG. 1 shows an identification of differential methylation in cervical smear samples from breast cancer cases and controls.

A) Distribution of immune cell fraction in the Discovery Set. B) Distribution of eight non-epithelial cell subtypes inferred using HepiDISH (* p<0.05 Wilcoxon rank sum test). C) Example of cell-type specific DNAme. D) Distribution of estimated cell-type specific delta-beta values. E) Top ten tissue-specific patterns enriched in hyper-methylated CpGs. F) Top ten tissue-specific patterns enriched in hypo-methylated CpGs.

FIG. 2 shows discriminatory performance of the WID-BC-index in cervical smear samples.

A) Performance (as quantified by the AUC) as a function of the number of input CpGs to ridge and lasso classifiers. B) The WID-BC-index discriminates breast cancer cases from controls independently of immune contamination in the internal validation set. C) Receiver Operator Characteristic (ROC) curve corresponding to the optimal classifier in the internal validation set. D) The discriminatory performance of the WID-BC-index in an independent external validation set. E) ROC curve in the external validation set. F) Performance of sub-classifiers trained on different subsets of the 40,753 CpGs used to define the WID-BC-index. CpGs have been ranked according to the absolute values of the regression coefficients along the x-axis. The retained line refers to classifiers trained on only the top n CpGs. The removed group refers to classifiers trained after removing the top n CpGs. The binned line refers to classifiers trained on bins of 500 CpGs.

FIG. 3 shows association between WID-BC-index and epidemiological and clinical factors.

A) Age at time of consent. B) Number of relatives with breast cancer. C) Correlation with polygenic risk score (PRS) based on 303 SNPs on 345 women in the internal validation set. D) Tumour stage. E) Nodal status. F) Hormone receptor status (positive status defined as ER positive or PR positive). G) HER2 status. H) Grade. I) Histology. Panels A) and C) are based on the internal validation set, otherwise the entire discovery set was used. (* p<0.05 Wilcoxon rank sum test).

FIG. 4 shows performance of the WID-BC-index in matched buccal samples.

A) The WID-BC-index evaluated in buccal samples (corresponding to the internal validation set) discriminates between cases and controls. B) The corresponding ROC curve. C) Correlation between the WID-BC-index evaluated in matched cervical and buccal samples in the internal validation set.

FIG. 5 shows association of the WID-BC-index with fat cell content.

A) The WID-BC-index evaluated in ENCODE primary cells (pc) and in vitro differentiated cells (ivdc). B) The WID-index evaluated in ENCODE tissue samples.

FIG. 6 shows the WID-BC-index evaluated in breast tissue samples.

A) The WID-BC-index increases with the proportion of fat cells per sample. B) WID-BC-index after linear adjustment for fat content in breast tissue samples from healthy women, women with BRCA mutation, triple negative breast cancers (TNBC) and matched adjacent normal tissue. C) The proportion of epithelial cells in samples from the mifepristone trial before and after treatment with either mifepristone or placebo in women with BRCA mutation or healthy controls. D) WID-BC-index in samples from the mifepristone trial after linear adjustment for fat content before and after treatment with either mifepristone or placebo in women with BRCA mutation and healthy controls. (* p-value <0.05)

FIG. 7 shows an experimental design that led to the derivation of the WID-BC-index and the design of the evaluation of the WID-BC-index.

A) Surrogate samples (cervical and buccal swabs). B) ENCODE samples. C) Breast tissue samples.

FIG. 8 shows the distribution of immune cell subtypes in the external validation dataset.

A) Distribution of immune cell fraction in cancer cases and controls. B) Distribution of call subtypes inferred using HEpiDISH in the external validation dataset.

FIG. 9 shows the performance of alternative linear classifier.

A) Performance of ridge and lasso classifiers as a function of the number of input CpGs. The classifiers are based on a linear combination of inputs and do not contain any non-linear interaction terms (products of beta-value and IC-fraction). B) The optimal linear index (based on a ridge classifier with 10,000 inputs) performs well in low-IC samples, but the discriminatory signal diminishes for highly contaminated samples

FIG. 10 shows association between WID-BC-index and technical parameters.

A, B, C) The WID-BC-index discriminated between cases and controls when restricted to study centres that contributed predominantly cases or controls (based on internal and external validation data). D) Distribution of the WID-BC-index in controls that volunteered from the general population and women that presented at hospitals for benign women-specific conditions (Discovery set). E) The WID-BC-index is independent of the time between sample collection and processing (Discovery set).

FIG. 11 shows association between the WID-BC-index and additional epidemiological factors in the internal validation set.

A) Age at first live birth. B) Ethnicity. C) Hormone replacement therapy ever (post-menopausal only). D) Age of last period. E) Age at menarche. F) Menopausal status. G) Oral contraceptive use ever (pre-menopausal only). H) Parity (post-menopausal only).

FIG. 12 shows association between the WID-BC-index and epidemiological and clinical factors in the external validation dataset.

A) Age at time of consent. B) Number of relatives with breast cancer. C) Histology. D) Tumour stage. E) Nodal status. F) Hormone receptor status (positive status defined as ER positive or PR positive). G) HER2 status. H) Grade. I) Age at first live birth. J) Ethnicity. K) Hormone replacement therapy. L) Age of last period. M) Age at menarche. N) Menopausal status. 0) Oral contraceptive use. P) Parity.

FIG. 13 shows analysis of buccal samples.

A) Distribution of immune cell fraction in the buccal dataset. B) Distribution of immune cell subtypes in the buccal dataset. C) Performance of ridge and lasso classifiers (with non-linear interaction terms) trained on 269 buccal samples from the internal training set and validated on the internal validation set. D) Performance of classifiers trained and validated on the corresponding matched cervical samples.

FIG. 14 shows analysis of breast tissue samples.

A) Distribution of cell types in breast tissue samples from healthy women, women with BRCA mutation, and women with triple negative breast cancers (TNBC) and matched adjacent healthy tissue. B) Distribution of cell types in breast tissue samples from the mifepristone trial. C) Distribution of epithelial cells in samples with BRCA mutation from a combined with samples with BRCA mutation before treatment in B and compared to healthy controls from A and B. D) Distribution of epithelial cell fraction in the mifepristone trial groups using both matched and unmatched samples. E) Distribution of WID-BC-index in the mifepristone trial groups using both matched and unmatched samples. (* p-value <0.05, Wilcoxon rank sum test).

FIG. 15 shows a summary of epidemiological and clinical characteristics (cervical smear data).

A) Summary of epidemiological characteristics. B) Summary of clinical characteristics.

FIG. 16 shows GSEA top enriched pathways.

A) The top twenty enriched gene pathways based on hyper-methylated CpGs located in TSS200 regions. B) The top twenty pathways based on hypo-methylated CpGs. Pathways have been ranked by the normalised enrichment scores (NES). P-values have not been adjusted for multiplicity.

FIG. 17 shows a summary of WID-BC-index.

Summary of WID-BC-index. Odds ratios corresponding to each quartile of the WID-BC-index. Quartiles were defined by the controls of the internal validation set. Adjustment was based on a logistic regression model with age, menopausal status, age at menarche, number of first degree relatives with breast cancer, and BMI included as covariates.

FIG. 18 shows WID-BC-index thresholds applied to a population.

A) Receiver Operator Characteristic (ROC) curve corresponding to the optimal classifier in the internal validation set, wherein WID-BC-index thresholds capture either 50% of the population in which 94% of all breast cancers arise, 20% of the population in which 78% of all breast cancers arise or 3% of the population in which 34% of all breast cancers arise. B) Association between WID-BC-index with a horizontal lines indicating particular thresholds that mirror those described in the ROC curve of A.

### DETAILED DESCRIPTION OF THE INVENTION

The present invention is concerned with assays for predicting the development of breast cancer in an individual, by determining the methylation status of certain CpGs in DNA from the individual, deriving an index value based on the methylation status of the certain CpGs, and predicting the development of breast cancer in the individual based on the breast cancer index value.

“Predicting” in the context of the present invention relates to identifying a possible breast cancer index value that may be an indication of the presence of breast cancer in an individual or a particular risk of breast cancer development.

“Developing” in the context of the present invention may relate to cancer presently harboured by an individual that—on the basis of the individual's breast cancer index value—may further grow or metastasise within said individual. “Developing” in the context of the present invention may also relate to the absence of cancer in an individual however—on the basis of the individual's breast cancer index value—cancer may be predicted to manifest itself at some point in the future within said individual. Equally, “development” in the context of the present invention may relate to cancer presently harboured by an individual that—on the basis of the individual's breast cancer index value—may further grow or metastasise within said individual. “Development” in the context of the present invention may also relate to the absence of cancer in an individual however—on the basis of the individual's breast cancer index value—cancer may be predicted to manifest itself at some point in the future within said individual.

The present invention encompasses assays for predicting the presence or development of breast cancer in an individual, the assay comprising:
j. providing a sample which has been taken from the individual;
k. determining in DNA in the sample the methylation status of each CpG in a set of test CpGs selected from the panel of CpGs identified at nucleotide positions 61 to 62 in SEQ ID NOs 1 to 40,753;
l. deriving a breast cancer index value based on the methylation status of the test CpGs; and
m. predicting the presence or development of breast cancer in the individual based on the breast cancer index value;

wherein the assay is characterised as having an area under the curve (AUC) of 0.6 or more as determined by receiver operating characteristics (ROC).

Assays according to the present invention provide a statistically robust pool of CpGs whose methylation status can be determined to provide a reliable prediction of the presence or development of breast cancer in an individual. As exemplified by the data described herein, the pool of CpGs identified by the inventors can be used in an assay of the invention having an AUC of 0.6 or more. Furthermore, subsets of the provided pool of CpGs can be assayed according to the present invention thus enabling stratification of individuals according to their risk of harbouring breast cancer or of breast cancer development with statistically robust sensitivity and specificity, as determined by receiver operating characteristics.

### Identification of CpGs and Assessment Methylation Status

Methylation of DNA is a recognised form of epigenetic modification which has the capability of altering the expression of genes and other elements such as microRNAs [[1]]. In cancer development and progression, methylation may have the effect of e.g. silencing tumor suppressor genes and/or increasing the expression of oncogenes. Other forms of dysregulation may occur as a result of methylation. Methylation of DNA occurs at discrete loci which are predominately dinucleotides consisting of a CpG motif, but may also occur at CHH motifs (where H is A, C, or T). During methylation, a methyl group is added to the fifth carbon of cytosine bases to create methylcytosine.

Methylation can occur throughout the genome and is not limited to regions with respect to an expressed sequence such as a gene. Methylation typically, but not always, occurs in a promoter or other regulatory region of an expressed sequence such as enhancer elements. Most typically, the methylation status of CpGs is clustered in CpG islands, for example CpG islands present in the regulatory regions of genes, especially in their promoter regions.

Typically, an assessment of DNA methylation status involves analysing the presence or absence of methyl groups in DNA, for example methyl groups on the 5 position of one or more cytosine nucleotides. Preferably, the methylation status of one or more cytosine nucleotides present as a CpG dinucleotide (where C stands for Cytosine, G for Guanine and p for the phosphate group linking the two) is assessed.

A variety of techniques are available for the identification and assessment of CpG methylation status, as will be outlined briefly below. The assays described herein encompass any suitable technique for the determination of CpG methylation status.

Methyl groups are lost from a starting DNA molecule during conventional in vitro handling steps such as PCR. To avoid this, techniques for the detection of methyl groups commonly involve the preliminary treatment of DNA prior to subsequent processing, in a way that preserves the methylation status information of the original DNA molecule. Such preliminary techniques involve three main categories of processing, i.e. bisulphite modification, restriction enzyme digestion and affinity-based analysis. Products of these techniques can then be coupled with sequencing or array-based platforms for subsequent identification or qualitative assessment of CpG methylation status.

Techniques involving bisulphite modification of DNA have become the most common assays for detection and assessment of methylation status of CpG dinucleotides. Treatment of DNA with bisulphite, e.g. sodium bisulphite, converts cytosine bases to uracil bases, but has no effect on 5-methylcytosines. Thus, the presence of a cytosine in bisulphite-treated DNA is indicative of the presence of a cytosine base which was previously methylated in the starting DNA molecule. Such cytosine bases can be detected by a variety of techniques. For example, primers specific for unmethylated versus methylated DNA can be generated and used for PCR-based identification of methylated CpG dinucleotides. DNA may be amplified, either before or after bisulphite conversion. A separation/capture step may be performed, e.g. using binding molecules such as complementary oligonucleotide sequences. Standard and next-generation DNA sequencing protocols can also be used.

In other approaches, methylation-sensitive enzymes can be employed which digest or cut only in the presence of methylated DNA. Analysis of resulting fragments is commonly carried out using mircroarrays.

Affinity-based techniques exploit binding interactions to capture fragments of methylated DNA for the purposes of enrichment. Binding molecules such as anti-5-methylcytosine antibodies are commonly employed prior to subsequent processing steps such as PCR and sequencing.

Olkhov-Mitsel and Bapat (2012) [[1]] provide a comprehensive review of techniques available for the identification and assessment of biomarkers involving methylcytosine.

For the purposes of assessing the methylation status of the CpG-based biomarkers characterised and described herein, any suitable assay can be employed.

Assays described herein may comprise determining methylation status of CpGs by bisulphite converting the DNA. Preferred assays involve bisulphite treatment of DNA, including amplification of the identified CpG loci for methylation specific PCR and/or sequencing and/or assessment of the methylation status of target loci using methylation-discriminatory microarrays.

Amplification of CpG loci can be achieved by a variety of approaches. Preferably, CpG loci are amplified using PCR. A variety of PCR-based approaches may be used. For example, methylation-specific primers may be hybridized to DNA containing the CpG sequence of interest. Such primers may be designed to anneal to a sequence derived from either a methylated or non-methylated CpG locus. Following annealing, a PCR reaction is performed and the presence of a subsequent PCR product indicates the presence of an annealed CpG of identifiable sequence. In such assays, DNA is bisulphite converted prior to amplification. Such techniques are commonly referred to as methylation specific PCR (MSP) [[2]].

In other techniques, PCR primers may anneal to the CpG sequence of interest independently of the methylation status, and further processing steps may be used to determine the status of the CpG. Assays are designed so that the CpG site(s) are located between primer annealing sites. This assay scheme is used in techniques such as bisulphite genomic sequencing [[3]], COBRA [[4]], Ms-SNuPE [[5]]. In such assay, DNA can be bisulphite converted before or after amplification.

Small-scale PCR approaches may be used. Such approaches commonly involve mass partitioning of samples (e.g. digital PCR). These techniques offer robust accuracy and sensitivity in the context of a highly miniaturised system (pico-liter sized droplets), ideal for the subsequent handling of small quantities of DNA obtainable from the potentially small volume of cellular material present in biological samples, particularly urine samples. A variety of such small-scale PCR techniques are widely available. For example, microdroplet-based PCR instruments are available from a variety of suppliers, including RainDance Technologies, Inc. (Billerica, Mass.; http://raindancetech.com/) and Bio-Rad, Inc. (http://www.bio-rad.com/). Microarray platforms may also be used to carry out small-scale PCR. Such platforms may include microfluidic network-based arrays e.g. available from Fluidigm Corp. (www.fluidigm.com).

Following amplification of CpG loci, amplified PCR products may be coupled to subsequent analytical platforms in order to determine the methylation status of the CpGs of interest. For example, the PCR products may be directly sequenced to determine the presence or absence of a methylcytosine at the target CpG or analysed by array-based techniques.

Any suitable sequencing techniques may be employed to determine the sequence of target DNA. In the assays of the present invention the use of high-throughput, so-called “second generation”, “third generation” and “next generation” techniques to sequence bisulphite-treated DNA can be used.

In second generation techniques, large numbers of DNA molecules are sequenced in parallel. Typically, tens of thousands of molecules are anchored to a given location at high density and sequences are determined in a process dependent upon DNA synthesis. Reactions generally consist of successive reagent delivery and washing steps, e.g. to allow the incorporation of reversible labelled terminator bases, and scanning steps to determine the order of base incorporation. Array-based systems of this type are available commercially e.g. from Illumina, Inc. (San Diego, Calif.; http://www.illumina.com/).

Third generation techniques are typically defined by the absence of a requirement to halt the sequencing process between detection steps and can therefore be viewed as real-time systems. For example, the base-specific release of hydrogen ions, which occurs during the incorporation process, can be detected in the context of microwell systems (e.g. see the Ion Torrent system available from Life Technologies; http://www.lifetechnologies.com/). Similarly, in pyrosequencing the base-specific release of pyrophosphate (PPi) is detected and analysed. In nanopore technologies, DNA molecules are passed through or positioned next to nanopores, and the identities of individual bases are determined following movement of the DNA molecule relative to the nanopore. Systems of this type are available commercially e.g. from Oxford Nanopore (https://www.nanoporetech.com/). In an alternative assay, a DNA polymerase enzyme is confined in a “zero-mode waveguide” and the identity of incorporated bases are determined with florescence detection of gamma-labeled phosphonucleotides (see e.g. Pacific Biosciences; http://www.pacificbiosciences.com/).

In other assays in accordance with the invention sequencing steps may be omitted. For example, amplified PCR products may be applied directly to hybridization arrays based on the principle of the annealing of two complementary nucleic acid strands to form a double-stranded molecule. Hybridization arrays may be designed to include probes which are able to hybridize to amplification products of a CpG and allow discrimination between methylated and non-methylated loci. For example, probes may be designed which are able to selectively hybridize to an CpG locus containing thymine, indicating the generation of uracil following bisulphite conversion of an unmethylated cytosine in the starting template DNA. Conversely, probes may be designed which are able to selectively hybridize to a CpG locus containing cytosine, indicating the absence of uracil conversion following bisulphite treatment. This corresponds with a methylated CpG locus in the starting template DNA.

Following the application of a suitable detection system to the array, computer-based analytical techniques can be used to determine the methylation status of a CpG. Detection systems may include, e.g. the addition of fluorescent molecules following a methylation status-specific probe extension reaction. Such techniques allow CpG status determination without the specific need for the sequencing of CpG amplification products. Such array-based discriminatory probes may be termed methylation-specific probes.

Any suitable methylation-discriminatory microarrays may be employed to assess the methylation status of the CpGs described herein. A preferred methylation-discriminatory microarray system is provided by Illumina, Inc. (San Diego, Calif.; http://www.illumina.com/). In particular, the Infinium MethylationEPIC BeadChip array and the Infinium HumanMethylation450 BeadChip array systems may be used to assess the methylation status of CpGs for predicting cancer development as described herein. Such a system exploits the chemical modifications made to DNA following bisulphite treatment of the starting DNA molecule. Briefly, the array comprises beads to which are coupled oligonucleotide probes specific for DNA sequences corresponding to the unmethylated form of a CpG, as well as separate beads to which are coupled oligonucleotide probes specific for DNA sequences corresponding to the methylated form of an CpG. Candidate DNA molecules are applied to the array and selectively hybridize, under appropriate conditions, to the oligonucleotide probe corresponding to the relevant epigenetic form. Thus, a DNA molecule derived from a CpG which was methylated in the corresponding genomic DNA will selectively attach to the bead comprising the methylation-specific oligonucleotide probe, but will fail to attach to the bead comprising the non-methylation-specific oligonucleotide probe. Single-base extension of only the hybridized probes incorporates a labeled ddNTP, which is subsequently stained with a fluorescence reagent and imaged. The methylation status of the CpG is determined by calculating the ratio of the fluorescent signal derived from the methylated and unmethylated sites.

Because the cancer-specific diagnostic CpG biomarkers defined herein were initially identified using the Illumina® Infinium MethylationEPIC Beadchip array and Infinium HumanMethylation450 BeadChip array systems, the same chip systems can be used to interrogate those same CpGs in the assays described herein. Alternative or customised arrays could, however, be employed to interrogate the cancer-specific CpG biomarkers defined herein, provided that they comprise means for interrogating all CpG for a given assay, as defined herein.

Techniques involving combinations of the above-described assays may also be used. For example, DNA containing CpG sequences of interest may be hybridized to microarrays and then subjected to DNA sequencing to determine the status of the CpG as described above.

In the assays described above, sequences corresponding to CpG loci may also be subjected to an enrichment process if desired. DNA containing CpG sequences of interest may be captured by binding molecules such as oligonucleotide probes complementary to the CpG target sequence of interest. Sequences corresponding to CpG loci may be captured before or after bisulphite conversion or before or after amplification. Probes may be designed to be complementary to bisulphite converted DNA. Captured DNA may then be subjected to further processing steps to determine the status of the CpG, such as DNA sequencing steps.

Capture/separation steps may be custom designed. Alternatively a variety of such techniques are available commercially, e.g. the SureSelect target enrichment system available from Agilent Technologies (http://www.agilent.com/home). In this system biotinylated “bait” or “probe” sequences (e.g. RNA) complementary to the DNA containing CpG sequences of interest are hybridized to sample nucleic acids. Streptavidin-coated magnetic beads are then used to capture sequences of interest hybridized to bait sequences. Unbound fractions are discarded. Bait sequences are then removed (e.g. by digestion of RNA) thus providing an enriched pool of CpG target sequences separated from non-CpG sequences. Template DNA may be subjected to bisulphite conversion and target loci amplified by small-scale PCR such as microdroplet PCR using primers which are independent of the methylation status of the CpG. Following amplification, samples may be subjected to a capture step to enrich for PCR products containing the target CpG, e.g. captured and purified using magnetic beads, as described above. Following capture, a standard PCR reaction is carried out to incorporate DNA sequencing barcodes into CpG-containing amplicons. PCR products are again purified and then subjected to DNA sequencing and analysis to determine the presence or absence of a methylcytosine at the target genomic CpG [[6]].

The CpG biomarker loci defined herein are identified e.g. by Illumina® identifiers (IlmnID). These CpG loci identifiers refer to individual CpG sites used in the commercially available Illumina® Infinium Methylation EPIC BeadChip kit and Illumina® Infinium Human Methylation450 BeadChip kit. The identity of each CpG site represented by each CpG loci identifier is publicly available from the Illumina, Inc. website under reference to the CpG sites used in the Infinium Methylation EPIC BeadChip kit and the Infinium Human Methylation450 BeadChip kit.

To complement evolving public databases to provide accurate CpG loci identifiers and strand orientation, Illumina® has developed a method to consistently designate CpG loci based on the actual or contextual sequence of each individual CpG locus. To unambiguously refer to CpG loci in any species, Illumina® has developed a consistent and deterministic CpG loci database to ensure uniformity in the reporting of methylation data. The Illumina® method takes advantage of sequences flanking a CpG locus to generate a unique CpG locus cluster ID. This number is based on sequence information only and is unaffected by genome version. Illumina's standardized nomenclature also parallels the TOP/BOT strand nomenclature (which indicates the strand orientation) commonly used for single nucleotide polymorphism (SNP) designation.

Illumina® Identifiers for the Infinium MethylationEPIC BeadChip and Infinium Human Methylation450 BeadChip system are also available from public repositories such as Gene Expression Omnibus (GEO) (http://www.ncbi.nlm.nih.gov/geo/).

By assessing the methylation status of a CpG it is meant that a determination is made as to whether a given CpG is methylated or unmethylated. In addition, it is meant that a determination is made as to the degree to which a given CpG site is methylated across a population of CpG loci in a sample.

In a preferred assay of the invention, CpG methylation status is measured indirectly using a detection system such as fluorescence. In a preferred system, a methylation-discriminatory microarray is used. In a preferred method of calculating the degree of methylation of a given CpG, the Illumina® definition of beta-values is used (see Examples for further details). The Illumina® methylation beta-value of a specific CpG site is calculated from the intensity of the methylated (M) and unmethylated (U) alleles, as the ratio of fluorescent signals β=Max(M,0)/[Max(M,0)+Max(U,0)+100]. On this scale, 0<β<1, β values close to 1 (0) indicate 100% methylation (no methylation).

The inventors initially sought to examine epigenome-wide DNAme analysis in breast tissue samples and in samples derived from anatomical sites other than the breast who had been diagnosed with breast cancer and in matched controls. This led to the surprising establishment of a WID-BC-index (Women's risk Identification for Breast Cancer index) based on DNAme signatures that are capable of identifying women with breast cancer (see Examples for further details). Surprisingly, the signatures were shown to be changeable in response to breast cancer therapies, thus indicating the dynamic nature of the identified predictive DNAme signature, and thus surprisingly indicating that the DNAme signatures of the invention can be used to monitor breast cancer.

A CpG as defined herein refers to the CG dinucleotide motif identified in relation to each SEQ ID NO. and Illumina Identifier (Ilmn ID), and chromosome position of the sequence as listed in the sequence listing, wherein the cytosine base of the dinucleotide may (or may not) be modified. Thus by determining the methylation status of a CpG defined by or identified in a given SEQ ID NO., it is meant that a determination is made as to the methylation status of the cytosine of the CG dinucleotide motif identified in square brackets in each sequence shown in sequence listing, accepting that variation in the sequence upstream and downstream of any given CpG may exist due to sequencing errors or variation between individuals.

### Cancer-Related CpG Groups for Analysis

In any of the assays described herein, in DNA derived from cells in the sample the methylation status of each CpG in a set of test CpGs selected from a panel of CpGs may be determined.

The assays may involve determining the methylation status of each CpG in a set of test CGs selected from the panel of CpGs identified at nucleotide positions 61 to 62 in SEQ ID NOs 1 to 40,753.

The assays may involve determining the methylation status of each CpG in a set of test CGs selected from the panel of CpGs identified at nucleotide positions 61 to 62 in SEQ ID NOs 1 to 40,753, wherein the set of CpGs comprises at least one of the CpGs identified in SEQ ID NOs 1 to 40,753.

The assays may involve determining the methylation status of each CpG in a set of test CGs selected from the panel of CpGs identified at nucleotide positions 61 to 62 in SEQ ID NOs 1 to 40,753, wherein the set of CpGs comprises any number of the CpGs identified in SEQ ID NOs 1 to 40,753.

The assays may involve determining the methylation status of each CpG in a set of test CGs selected from the panel of CpGs identified at nucleotide positions 61 to 62 in SEQ ID NOs 1 to 40,753, wherein the set of CpGs comprises between 1 and 500 of the CpGs identified in SEQ ID NOs 1 to 40,753.

The assays may involve determining the methylation status of each CpG in a set of test CGs selected from the panel of CpGs identified at nucleotide positions 61 to 62 in SEQ ID NOs 1 to 40,753, wherein the set of CpGs comprises at least 500 CpGs selected from the CpGs identified in SEQ ID NOs 1 to 40,753.

The assays may involve determining the methylation status of each CpG in a set of test CGs selected from the panel of CpGs identified at nucleotide positions 61 to 62 in SEQ ID NOs 1 to 40,753, wherein the set of CpGs comprises at least 500 CpGs selected from the CpGs identified in SEQ ID NOs 1 to 40,753 and wherein the set of at least 500 CpGs comprises at least the CpGs identified in SEQ ID NOs 1 to 500 and identified at nucleotide positions 61 to 62.

The assays may involve determining the methylation status of each CpG in a set of test CGs selected from the panel of CpGs identified at nucleotide positions 61 to 62 in SEQ ID NOs 1 to 40,753, wherein the set of CpGs comprises at least 1000 CpGs selected from the CpGs identified in SEQ ID NOs 1 to 40,753.

The assays may involve determining the methylation status of each CpG in a set of test CGs selected from the panel of CpGs identified at nucleotide positions 61 to 62 in SEQ ID NOs 1 to 40,753, wherein the set of CpGs comprises at least 1000 CpGs selected from the CpGs identified in SEQ ID NOs 1 to 40,753 and wherein the set of at least 1000 CpGs comprises at least the CpGs identified in SEQ ID NOs 1 to 1000 and identified at nucleotide positions 61 to 62.

The assays may involve determining the methylation status of each CpG in a set of test CGs selected from the panel of CpGs identified at nucleotide positions 61 to 62 in SEQ ID NOs 1 to 40,753, wherein the set of CpGs comprises at least 2000 CpGs selected from the CpGs identified in SEQ ID NOs 1 to 40,753.

The assays may involve determining the methylation status of each CpG in a set of test CGs selected from the panel of CpGs identified at nucleotide positions 61 to 62 in SEQ ID NOs 1 to 40,753, wherein the set of CpGs comprises at least 2000 CpGs selected from the CpGs identified in SEQ ID NOs 1 to 40,753 and wherein the set of at least 2000 CpGs comprises at least the CpGs identified in SEQ ID NOs 1 to 2000 and identified at nucleotide positions 61 to 62.

The assays may involve determining the methylation status of each CpG in a set of test CGs selected from the panel of CpGs identified at nucleotide positions 61 to 62 in SEQ ID NOs 1 to 40,753, wherein the set of CpGs comprises at least 10,000 CpGs selected from the CpGs identified in SEQ ID NOs 1 to 40,753.

The assays may involve determining the methylation status of each CpG in a set of test CGs selected from the panel of CpGs identified at nucleotide positions 61 to 62 in SEQ ID NOs 1 to 40,753, wherein the set of CpGs comprises at least 10,000 CpGs selected from the CpGs identified in SEQ ID NOs 1 to 40,753 and wherein the set of at least 10,000 CpGs comprises at least the CpGs identified in SEQ ID NOs 1 to 10,000 and identified at nucleotide positions 61 to 62.

The assays may involve determining the methylation status of each CpG in a set of test CGs selected from the panel of CpGs identified at nucleotide positions 61 to 62 in SEQ ID NOs 1 to 40,753, wherein the set of CpGs comprises at least 40,753 CpGs selected from the CpGs identified at nucleotide positions 61 to 62 in SEQ ID NOs 1 to 40,753.

### Breast Cancer Index

In view observations described herein (see Examples), the inventors derived an index based on an analysis of the CpG beta value (as defined above) for use assays for predicting the presence or development of breast cancer in an individual. Any of the assays described herein may involve deriving a breast cancer index value based on the methylation of status of the test CpGs in a sample provided from an individual. Any of the methods described herein may involve predicting the presence or development of breast cancer in an individual.

The breast cancer index value may be derived by any suitable means. Preferably, the breast cancer index value may be derived by assessing the methylation status of the test CpGs in a sample provided from an individual. The methylation status of the CpGs may be determined by any suitable means. Preferably, the step of determining the methylation status of each CpG in the set of test CpGs comprises determining methylation beta-values for each one of the test CpGs. Deriving the breast cancer index value may involve providing a methylation beta-value data set comprising methylation beta-values for each test CpG. Deriving the breast cancer index value may also involve estimating the fraction of contaminating DNA within the DNA provided from a sample. Contaminating DNA may be DNA originating from a particular source organism, tissue or cell type. Preferably the contaminating DNA originates from one or more different cell types to one or more cell types of interest. A cell type of interest may particularly be an epithelial cell or hormone sensing cell. Estimating the fraction of contaminating DNA can be performed by any suitable means and at any suitable stage in the assays described herein after the step of providing a sample which has been take from an individual. The assays described herein involve estimating a contaminating DNA fraction within DNA in the sample by any suitable means. Preferably, the contaminating DNA fraction for the sample is estimated via any suitable bioinformatics analysis tool. A bioinformatics analysis tool that may be used to estimate a contaminating DNA fraction may be EpIDISH.

The contaminating DNA is preferably from immune cells. Preferably, in any of the assays described herein, the step of deriving the breast cancer index value based on the methylation status of the test CpGs comprises providing a methylation beta-value data set comprising the methylation beta-values for each test CpG and estimating an immune cell DNA fraction within the DNA provided from the sample. The estimated immune cell DNA fraction may be controlled for in any algorithm applied to the methylation beta-value data set to obtain a breast cancer index value in accordance with the present invention.

It is desirable to estimate the fraction of contaminating DNA from the one or more cell types that are different to the one or more cell types of interest because the breast cancer index value used for predicting the presence or development of breast cancer in an individual may, in some instances, only be reliably derived from determining the methylation status of a set of CpGs from DNA of a particular cell type of interest. Particularly, methylation status beta-values may differ in the one or more cell types of interest within a sample relative to methylation status beta-values in contaminating DNA from different cell types within the same sample. Thus, the derived breast cancer index value may have a decreased predictive power without estimating and controlling for the contaminating DNA fraction within the DNA provided from the sample. Preferably the assay involves estimating an immune cell DNA fraction within the DNA provided from the sample.

Any of the assays described herein comprising a step of deriving a breast cancer index value based on the methylation status of the test CpGs may further comprise applying an algorithm to the methylation beta-value dataset to obtain the breast cancer index value. Preferably, in any of the assays described herein, the step of deriving the breast cancer index value based on the methylation status of the test CpGs comprises providing a methylation beta-value data set comprising the methylation beta-values for each test CpG, estimating an immune cell DNA fraction within the DNA provided from the sample and applying an algorithm to the methylation beta-value data set to obtain the breast cancer index value.

In any of the assays described herein, the breast cancer index value may be calculated by any suitable formula. Preferably, the breast cancer index value is termed Women's risk Identification for Breast Cancer Index (WID-BC-index) and is calculated by an algorithm according to the following formula:

$$WID - BC - index = {\sum }_{i = 1}^{n} \frac{μ - ( {a}_{i} + {b}_{i} ρ ) {β}_{i}}{σ}$$

where n refers to the number of CpGs in the set of test CpGs, ρ∈[0,1] is the immune cell DNA fraction for the sample, β₁, . . . , β₁, are methylation beta-values (between 0 and 1), a₁, . . . , aₙ and b₁, . . . , bₙ are real valued coefficients and μ and σ are real valued parameters used to scale the index.

In any of the assays described herein, the WID-BC-index algorithm applies real value coefficients (a₁, . . . , aₙ and b₁, . . . , bₙ) inferred by initially training a dataset (this dataset in the exemplary embodiments of the invention described in the examples consisted of 190 breast cancer cases and 508 controls) to fit a ridge classifier using the R package glmnet with a mixing parameter value of alpha=0 (ridge penalty) and binomial response type. Ten-fold cross-validation was used internally by the cv.glmnet function in order to determine the optimal value of the regularisation parameter lambda. For individual v denote beta values from the n CpGs as β₁^{v}, . . . , βₙ^{v} and denote the immune cell fraction as ρⁿ. The following terms were used as inputs to the ridge classifier
β₁^{v}, ρ^{v}β₁^{v}, . . . , βₙ^{v}, ρ^{v}βₙ^{v}
   The coefficients a₁, . . . , aₙ and b₁, . . . , bₙ are obtained from the fitted ridge model. The coefficients a₁, . . . , aₙ correspond to the terms β₁^{v}, . . . , βₙ^{v} and the coefficients b₁, . . . , bₙ correspond to the terms ρ^{v}β₁^{v}, . . . , ρ^{v}βₙ^{v}. Thus, any suitable a₁, . . . , aₙ and b₁, . . . , bₙ real valued coefficients may be applied to the WID-BC-index in any of the assays described herein. The following quantity was computed for each individual v in the training set:

$${x}_{υ} = {\sum }_{i = 1}^{n} ( {a}_{i} + {b}_{i} {ρ}^{υ} ) {β}_{i}^{υ}$$

The value of the parameters μ and σ are given by the mean and standard deviation of xᵥ in the training dataset respectively. Thus, any suitable μ and σ real valued parameters may be applied to the WID-BC-index in any of the assays described herein. Any suitable training data set may be applied to the assays described herein in order to infer real value parameters and coefficients that can subsequently be applied to the WID-BC-index formula according to the present invention. Exemplary ways of utilising a training dataset in accordance with the present invention are further described in the ‘Statistical Analyses for Classifier Development’ section of the Materials and Methods section of the Examples.

Exemplary μ and σ real valued parameters are provided in Table 1 for CpG subsets identified in SEQ ID NOs 1 to 40,753. These real valued parameters may be applied to any of the assays described herein wherein the real parameters correspond to any one of the sets of CpGs identified in SEQ ID NOs 1 to 40,753 and set out in the left hand column of Table 1.

### Bioinformatic Tools and Statistical Metrics for CpG-Based Assays

Software programs which aid in the in silico analysis of bisulphite converted DNA sequences and in primer design for the purposes of methylation-specific analyses are generally available and have been described previously [[7]] [[8]] [[9]].

In risk models for predicting cancer, a receiver-operating-characteristic (ROC) curve analysis is often used, in which the area under the curve (AUC) is assessed [[10]]. Each point on the ROC curve shows the effect of a rule for turning a risk/likelihood estimate into a prediction of the presence or development of cancer in an individual. The AUC measures how well the model discriminates between case subjects and control subjects. An ROC curve that corresponds to a random classification of case subjects and control subjects is a straight line with an AUC of 50%. An ROC curve that corresponds to perfect classification has an AUC of 100%.

In any of the methods described herein, the 95% confidence interval for the ROC AUC may be between 0.60 and 1.

In any of the methods described herein, the interval may be defined as a range having as an upper limit any number between 0.60 and 1. The upper limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99 or 1.00.

In any of the methods described herein, the interval may be defined as a range having as a lower limit any number between 0.60 and 1. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99 or 1.00.

In any of the methods described herein, the interval range may comprise any of the above lower limit numbers combined with any of the above upper limit numbers as appropriate.

Preferably, the upper limit number is 1. Thus, the 95% confidence ROC AUC interval may be defined as a range having an upper limit of 1 and as a lower limit any number between 0.60 and 1. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99 or 1.00.

The upper limit number may be 0.99. Thus, the 95% confidence ROC AUC interval may be defined as a range having an upper limit of 0.99 and as a lower limit any number between 0.60 and 0.99. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98 or 0.99.

The upper limit number may be 0.98. Thus, the 95% confidence ROC AUC interval may be defined as a range having an upper limit of 0.98 and as a lower limit any number between 0.60 and 0.98. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97 or 0.98.

The upper limit number may be 0.97. Thus, the 95% confidence ROC AUC interval may be defined as a range having an upper limit of 0.97 and as a lower limit any number between 0.60 and 0.97. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96 or 0.97.

The upper limit number may be 0.96. Thus, the 95% confidence ROC AUC interval may be defined as a range having an upper limit of 0.96 and as a lower limit any number between 0.60 and 0.96. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95 or 0.96.

The upper limit number may be 0.95. Thus, the 95% confidence ROC AUC interval may be defined as a range having an upper limit of 0.95 and as a lower limit any number between 0.60 and 0.95. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94 or 0.95.

The upper limit number may be 0.94. Thus, the 95% confidence ROC AUC interval may be defined as a range having an upper limit of 0.94 and as a lower limit any number between 0.60 and 0.94. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93 or 0.94.

The upper limit number may be 0.93. Thus, the 95% confidence ROC AUC interval may be defined as a range having an upper limit of 0.93 and as a lower limit any number between 0.60 and 0.93. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92 or 0.93.

The upper limit number may be 0.92. Thus, the 95% confidence ROC AUC interval may be defined as a range having an upper limit of 0.92 and as a lower limit any number between 0.60 and 0.92. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91 or 0.92.

The upper limit number may be 0.91. Thus, the 95% confidence ROC AUC interval may be defined as a range having an upper limit of 0.91 and as a lower limit any number between 0.60 and 0.91. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90 or 0.91.

The upper limit number may be 0.90. Thus, the 95% confidence ROC AUC interval may be defined as a range having an upper limit of 0.90 and as a lower limit any number between 0.60 and 0.90. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89 or 0.90.

The upper limit number may be 0.89. Thus, the 95% confidence ROC AUC interval may be defined as a range having an upper limit of 0.89 and as a lower limit any number between 0.60 and 0.89. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88 or 0.89.

The upper limit number may be 0.88. Thus, the 95% confidence ROC AUC interval may be defined as a range having an upper limit of 0.88 and as a lower limit any number between 0.60 and 0.88. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87 or 0.88.

The upper limit number may be 0.87. Thus, the 95% confidence ROC AUC interval may be defined as a range having an upper limit of 0.87 and as a lower limit any number between 0.60 and 0.87. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86 or 0.87.

The upper limit number may be 0.86. Thus, the 95% confidence ROC AUC interval may be defined as a range having an upper limit of 0.86 and as a lower limit any number between 0.60 and 0.86. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85 or 0.86.

The upper limit number may be 0.85. Thus, the 95% confidence ROC AUC interval may be defined as a range having an upper limit of 0.85 and as a lower limit any number between 0.60 and 0.85. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84 or 0.85.

The upper limit number may be 0.84. Thus, the 95% confidence ROC AUC interval may be defined as a range having an upper limit of 0.84 and as a lower limit any number between 0.60 and 0.84. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83 or 0.84.

The upper limit number may be 0.83. Thus, the 95% confidence ROC AUC interval may be defined as a range having an upper limit of 0.83 and as a lower limit any number between 0.60 and 0.83. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82 or 0.83.

The upper limit number may be 0.82. Thus, the 95% confidence ROC AUC interval may be defined as a range having an upper limit of 0.82 and as a lower limit any number between 0.60 and 0.82. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81 or 0.82.

The upper limit number may be 0.81. Thus, the 95% confidence ROC AUC interval may be defined as a range having an upper limit of 0.81 and as a lower limit any number between 0.60 and 0.81. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80 or 0.81.

The upper limit number may be 0.80. Thus, the 95% confidence ROC AUC interval may be defined as a range having an upper limit of 0.80 and as a lower limit any number between 0.60 and 0.80. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79 or 0.80.

The upper limit number may be 0.79. Thus, the 95% confidence ROC AUC interval may be defined as a range having an upper limit of 0.79 and as a lower limit any number between 0.60 and 0.79. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78 or 0.79.

The upper limit number may be 0.78. Thus, the 95% confidence ROC AUC interval may be defined as a range having an upper limit of 0.78 and as a lower limit any number between 0.60 and 0.78. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77 or 0.78.

The upper limit number may be 0.77. Thus, the 95% confidence ROC AUC interval may be defined as a range having an upper limit of 0.77 and as a lower limit any number between 0.60 and 0.77. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76 or 0.77.

The upper limit number may be 0.76. Thus, the 95% confidence ROC AUC interval may be defined as a range having an upper limit of 0.76 and as a lower limit any number between 0.60 and 0.76. The lower limit number may be 0.60, 0.61, 0.62, 0.63, 0.64, 0.65, 0.66, 0.67, 0.68, 0.69, 0.70, 0.71, 0.72, 0.73, 0.74, 0.75 or 0.76.

The upper limit number may be 0.75. Thus, the 95% confidence ROC AUC interval may be defined as a range having an upper limit of 0.75 and as a lower limit any number between 0.60 and 0.75. The lower limit number may be 0.60, 0.61, 0.62, 0.63, 0.64, 0.65, 0.66, 0.67, 0.68, 0.69, 0.70, 0.71, 0.72, 0.73, 0.74 or 0.75.

The upper limit number may be 0.74. Thus, the 95% confidence ROC AUC interval may be defined as a range having an upper limit of 0.74 and as a lower limit any number between 0.60 and 0.74. The lower limit number may be 0.60, 0.61, 0.62, 0.63, 0.64, 0.65, 0.66, 0.67, 0.68, 0.69, 0.70, 0.71, 0.72, 0.73 or 0.74.

The upper limit number may be 0.73. Thus, the 95% confidence ROC AUC interval may be defined as a range having an upper limit of 0.73 and as a lower limit any number between 0.60 and 0.73. The lower limit number may be 0.60, 0.61, 0.62, 0.63, 0.64, 0.65, 0.66, 0.67, 0.68, 0.69, 0.70, 0.71, 0.72 or 0.73.

The upper limit number may be 0.72. Thus, the 95% confidence ROC AUC interval may be defined as a range having an upper limit of 0.72 and as a lower limit any number between 0.60 and 0.72. The lower limit number may be 0.60, 0.61, 0.62, 0.63, 0.64, 0.65, 0.66, 0.67, 0.68, 0.69, 0.70, 0.71 or 0.72.

The upper limit number may be 0.71. Thus, the 95% confidence ROC AUC interval may be defined as a range having an upper limit of 0.71 and as a lower limit any number between 0.60 and 0.71. The lower limit number may be 0.60, 0.61, 0.62, 0.63, 0.64, 0.65, 0.66, 0.67, 0.68, 0.69, 0.70 or 0.71.

The upper limit number may be 0.70. Thus, the 95% confidence ROC AUC interval may be defined as a range having an upper limit of 0.70 and as a lower limit any number between 0.60 and 0.70. The lower limit number may be 0.60, 0.61, 0.62, 0.63, 0.64, 0.65, 0.66, 0.67, 0.68, 0.69 or 0.70.

The assays may involve determining the methylation status of each CpG in a set of test CGs selected from the panel of CpGs identified at nucleotide positions 61 to 62 in SEQ ID NOs 1 to 40,753, wherein the set of CpGs comprises at least 500 CpGs selected from the CpGs identified in SEQ ID NOs 1 to 40,753, preferably wherein the assay is characterised as having an AUC of at least 0.73.

The assays may involve determining the methylation status of each CpG in a set of test CGs selected from the panel of CpGs identified at nucleotide positions 61 to 62 in SEQ ID NOs 1 to 40,753, wherein the set of CpGs comprises at least 1000 CpGs selected from the CpGs identified in SEQ ID NOs 1 to 40,753, preferably wherein the assay is characterised as having an AUC of at least 0.77.

The assays may involve determining the methylation status of each CpG in a set of test CGs selected from the panel of CpGs identified at nucleotide positions 61 to 62 in SEQ ID NOs 1 to 40,753, wherein the set of CpGs comprises at least 2000 CpGs selected from the CpGs identified in SEQ ID NOs 1 to 40,753, preferably wherein the assay is characterised as having an AUC of at least 0.81.

The assays may involve determining the methylation status of each CpG in a set of test CGs selected from the panel of CpGs identified at nucleotide positions 61 to 62 in SEQ ID NOs 1 to 40,753, wherein the set of CpGs comprises at least 10,000 CpGs selected from the CpGs identified in SEQ ID NOs 1 to 40,753, preferably wherein the assay is characterised as having an AUC of at least 0.84.

The assays may involve determining the methylation status of each CpG in a set of test CGs selected from the panel of CpGs identified at nucleotide positions 61 to 62 in SEQ ID NOs 1 to 40,753, wherein the set of CpGs comprises at least 40,753 CpGs selected from the CpGs identified at nucleotide positions 61 to 62 in SEQ ID NOs 1 to 40,753, preferably wherein the assay is characterised as having an AUC of at least 0.85.

In any of the assays described herein, the predicting of the presence or development of breast cancer in an individual is based on the breast cancer index value of an individual. In any of the assays described herein, the predicting of the presence or development of breast cancer in an individual is based on the WID-BC-index value of an individual.

A breast cancer index value provides a value that indicates a “likelihood” or “risk” of any of the assays of the invention correctly predicting the presence or development of breast cancer in an individual. In the context of the present invention, “likelihood” and “risk” may be used synonymously with each other. In any of the assays described herein, the step of predicting the presence or development of breast cancer in an individual based on a breast cancer index value involves the application of threshold values. Threshold values can provide an indication of an individual's risk of harbouring breast cancer or of breast cancer development. For example, breast cancer index values may indicate at least a low risk, a moderate risk, and/or a high risk of predicting the presence or development of breast cancer.

Any references herein to sequences, genomic sequences and/or genomic coordinates are derived based upon Homo sapiens (human) genome assembly GRCh37 (hg19). The skilled person would understand variations in the nucleotide sequences of any given sequence, may exist due to sequencing errors and/or variation between individuals.

The assay of the invention represents a ‘prediction’ because any cancer index value (WID-BC-Index) derived in accordance with the invention is unlikely to be capable of diagnosing every individual as having or not having cancer with 100% specificity and 100% sensitivity. Rather, depending on the cancer index cutpoint threshold applied by the user for positively predicting the presence of cancer in an individual, the false positive and false negative rate will vary. In other words, the inventors have discovered that the assays of the invention can achieve variable levels of sensitivity and specificity for predicting the presence or development of breast cancer, as defined by receiver operating characteristics, depending on the cancer index cutpoint threshold chosen and applied by the user. Such sensitivity and specificity can be seen from the data disclosed herein to be achievable at high proportions, demonstrating accurate and statistically-significant discriminatory capability.

Similarly, cancer index values which have been pre-determined to correlate with specific breast cancer phenotypes, such as the presence of cancer, have been defined with a high level of statistical accuracy as explained further herein.

Predicting the ‘development’ of breast cancer in the context of the invention may refer to assessing whether an individual is likely or unlikely to develop breast cancer. Cells sampled from these tissues/anatomical sites can act as a surrogate for breast cells that may transform to cancer. Predicting the development of breast cancer in accordance with the assays of the invention may refer to assessing an increased or decreased likelihood of breast cancer development. Predicting the development of cancer in accordance with the assays of the invention may refer to assessing progression or worsening of a pre-existing cancer lesion in an individual. Predicting of the development of cancer in accordance with the assays of the invention may refer to predicting the likelihood of recurrence of cancer.

In any of the assays described herein, the step of assessing the presence or development of breast cancer in an individual based on a cancer index value may involve the application of a threshold value. Threshold values can provide a risk-based indication of an individual's breast cancer status, whether that is breast cancer positive, or breast cancer negative. Threshold values can also provide a means for identifying whether the cancer index value is intermediate between a breast cancer positive value and a breast cancer negative value. As explained herein, the breast cancer index value may be dynamic and subject to change depending upon genetic and/or environmental factors. Accordingly, the cancer index value may provide a means for assessing and monitoring cancer development. Breast cancer index values may therefore indicate at least a low risk or a high risk that the individual has a breast cancer positive status or has a status that is indicative of the development of breast cancer. If the cancer index value of an individual is determined by the assays of the invention at two or more time points, an increase or decrease in the individual's cancer index value may indicate an increased or decreased risk of the individual having or developing breast cancer.

Throughout the disclosure herein the terms “threshold value”, “cutpoint”, and “cutpoint threshold” are to be considered synonymous and interchangeable.

As explained further herein any assay of the invention is an assay for predicting the presence or development of breast cancer in an individual. The types of breast cancer are set out further herein. As explained further herein, the assays of the invention provide means for assessing whether an individual is at risk of having or developing breast cancer based on specific cutpoint thresholds. Such risk assessments can be provided with a high degree of confidence based on the statistical parameters which characterise the assay. Thus in any of the assays described herein involving cancer index cutpoint thresholds, the cutpoint threshold may be used for risk assessment purposes. Equally, in any of the assays described herein involving cancer index cutpoint thresholds, the cutpoint threshold value may be used to specify whether or not an individual has breast cancer as a pure diagnostic test. Again, such diagnostic tests can be provided with a high degree of confidence based on the statistical parameters which characterise the assay. Accordingly, in any assay described herein which specifies that a cancer index value for the individual is a specific value or more, or is “about” a specific value or more, the individual may be assessed as having cancer. In any assay described herein which specifies that a cancer index value for the individual is less than a specific value, or is less than “about” a specific value, the individual may be assessed as not having cancer. The term “about” is to be understood as providing a range of +/−5% of the value.

In any of the assays described herein, the predicting of the presence of breast cancer in an individual is preferably based on the WID-BC-index value.

In any of the assays described herein, wherein the WID-BC-index for the individual is about −0.235 or more, the individual is classified as having at least a low risk of harbouring breast cancer or a low risk of breast cancer development. In any of the assays described herein wherein the set of CpGs may comprise at least 500 of the CpGs defined by SEQ ID NOs 1 to 40,753 and identified at nucleotide positions 61 to 62, the sensitivity of the assay is at least 58% and the specificity of the assay is at least 44%. Wherein the set of CpGs may comprise at least the CpGs defined by SEQ ID NOs 1 to 500 and identified at nucleotide positions 61 to 62, the sensitivity of the assay is at least 85% and the specificity of the assay is at least 52%. Wherein the set of CpGs may comprise at least the CpGs defined by SEQ ID NOs 1 to 1000 and identified at nucleotide positions 61 to 62, the sensitivity of the assay is at least 88% and the specificity of the assay is at least 49%. Wherein the set of CpGs may comprise at least the CpGs defined by SEQ ID NOs 1 to 2000 and identified at nucleotide positions 61 to 62, the sensitivity of the assay is at least 94% and the specificity of the assay is at least 51%.

In any of the above described assays when a breast cancer index value threshold of −0.235 is being applied, when the WID-BC-index for the individual is about −0.235 or more, the individual may be classified as harbouring breast cancer, wherein when the WID-BC index for the individual is less than about −0.235 the individual may be classified as not harbouring breast cancer, subject to the specified sensitivity and specificity of the assay.

In any of the assays described herein, wherein the WID-BC-index for the individual is about 0.090 or more, the individual is classified as having at least a moderate risk of harbouring breast cancer or a moderate risk of breast cancer development. In any of the assays described herein wherein the set of CpGs may comprise at least 500 of the CpGs defined by SEQ ID NOs 1 to 40,753 and identified at nucleotide positions 61 to 62, the sensitivity of the assay is at least 35% and the specificity of the assay is at least 63%. Wherein the set of CpGs may comprise at least the CpGs defined by SEQ ID NOs 1 to 500 and identified at nucleotide positions 61 to 62, the sensitivity of the assay is at least 63% and the specificity of the assay is at least 69%. Wherein the set of CpGs may comprise at least the CpGs defined by SEQ ID NOs 1 to 1000 and identified at nucleotide positions 61 to 62, the sensitivity of the assay is at least 68% and the specificity of the assay is at least 73%. Wherein the set of CpGs may comprise at least the CpGs defined by SEQ ID NOs 1 to 2000 and identified at nucleotide positions 61 to 62, the sensitivity of the assay is at least 69% and the specificity of the assay is at least 78%.

In any of the above described assays when a breast cancer index value threshold of 0.090 is being applied, when the WID-BC-index for the individual is about 0.090 or more, the individual may be classified as harbouring breast cancer, wherein when the WID-BC index for the individual is less than about 0.090 the individual may be classified as not harbouring breast cancer, subject to the specified sensitivity and specificity of the assay.

In any of the assays described herein, wherein the WID-BC-index for the individual is about 0.587 or more, the individual is classified as having at least a high risk of harbouring breast cancer or a high risk of breast cancer development. In any of the assays described herein wherein the set of CpGs may comprise at least 500 of the CpGs defined by SEQ ID NOs 1 to 40,753 and identified at nucleotide positions 61 to 62, the sensitivity of the assay is at least 24% and the specificity of the assay is at least 84%. Wherein the set of CpGs may comprise at least the CpGs defined by SEQ ID NOs 1 to 500 and identified at nucleotide positions 61 to 62, the sensitivity of the assay is at least 26% and the specificity of the assay is at least 93%. Wherein the set of CpGs may comprise at least the CpGs defined by SEQ ID NOs 1 to 1000 and identified at nucleotide positions 61 to 62, the sensitivity of the assay is at least 29% and the specificity of the assay is at least 95%. Wherein the set of CpGs may comprise at least the CpGs defined by SEQ ID NOs 1 to 2000 and identified at nucleotide positions 61 to 62, the sensitivity of the assay is at least 33% and the specificity of the assay is at least 94%.

In any of the above described assays when a breast cancer index value threshold of 0.587 is being applied, when the WID-BC-index for the individual is about 0.587 or more, the individual may be classified as harbouring breast cancer, wherein when the WID-BC index for the individual is less than about 0.587 the individual may be classified as not harbouring breast cancer, subject to the specified sensitivity and specificity of the assay.

Assays according to the present invention provide a statistically robust pool of CpGs whose methylation status can be determined to provide a reliable prediction of the presence or development of breast cancer in an individual. As exemplified by the data described herein, the pool of CpGs identified by the inventors can be used in an assay of the invention having an AUC of 0.6 or more. Furthermore, subsets of the provided pool of CpGs can be assayed according to the present invention thus enabling stratification of individuals according to their risk of harbouring breast cancer or breast cancer development with statistically robust sensitivity and specificity, as determined by receiver operating characteristics.

WID-BC-index thresholds applied to patient data provided in the exemplary embodiments of the invention in the Examples herein show that low, moderate and high risk thresholds achieve desirable levels of sensitivity and specificity (see FIG. 18). For example, in the exemplary patient cohort, a low risk threshold of −0.235 captures 50% of the cohort in which 94% of all breast cancers arise. For example, in the exemplary patient cohort, a moderate risk threshold of 0.090 captures 20% of the cohort in which 78% of all breast cancers arise. For example, in the exemplary patient cohort, a high risk threshold of 0.090 captures 3% of the cohort in which 34% of all breast cancers arise.

In any of the assays described herein, the sensitivity and specificity of WID-BC-index threshold values vary depending on the number of CpGs comprised within the set, and specifically what CpGs are comprised within the set. Tables 4, 5 and 6 set out the out the AUC, sensitivity and specificity of the assays described herein depending on the number of CpGs comprised within the set, and specifically what CpGs are comprised within the set.

### Biological Samples

The assays described herein may be performed on samples of any suitable biological material. Preferably, any of the assays described herein for predicting the presence or development of breast cancer in an individual comprises providing a sample which has been taken from the individual. Preferably the individual is a woman.

In any of the assays described herein, the assay may or may not encompass the step of obtaining the sample from the individual. In assays which do not encompass the step of obtaining the sample from the individual, a sample which has previously been obtained from the individual is provided. The sample may be provided directly from the individual for analysis or may be derived from stored material, e.g. frozen, preserved, fixed or cryopreserved material.

In any of the assays described herein, the sample may be self-collected or collected by any suitable medical professional.

In any of the assays described herein, the sample from the individual may be a sample from an anatomical site other than the breast, such as a cervical, vaginal or preferably a cervicovaginal smear. In any of the assays described herein, the sample from the individual may be a sample from the breast.

Samples of biological material may include biopsy samples, solid tissue samples, aspirates such as nipple fluid aspirate, samples of biological fluids, blood, serum, plasma, peripheral blood cells, cerebrospinal fluid, urine, fine needle aspirate, saliva, sputum, breast or other hormone dependent tissue, breast milk, bone marrow, skin, samples derived from an organ comprising epithelial cells or other tissue derived from the ectoderm, vaginal fluid etc.

Samples of biological material are of preferably nipple fluid aspirate, cervical, vaginal, cervicovaginal, buccal or breast tissue origin.

Tissue scrapes may include biological material from e.g. buccal, oesophageal, bladder, vaginal, urethral or cervical scrapes.

Biopsy or other samples may be taken from any organ or tissue where a classification or prediction is desired in accordance with the methods described herein. For example, biopsy or other samples may be taken from the skin, buccal cavity, nasal cavity, salivary gland, larynx, pharynx, trachea, lung, oesophagus, stomach, small intestine, large intestine, colon, rectum, kidney, liver, bladder, heart, pancreas, gall bladder, bile duct, spleen, thymus, lymph node, thyroid gland, pituitary gland, bone, brain, breast, ovary, uterus, endometrium, cervix, vagina, vulva, testicle, penis, prostate gland.

In any of the assays described herein, the sample may particularly be derived from the cervix, the vagina, the buccal area, blood and/or urine. The sample is preferably a cervical liquid-based cytology sample, and more preferably a cervical smear sample.

Any of the assays described herein, the sample may comprise cells. The sample may comprise genetic material such as DNA and/or RNA.

Any of the assays described herein may involve providing a biological sample from the patient as the source of patient DNA for methylation analysis.

Any of the assays described herein may involve obtaining patient DNA from a biological sample which has previously been obtained from the patient.

Any of the assays described herein may involve obtaining a biological sample from the patient as the source of patient DNA for methylation analysis. The sample may be self-collected or collected by any suitable medical professional. Procedures for obtaining a biological sample from the patient may be non-invasive, such as collecting cells from urine. Alternatively, invasive procedures such as biopsy may be used.

Methods for sample isolation and for the subsequent extraction and isolation of DNA from such cell or tissue samples in preparation for assessing DNA methylation, are well known to those skilled in the art. In the context of the assays or methods described herein, the entirety of a sample may be used, or alternatively cells may be concentrated or cell types may be fractionated in order to only apply subsets of one or more cell types to the present assays or methods. Any suitable methods of concentration or fractionation may be used.

### Epithelial and Fat Cell Proportion in a Sample and Non-Fat Cell Differentiation Characteristics

The assays described herein may also comprise determining proportions of cell types within a sample which has been taken from an individual. The proportion of cell types may further enable prediction of the presence or development of breast cancer in an individual.

Determining the proportion of cells in any of the assays described herein may comprise utilisation of any suitable technique known in the art for determining cell identity and thus proportion of cells in a sample which has been taken from an individual. The determining the proportion of cells may involve genetic or epigenetic analysis. The determining the proportion of epithelial and/or fat cells may comprise determining cellular characteristics by gene expression profiling, non-coding RNA profiling, epigenome profiling, DNA methylation profiling, deriving a WID-BC-Index and/or immunohistochemistry. The determining the proportion of cells may involve comparing any one or more of said cellular characteristics with other specific cell types or reference datasets in order to robustly identify epithelial and/or fat cells in the sample. The determining the proportion of epithelial and/or fat cells may involve DNA methylation analysis, which may comprise comparison with reference DNA methylation profiles for specific cell types. The determining the proportion of cells may involve the use of EpiDISH and/or HEpiDISH.

Any of the assays described herein may comprise determining in the sample from the individual the proportion of epithelial cells and/or determining in the sample from the individual the proportion of fat cells.

High levels of epithelial cells within a sample taken from an individual may indicate an increased risk of breast cancer in the individual. Low levels of fat cells within a sample taken from an individual may indicate an increased risk of breast cancer in the individual. High levels of epithelial cells and low levels of fat cells within a sample taken from an individual may indicate an increased risk of breast cancer in the individual.

The present inventors have shown that increased epithelial cell proportion and decreased fat cell proportion in a sample taken from an individual can associate with at least a moderate risk of harbouring breast cancer or at least a moderate risk of breast cancer development as determined by derivation of a breast cancer index value in the individual. Particularly, the inventors have shown that the proportion of epithelial and fat cells in a sample taken from an individual may change. Fat cells and epithelial cells in the context of the assays disclosed herein may change with or without prior treatment being administered to the individual. In the assays and methods described herein, epithelial and/or fat cell proportion may be monitored for changes, particularly in response to one or more treatments. Fat cell and epithelial cell proportion in samples obtained from an individual, particularly sample of cervical and breast tissue origin, may reflect changes in breast cancer index according to any of the methods described herein. Thus, fat cell and epithelial cell proportion may equally represent an assay for predicting the presence or development of breast cancer in an individual and/or monitoring the risk of an individual harbouring breast cancer or of breast cancer development, in a likewise manner to the assays for determining a breast cancer index value in a sample from an individual described herein.

The assays described herein may comprise determining in the sample from the individual differentiation characteristics of non-fat cells. In combination with the breast cancer index values described herein, the differentiation of non-fat cells to fat cells may further enable prediction of the presence or development of breast cancer in an individual. “Differentiation characteristics” in the context of the present invention refers to cellular identity, as defined by any one or more cellular characteristics such as the cell's genomic or epigenomic characteristics. Particularly, the determining of differentiation characteristics may comprise comparing the characteristics of non-fat cells in the sample to characteristics of fat cells in order to determine if non-fat cells within the sample are undergoing differentiation to fat cells.

Determining differentiation characteristics of non-fat cells to fat cells in any of the assays described herein may comprise utilisation of any suitable technique known in the art for determining cell differentiation characteristics. The determining differentiation characteristics of non-fat cells involve genetic or epigenetic analysis. The determining differentiation characteristics of non-fat cells in the sample may comprise determining the non-fat cell characteristics by gene expression profiling, non-coding RNA profiling, epigenome profiling, DNA methylation profiling, deriving a WID-BC-Index and/or immunohistochemistry. The determining differentiation characteristics of the non-fat cells may involve comparing any one or more of said cellular characteristics with characteristics of fat cells or fat cell reference data e.g. publically available ENCODE data. The determining differentiation characteristics of non-fat cells may involve the detection of lipids in the sample by any suitable method. The determining differentiation characteristics of non-fat cells may involve DNA methylation analysis, which may comprise comparison with reference DNA methylation profiles for specific fat cell types. The determining differentiation characteristics of non-fat cells may involve RT-PCR based methods for detection of known genetic markers of fat cells. The determining the proportion of cells may involve the use of EpiDISH and/or HEpiDISH.

Preferably, in any of the assays described herein, the sample derived from the individual for determining changes in epithelial cell proportion, fat cell proportion and/or differentiation characteristics of non-fat cells is a sample from the breast.

### Types of Cancers

The methods described herein may be applied to any breast cancer.

The breast cancer may be a ductal carcinoma in situ or an invasive ductal carcinoma such as tubular type invasive ductal carcinoma (IDC), medullary type IDC, mucinous type IDC, papillary type IDC or cribriform type IDC.

The breast cancer may be an invasive carcinoma such as a pleomorphic carcinoma, carcinoma with osteoclast giant cells, carcinoma with choriocarcinoma features, carcinoma with melanotic features. The invasive breast carcinoma may be an invasive lobular carcinoma, tubular carcinoma, invasive cribriform carcinoma, medullary carcinoma, mucinous carcinoma and other tumours with abundant mucin such as mucinous carcinoma, cystadenocarcinoma and columna cell mucinous carcinoma, signet ring cell carcinoma. The invasive breast carcinoma may be a neuroendocrine tumour such as solid neuroendocrine carcinoma (carcinoid of the breast), atypical acarcinoid tumour, small cell/oat cell carcinoma, large cell neuroendocrine carcinoma. The invasive breast carcinoma may be an invasive papillary carcinoma, invasive micropapillary carcinoma, apocrine carcinoma, metaplastic carcinomas such as pure epithelial metaplastic carcinomas including squamous cell carcinoma, adenocarcinoma with spindle cell metaplasia, adenosquamous carcinoma, mucoepidermoid carcinoma, mixed epithelial/mesenchymal metaplastic carcinomas, matrix-producing carcinoma, spindle cell carcinoma, carcinosarcoma, squamous cell carcinoma of mammary origin, metaplastic carcinoma with osteoclastic giant cells. The invasive breast carcinoma may be a lipid-rich carcinoma, secretory carcinoma, oncocytic carcinoma, adenoid cystic carcinoma, acinic cell carcinoma, glycogen-rich clear cell carcinoma, sebaceous carcinoma, inflammatory carcinoma, bilateral breast carcinoma.

The breast cancer may be a mesenchymal breast tumour. The mesenchymal tumour may include sarcoma. The mesenchymal breast tumour may be a hemangioma, angiomatosis, Hemangiopericytoma, Pseudoangiomatous stromal hyperplasia, Myofibroblastoma, Fibromatosis (aggressive), Inflammatory myofibroblastic tumor, Lipoma Angiolipoma, Granular cell tumour, Neurofibroma, Schwannoma, Angiosarcoma, Liposarcoma, Rhabdomyosarcoma, Osteosarcoma, Leiomyoma, Leiomyosarcoma.

The breast cancer may be a malignant lymphoma such as Non-Hodgkin lymphoma.

The breast cancer may be a metastatic tumour in which the primary lesion originated in a tissue other than the breast.

The breast cancer may be a precursor breast cancer lesion. The precursor breast cancer lesion may be a Lobular neoplasia, lobular carcinoma in situ, Intraductal proliferative lesions, Usual ductal hyperplasia, Flat epithelial hyperplasia, Atypical ductal hyperplasia, Ductal carcinoma in situ, Microinvasive carcinoma, Intraductal papillary neoplasms, Central papilloma, Peripheral papilloma, Atypical papilloma, Intraductal papillary carcinoma, Intracystic papillary carcinoma.

The breast cancer may be a myoepithelial breast cancer lesion. The myoepithelial breast cancer lesion be myoepitheliosis, adenomyoepithelial adenosis, adenomyoepithelioma, malignant myoepithelioma.

The breast cancer may be a fibroepithelial breast tumour. The fibroepithelial breast tumour may be a fibroadenoma, phyllodes tumour, periductal stromal sarcoma, mammary hamartoma.

The breast cancer may be Paget's disease of the nipple.

### Methods of Treatment and Diagnosis

The invention also encompasses the performance of one or more treatment steps following a positive classification of cancer or prediction of cancer development based on any of the methods described herein.

The invention also encompasses the performance of one or more treatment steps following a negative classification of cancer or prediction of cancer development based on any of the methods described herein. Said treatments may be considered “risk prevention” or “prophylactic” treatments.

The invention also encompasses the performance of one or more treatment steps following a negative classification or cancer or prediction of cancer development based on any of the methods described herein, in an individual that harbours one or more mutations that predispose the individual to an increased risk of developing breast cancer.

The invention thus encompasses a method of treating breast cancer in an individual comprising:
a. predicting the presence or development of breast cancer in an individual comprising any one of the assays described herein;
b. stratifying the individual according to their risk of harbouring breast cancer or according to their risk of breast cancer development; and
c. administering one or more treatments to the individual based on their risk stratification.

The invention thus encompasses a method of treating breast cancer in an individual comprising:
a. predicting the presence or development of breast cancer in an individual comprising:
   i. providing a sample which has been taken from the individual;
   ii. determining in DNA in the sample the methylation status of each CpG in a set of test CpGs selected from the panel of CpGs identified at nucleotide positions 61 to 62 in SEQ ID NOs 1 to 40,753;
   iii. deriving a breast cancer index value based on the methylation status of the test CpGs; and
   iv. predicting the presence or development of breast cancer in the individual based on the breast cancer index value;
      wherein the assay is characterised as having an area under the curve (AUC) of 0.6 or more as determined by receiver operating characteristics (ROC);
b. stratifying the individual according to their risk of harbouring breast cancer or according to their risk of breast cancer development; and
c. administering one or more treatments to the individual based on their risk stratification.

The invention thus encompasses a method of treating breast cancer in an individual comprising:
a. predicting the presence or development of breast cancer in an individual comprising:
   i. providing a sample which has been taken from the individual;
   ii. determining in DNA in the sample the methylation status of each CpG in a set of test CpGs comprises at least 500 CpGs selected from the CpGs identified at nucleotide positions 61 to 62 in SEQ ID NOs 1 to 40,753;
   iii. deriving a breast cancer index value based on the methylation status of the test CpGs; and
   iv. predicting the presence or development of breast cancer in the individual based on the breast cancer index value;
      wherein the assay is characterised as having an area under the curve (AUC) of 0.73 or more as determined by receiver operating characteristics (ROC);
b. stratifying the individual according to their risk of harbouring breast cancer or according to their risk of breast cancer development; and
c. administering one or more treatments to the individual based on their risk stratification.

The invention thus encompasses a method of treating breast cancer in an individual comprising:
a. predicting the presence or development of breast cancer in an individual comprising:
   i. providing a sample which has been taken from the individual;
   ii. determining in DNA in the sample the methylation status of each CpG in a set of test CpGs comprises at least 1000 CpGs selected from the CpGs identified at nucleotide positions 61 to 62 in SEQ ID NOs 1 to 40,753;
   iii. deriving a breast cancer index value based on the methylation status of the test CpGs; and
   iv. predicting the presence or development of breast cancer in the individual based on the breast cancer index value;
      wherein the assay is characterised as having an area under the curve (AUC) of 0.77 or more as determined by receiver operating characteristics (ROC);
b. stratifying the individual according to their risk of harbouring breast cancer or according to their risk of breast cancer development; and
c. administering one or more treatments to the individual based on their risk stratification.

The invention thus encompasses a method of treating breast cancer in an individual comprising:
a. predicting the presence or development of breast cancer in an individual comprising:
   i. providing a sample which has been taken from the individual;
   ii. determining in DNA in the sample the methylation status of each CpG in a set of test CpGs comprises at least 2000 CpGs selected from the CpGs identified at nucleotide positions 61 to 62 in SEQ ID NOs 1 to 40,753;
   iii. deriving a breast cancer index value based on the methylation status of the test CpGs; and
   iv. predicting the presence or development of breast cancer in the individual based on the breast cancer index value;
      wherein the assay is characterised as having an area under the curve (AUC) of 0.81 or more as determined by receiver operating characteristics (ROC);
b. stratifying the individual according to their risk of harbouring breast cancer or according to their risk of breast cancer development; and
c. administering one or more treatments to the individual based on their risk stratification.

In any of the methods of treatment encompassed by the invention, the step of predicting the presence or development of breast cancer in an individual may involve determining in DNA derived from cells in the sample the methylation status of in any set of test CpGs according to the assays of the invention.

In any of the methods of treatment encompassed by the invention, the step of predicting the presence or development of breast cancer in an individual may involve deriving a WID-BC-index value.

In any of the methods of treatment encompassed by the invention, the step of predicting the presence or development of breast cancer in an individual may involve the use of any one of the arrays described herein.

In any of the methods of treatment encompassed by the invention, the step of stratifying the individual may involve applying any one of the thresholds according to any one of the assays of the invention described herein.

The step of administering one or more treatments may comprise different treatment steps depending on the stratification of an individual on the basis of their risk of harbouring breast cancer or on the basis of risk of breast cancer development. Particularly the amount of an invasiveness of the treatments administered may vary dependent on the stratification of an individual on the basis of their risk of harbouring breast cancer or on the basis of their risk of breast cancer development. The treatments administered to the individual may comprise any treatments considered suitable by a person skilled in the art. For example, wherein the individual is stratified as low risk and the individual is subjected to intensified screening. The intensified screening may comprise one or more mammography scans and/or breast MRI scans.

Wherein the individual is stratified as moderate risk and the individual is subjected to intensified screening and/or administration of one or more suitable doses of one or more of Mifepristone, Aromatase inhibitors, Denosumab, “selective estrogen modulators” (SERMs) and “selective progesterone receptor modulators” (SPRMs). SERMs may include Anordin, Bazedoxifene, Broparestrol, Clomifene, Cyclofenil, Lasofoxifene, Ormeloxifene, Ospemifene, Raloxifene, Tamoxifen. Preferably, the SERMs include Tamoxifen, Bazedoxifene and Raloxifene. Preferably, the SPRMs include Mifepristone, Ulipristal, Asoprisnil, Proellex, Onapristone, Asoprisnil and Lonaprisan. The intensified screening may comprise one or more mammography scans and/or breast MRI scans. Any of the methods of treatment described herein, wherein the individual is stratified as “moderate” risk, the one or more treatments to the individual may function as ‘preventative’ treatments. Particularly, any one of the treatments described herein may be administered to an individual stratified as at least moderate risk as a measure of preventing manifestation of breast cancer in said individual.

Wherein the individual is stratified as high risk and the individual is subjected to intensified screening and/or administration of one or more suitable doses of one or more of Mifeprestone, Aromatase inhibitors, Denosumab, SERMS, SPRMs and/or bilateral mastectomy. SERMs may include Anordin, Bazedoxifene, Broparestrol, Clomifene, Cyclofenil, Lasofoxifene, Ormeloxifene, Ospemifene, Raloxifene, Tamoxifen. Preferably, the SERMs include Tamoxifen, Bazedoxifene and Raloxifene. Preferably, the SPRMs include Mifepristone, Ulipristal, Asoprisnil, Proellex, Onapristone, Asoprisnil and Lonaprisan.

In any one of the methods of treatment described herein, the method may further comprise genetic and/or expression profiling of any panel of genes known in the art as being associated with breast cancer. For example, the methods described herein may further comprise genetic and/or expression profiling of any one or more of the genes comprised within the MammaPrint™ test (Cardoso et al, N Engl J Med, 2016; 375:717-729). For any panel of genes known in the art as being associated with breast cancer, the skilled person would be aware of what genetic and/or expression profiles would be considered to be abnormal. Furthermore, the skilled person would be aware of treatments in the art that are known to be efficacious with respect to specific abnormalities observed in profiling any panel of genes known in the art as being associated with breast cancer. For example, upon observing one or more mutations in one or both of the BRCA1 and BRCA2 genes the skilled person would consider administering platinum-based treatments to the individual.

Wherein the individual is predicted as not harbouring breast cancer, the individual may be subjected to risk-prevention treatments. Particularly, for example, if the individual has one or more genetic mutations that predispose an individual to an increased risk of developing breast cancer, the individual may be subjected to risk-prevention treatments. Risk-prevention treatments may comprise any suitable treatment. For example, a risk prevention treatment may be administering one or more doses of mifepristone. In any of the methods described herein, the individual may not harbour breast cancer, but may harbour one or more genetic mutations that pre-dispose the individual to breast cancer such as one or more mutations in the BRCA genes. Other mutations may include any mutations in the art that are considered to pre-dispose individuals to breast cancer. In any of the methods of treatment described herein, the individual may not harbour breast cancer but may harbour one or more genetic mutations that pre-dispose the individual to breast cancer, and this individual may be subjected to any of the methods of monitoring described herein. For example, in any of the methods described herein, the individual does not harbour breast cancer and harbours one or more mutations that predispose the individual to an increased risk of developing breast cancer, and wherein one or more treatments administered to the individual comprises one or more doses of mifepristone. In any of the methods described herein, the individual does not harbour breast cancer and harbours one or more mutations in a BRCA gene, and wherein one or more treatments administered to the individual comprises one or more doses of mifepristone

Other exemplary treatments comprise one or more surgical procedures, one or more chemotherapeutic agents, one or more cytotoxic chemotherapeutic agents one or more radiotherapeutic agents, one or more immunotherapeutic agents, one or more biological therapeutics, one or more anti-hormonal treatments or any combination of the above following a positive diagnosis of cancer.

Cancer treatments may be administered to an individual harbouring breast cancer or at risk of breast cancer development, in an amount sufficient to prevent, treat, cure, alleviate or partially arrest breast cancer or one or more of its symptoms. Such treatments may result in a decrease in severity, and/or decreased breast cancer index value, of breast cancer symptoms, or an increase in frequency or duration of symptom-free periods. A treatment amount adequate to accomplish this is defined as “therapeutically effective amount”. Effective amounts for a given purpose will depend on the severity of breast cancer and/or the individual's breast cancer index value as well as the weight and general state of the individual. As used herein, the term “individual” includes any human, preferably wherein the human is a woman. As used herein, “treatment” is to be considered synonymous with “therapeutic agent”.

The following therapeutic agents may be administered to an individual based on their breast cancer risk alone or in combination with any other treatment described herein. The therapeutic agent may be directly attached, for example by chemical conjugation, to an antibody. Methods of conjugating agents or labels to an antibody are known in the art. For example, carbodiimide conjugation (Bauminger & Wilchek (1980) Methods Enzymol. 70, 151-159) may be used to conjugate a variety of agents, including doxorubicin, to antibodies or peptides. The water-soluble carbodiimide, 1-ethyl-3-(3-dimethylaminopropyl) carbodiimide (EDC) is particularly useful for conjugating a functional moiety to a binding moiety. Other methods for conjugating a moiety to antibodies can also be used. For example, sodium periodate oxidation followed by reductive alkylation of appropriate reactants can be used, as can glutaraldehyde cross-linking. However, it is recognised that, regardless of which method of producing a conjugate of the invention is selected, a determination must be made that the antibody maintains its targeting ability and that the functional moiety maintains its relevant function.

A cytotoxic moiety may be directly and/or indirectly cytotoxic. By “directly cytotoxic” it is meant that the moiety is one which on its own is cytotoxic. By “indirectly cytotoxic” it is meant that the moiety is one which, although is not itself cytotoxic, can induce cytotoxicity, for example by its action on a further molecule or by further action on it. The cytotoxic moiety may be cytotoxic only when intracellular and is preferably not cytotoxic when extracellular.

Cytotoxic chemotherapeutic agents are well known in the art. Cytotoxic chemotherapeutic agents, such as anticancer agents, include: alkylating agents including nitrogen mustards such as mechlorethamine (HN2), cyclophosphamide, ifosfamide, melphalan (L-sarcolysin) and chlorambucil; ethylenimines and methylmelamines such as hexamethylmelamine, thiotepa; alkyl sulphonates such as busulfan; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); Antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin). Natural Products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; and biological response modifiers such as interferon alphenomes. Miscellaneous agents including platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MIH); and adrenocortical suppressant such as mitotane (o,p′-DDD) and aminoglutethimide; taxol and analogues/derivatives; and hormone agonists/antagonists such as flutamide and tamoxifen.

A cytotoxic chemotherapeutic agent may be a cytotoxic peptide or polypeptide moiety which leads to cell death. Cytotoxic peptide and polypeptide moieties are well known in the art and include, for example, ricin, abrin, Pseudomonas exotoxin, tissue factor and the like. Methods for linking them to targeting moieties such as antibodies are also known in the art. Other ribosome inactivating proteins are described as cytotoxic agents in WO 96/06641. Pseudomonas exotoxin may also be used as the cytotoxic polypeptide. Certain cytokines, such as TNFα and IL-2, may also be useful as cytotoxic agents.

Certain radioactive atoms may also be cytotoxic if delivered in sufficient doses. Radiotherapeutic agents may comprise a radioactive atom which, in use, delivers a sufficient quantity of radioactivity to the target site so as to be cytotoxic. Suitable radioactive atoms include phosphorus-32, iodine-125, iodine-131, indium-111, rhenium-186, rhenium-188 or yttrium-90, or any other isotope which emits enough energy to destroy neighbouring cells, organelles or nucleic acid. Preferably, the isotopes and density of radioactive atoms in the agents of the invention are such that a dose of more than 4000 cGy (preferably at least 6000, 8000 or 10000 cGy) is delivered to the target site and, preferably, to the cells at the target site and their organelles, particularly the nucleus.

The radioactive atom may be attached to an antibody, antigen-binding fragment, variant, fusion or derivative thereof in known ways. For example, EDTA or another chelating agent may be attached to the binding moiety and used to attach 111In or 90Y. Tyrosine residues may be directly labelled with 125I or 131I.

A cytotoxic chemotherapeutic agent may be a suitable indirectly-cytotoxic polypeptide. In a particularly preferred embodiment, the indirectly cytotoxic polypeptide is a polypeptide which has enzymatic activity and can convert a non-toxic and/or relatively non-toxic prodrug into a cytotoxic drug. With antibodies, this type of system is often referred to as ADEPT (Antibody-Directed Enzyme Prodrug Therapy). The system requires that the antibody locates the enzymatic portion to the desired site in the body of the patient and after allowing time for the enzyme to localise at the site, administering a prodrug which is a substrate for the enzyme, the end product of the catalysis being a cytotoxic compound. The object of the approach is to maximise the concentration of drug at the desired site and to minimise the concentration of drug in normal tissues. In a preferred embodiment, the cytotoxic moiety is capable of converting a non-cytotoxic prodrug into a cytotoxic drug.

### Method of Monitoring

The invention also provides methods of monitoring the risk of the presence or development of breast cancer in an individual.

“Monitoring” in the context of the present invention may refer to longitudinal assessment of an individual's risk of harbouring breast cancer or risk of breast cancer development. This longitudinal assessment may be carried out according to the assays of the invention described herein. This longitudinal assessment may involve performance of the assays of the invention described herein to predict the presence or development of breast cancer in an individual at more than one time point over the course of an undetermined time window. The time window may be any period of time whilst the individual is still living. The time window may persist for the lifetime of the individual. The time window may persist until the individual's risk of harbouring breast cancer or risk of breast cancer development falls below a certain level. The level may be a particular breast cancer index value e.g. a WID-BC-index value.

The invention thus encompasses a method of monitoring the risk of an individual harbouring breast cancer or of monitoring the risk of breast cancer development, the method comprising:
a. predicting the presence or breast cancer in an individual or predicting breast cancer development in an individual by performing any one of the assays described herein at a first time point;
b. predicting the presence of breast cancer in the individual or predicting breast cancer development in the individual by performing any one of the assays described herein at one or more further time points; and
c. monitoring any change in the individual's risk between time points.

The invention also encompasses a method of monitoring the risk of an individual harbouring breast cancer or of monitoring the risk of breast cancer development, the method comprising:
a. predicting the presence or breast cancer in an individual or predicting breast cancer development in an individual by performing an assay at a first time point, comprising:
   i. providing a sample which has been taken from the individual;
   ii. determining in DNA in the sample the methylation status of each CpG in a set of test CpGs selected from the panel of CpGs identified at nucleotide positions 61 to 62 in SEQ ID NOs 1 to 40,753;
   iii. deriving a breast cancer index value based on the methylation status of the test CpGs; and
   iv. predicting the presence or development of breast cancer in the individual based on the breast cancer index value;
      wherein the assay is characterised as having an area under the curve (AUC) of 0.6 or more as determined by receiver operating characteristics (ROC);
b. predicting the presence of breast cancer in the individual or predicting breast cancer development in the individual by performing any one of the assays described herein at one or more further time points; and
c. monitoring any change in the individual's risk between time points.

The invention also encompasses a method of monitoring the risk of an individual harbouring breast cancer or of monitoring the risk of breast cancer development, the method comprising:
a. predicting the presence or breast cancer in an individual or predicting breast cancer development in an individual by performing an assay at a first time point, comprising:
   i. providing a sample which has been taken from the individual;
   ii. determining in DNA in the sample the methylation status of each CpG in a set of test CpGs comprises at least 500 CpGs selected from the CpGs identified at nucleotide positions 61 to 62 in SEQ ID NOs 1 to 40,753;
   iii. deriving a breast cancer index value based on the methylation status of the test CpGs; and
   iv. predicting the presence or development of breast cancer in the individual based on the breast cancer index value;
      wherein the assay is characterised as having an area under the curve (AUC) of 0.73 or more as determined by receiver operating characteristics (ROC);
b. predicting the presence of breast cancer in the individual or predicting breast cancer development in the individual by performing any one of the assays described herein at one or more further time points; and
c. monitoring any change in the individual's risk between time points.

The invention also encompasses a method of monitoring the risk of an individual harbouring breast cancer or of monitoring the risk of breast cancer development, the method comprising:
a. predicting the presence or breast cancer in an individual or predicting breast cancer development in an individual by performing an assay at a first time point, comprising:
   i. providing a sample which has been taken from the individual;
   ii. determining in DNA in the sample the methylation status of each CpG in a set of test CpGs comprises at least 1000 CpGs selected from the CpGs identified at nucleotide positions 61 to 62 in SEQ ID NOs 1 to 40,753;
   iii. deriving a breast cancer index value based on the methylation status of the test CpGs; and
   iv. predicting the presence or development of breast cancer in the individual based on the breast cancer index value;
      wherein the assay is characterised as having an area under the curve (AUC) of 0.77 or more as determined by receiver operating characteristics (ROC);
b. predicting the presence of breast cancer in the individual or predicting breast cancer development in the individual by performing any one of the assays described herein at one or more further time points; and
c. monitoring any change in the individual's risk between time points.

The invention also encompasses a method of monitoring the risk of an individual harbouring breast cancer or of monitoring the risk of breast cancer development, the method comprising:
a. predicting the presence or breast cancer in an individual or predicting breast cancer development in an individual by performing an assay at a first time point, comprising:
   i. providing a sample which has been taken from the individual;
   ii. determining in DNA in the sample the methylation status of each CpG in a set of test CpGs comprises at least 2000 CpGs selected from the CpGs identified at nucleotide positions 61 to 62 in SEQ ID NOs 1 to 40,753;
   iii. deriving a breast cancer index value based on the methylation status of the test CpGs; and
   iv. predicting the presence or development of breast cancer in the individual based on the breast cancer index value;
      wherein the assay is characterised as having an area under the curve (AUC) of 0.81 or more as determined by receiver operating characteristics (ROC);
b. predicting the presence of breast cancer in the individual or predicting breast cancer development in the individual by performing any one of the assays described herein at one or more further time points; and
c. monitoring any change in the individual's risk between time points.

In any of the methods of monitoring encompassed by the invention, the steps of predicting the presence of breast cancer or development of breast cancer in an individual may involve determining in DNA derived in the sample the methylation status of in any set of test CpGs according to the assays of the invention.

In any of the methods of monitoring described herein, the steps of predicting the presence or development of breast cancer in an individual based on a breast cancer index value may involve the application of threshold values. Threshold values can provide an indication of an individual's risk of harbouring breast cancer or an individual's risk of breast cancer development. For example, breast cancer index values may indicate at least a low risk, a modest risk, and/or a high risk of predicting the presence or development of breast cancer. In any of the methods of monitoring encompassed by the invention, the step of predicting the presence or development of breast cancer in an individual may involve deriving a WID-BC-index value.

In any of the methods of monitoring described herein, the individual may already harbour breast cancer. The individual may not have breast cancer. The individual may not harbour breast cancer. The individual may not harbour breast cancer but may harbour one or more genetic mutations that predispose the individual to an increased risk of breast cancer development e.g. the individual may harbour one or more mutations in a BRCA gene. Other mutations may include any mutations in the art that are considered to pre-dispose individuals to breast cancer. In any of the methods of monitoring described herein, the individual may not harbour breast cancer but may harbour one or more genetic mutations that pre-dispose the individual to breast cancer, and this individual may be subjected to any of the methods of monitoring described herein in order to determine their risk of harbouring breast cancer or of breast cancer development. For example, in any of the methods described herein, the individual does not harbour breast cancer and harbours one or more mutations that predispose the individual to an increased risk of developing breast cancer, and wherein one or more treatments are administered to the individual in accordance with any of the methods of treatment described herein as a method of prophylaxis. In any of the methods described herein, the individual does not harbour breast cancer and harbours one or more mutations that predispose the individual to an increased risk of developing breast cancer, and wherein one or more treatments are administered to the individual in accordance with any of the methods of treatment described herein as a method of prophylaxis, and wherein the one or more treatments administered to the individual comprises one or more doses of SPRMs e.g. comprising one or more doses of mifepristone.

In any of the methods of monitoring described herein, depending on the risk of the presence or development of breast cancer in the individual, one or more treatments are administered to the individual according to any one of the methods of treatment encompassed by the invention and described herein. Different treatments may be administered depending on the stratification of an individual on the basis of their risk of harbouring breast cancer or on the basis of their risk of breast cancer development. The method may further comprise administration of one or more treatments according to the methods of treatment described herein.

The breast cancer index value may change between any two or more time points. Likewise, in the sample taken from the individual, epithelial cell proportion, fat cell proportion and/or differentiation status of non-fat cells may change between any two or more time points. For this reason, longitudinal monitoring of an individual's breast cancer index value could be of particular benefit to the assessment of, for example, breast cancer progression, treatment efficacy, or breast cancer efficacy.

Any of the methods described herein may comprise:
a. determining the proportion of epithelial cells in the sample from the individual between any two or more time points and assessing if the proportion changes between time points;
b. determining the proportion of fat cells in the sample from the individual between any two or more time points and assessing if the proportion changes between time points; and/or
c. determining differentiation characteristics of non-fat cells in the sample from the individual between any two or more time points and assessing if the proportion changes between time points.

In any of the methods described herein, wherein
a. an increase in the breast cancer index value and an increase in the proportion of epithelial cells; and/or
b. an increase in the breast cancer index value and a decrease in the proportion of fat cells; and/or
c. an increase in the breast cancer index value and an increase in differentiation of non-fat cells towards fat cells,

indicates a negative response to the one or more treatments. Changes may be made to the one or more treatments if a negative response is identified.

In any of the methods described herein, wherein
a. a decrease in the breast cancer index value and a decrease in the proportion of epithelial cells;
b. a decrease in the breast cancer index value and an increase in the proportion of fat cells; and/or
c. a decrease in the breast cancer index value and a decrease in differentiation of non-fat cells towards fat cells,

indicates a positive response to the one or more treatments. Changes may be made to the one or more treatments if a positive response is identified.

In any of the methods of monitoring described herein, the one or more further time points may be any suitable time point. Preferably the one or more further time points may of suitable distance apart for sufficiently frequent screening in order to predict any particularly early onset cases of presence or development of breast cancer in an individual. Preferably the one or more further time points may be of suitable distance apart for assessing the efficacy of one or more treatments. Preferably the one or more further time points may be of suitable distance apart for predicting whether an individual remains free of cancer after a successful course of treatment. The one or more further time points may be about monthly, about two monthly, about three monthly, about four monthly, about five monthly, about six monthly, about seven monthly, about eight monthly, about nine monthly, about ten monthly, about eleven monthly, about yearly, about two yearly, or more than two yearly.

In any of the methods of monitoring described herein, changes may be made to the one or more treatments wherein a positive or negative responses to the one or more treatments are observed. Treatments may be changed in accordance with the methods of treatments described herein. Treatments may particularly be changed if the individual's risk stratification, based on their breast cancer index value, changes.

In any of the methods of monitoring encompassed by the invention, the step of predicting the presence or development of breast cancer in an individual may involve the use of any one of the arrays described herein.

### Arrays and Kits

The invention also encompasses arrays capable of discriminating between methylated and non-methylated forms of CpGs as defined herein; the arrays may comprise oligonucleotide probes specific for methylated forms of CpGs as defined herein and oligonucleotide probes specific for non-methylated forms of CpGs as defined herein. In any of the arrays described herein, the array may comprise oligonucleotide probes specific for a methylated form of each CpG in a CpG panel and oligonucleotide probes specific for a non-methylated form of each CpG in the panel; wherein the panel consists of at least 500 CpGs selected from the CpGs identified in SEQ ID NOs 1 to 40,753

In some embodiments the array is not an Infinium MethylationEPIC BeadChip array or an Illumina Infinium HumanMethylation450 BeadChip array.

Separately or additionally, in some embodiments the number of CpG-specific oligonucleotide probes of the array is 482,000 or less, 480,000 or less, 450,000 or less, 440,000 or less, 430,000 or less, 420,000 or less, 410,000 or less, or 400,000 or less, 375,000 or less, 350,000 or less, 325,000 or less, 300,000 or less, 275,000 or less, 250,000 or less, 225,000 or less, 200,000 or less, 175,000 or less, 150,000 or less, 125,000 or less, 100,000 or less, 75,000 or less, 50,000 or less, 45,000 or less, 40,000 or less, 35,000 or less, 30,000 or less, 25,000 or less, 20,000 or less, 15,000 or less, 10,000 or less, 5,000 or less, 4,000 or less, 3,000 or less or 2,000 or less.

The CpG panel may comprise any set of CpGs defined in the assays of the invention described herein.

The arrays of the invention may comprise one or more oligonucleotides comprising any set of CpGs defined in the assays of the invention, wherein the one or more oligonucleotides are hybridized to corresponding oligonucleotide probes of the array.

The invention also encompasses a process for making a hybridized array described herein, comprising contacting an array according to the present invention with a group of oligonucleotides comprising any set of CpGs defined in the assays of the invention.

Any of the arrays as defined herein may be comprised in a kit. The kit may comprise any array as defined herein together with instructions for use.

The invention further encompasses the use of any of the arrays as defined herein in any of the assays for determining the methylation status of CpGs for the purposes of predicting the presence or development of breast cancer in an individual.

The invention is illustrated by the following Examples:

### EXAMPLES

Breast cancer is by far the most common cancer in females in general, and a leading cause of death in young women¹. To date, the identification of individuals with primary cancer is achieved by assessing evidence directly from the tumour (e.g. imaging or detection of cancer cell products released into the system^{3,4}). Currently available early detection strategies, such as mammography screening, suffer from low performance in young women, over-diagnosis, and decreasing attendance rates, and its benefit on mortality rates has recently been questioned⁵. Developing models which allow for a stratified breast cancer early detection and prevention strategy have proven to be challenging, and the best predictive models combining epidemiological risk factors, single nucleotide polymorphisms (SNPs) and mammographic density have only led to a Receiver Operator Characteristic (ROC) Area Under the Curve (AUC) of 0.68⁶.

In contrast, cervical cancer screening (i.e. assessing cervical smear samples) has reduced the incidence and mortality from cervical cancer by more than 50%⁷. The fact that clinician- and self-collected samples show similar performance in detecting relevant cervical lesions' is likely to further increase attendance rates.

Epigenetic (i.e. DNAme) changes have been identified in normal breast tissue adjacent to breast cancers' and could potentially serve as a surrogate for both genetic and non-genetic factors including lifestyle, reproductive and environmental exposures contributing to breast cancer development¹⁰ . A number of proof of principle studies, so far exclusively performed in blood, have demonstrated that certain DNAme changes are associated with breast cancer predisposition¹¹⁻¹⁶. Sample heterogeneity and the choice of surrogate tissue are deemed to be among the most important factors impeding clinical implementation¹⁷. Thus, we aimed to assess whether DNAme profiles derived from cervical smear samples (i.e. containing hormone sensitive epithelial cells which are capable of recording breast cancer-predisposing factors at the level of the epigenome¹⁷ and can be self-collected) are able to identify women with primary breast cancer.

We performed an epigenome-wide DNAme analysis in cervical smear samples from women who had recently been diagnosed with breast cancer, and in matched controls, and established the WID-BC-index (Women's risk IDentification for Breast Cancer index) which we further validated in buccal samples and in an independent set of cervical samples. In addition, we assessed the WID-BC-index in a larger number of different cell types as well as in breast samples, including normal breast samples from clinical trials before and after an intervention.

### Materials and Methods

### Study Design and Epidemiological Data Acquisition:

The study was conducted as part of a multi-centre study involving several recruitment sites in 5 European countries (i.e. the UK, Czech Republic, Italy, Norway and Germany) (Table 2).

TABLE 2
Overview of breast cancer cases and control cases collected
in different countries (discovery set).

| Country | Breast cancer | Control | Total |
|---|---|---|---|
| Czech | 32 | 206 | 238 |
| Germany | 36 | 2 | 38 |
| Italy | 201 | 53 | 254 |
| Norway | 0 | 132 | 132 |
| UK | 16 | 385 | 401 |
| Total | 365 | 698 | 1,063 |

Participants were aged >18 years. Prior to taking part, each prospective study volunteer was given a Participant Information Sheet as well as a Consent Form and the rationale for the study was explained. Additional resources, including an explanatory video and further online resources, were also made available. Women diagnosed with breast cancer (case) or a non-malignant benign gynaecological condition (control) were approached during outpatient hospital clinics, while women recruited as healthy volunteers from the general population (control) were approached via outreach campaigns, public engagement, and as part of cervical screening programmes. After signing an informed consent, participants completed an epidemiological questionnaire as well as a feedback form after their participation. The study itself is a sub-study of the FORECEE (4C) Programme, which has ethical approval from the UK Health Research Authority (REC 14/LO/1633) and other contributing centres.

The epidemiological survey was administered via the Qualtrics application on dedicated iPads. The survey contained questions relating to health habits, relevant risk factors, and also made enquiries as to historical health habits, as well as obtaining a thorough medical and obstetric history. Cervical samples were collected at appropriate clinical venues by trained staff and the cervical smears were carried out by a small group of research midwives or physicians with a view to establishing standard practice. Buccal samples were collected using Copan 4N6FLOQ Swabs, Thermofisher Scientific.

Biological samples were given an anonymous Participant ID Number which was assigned to the person's name in a securely stored link file. Following sample taking, an email survey was sent to each participant, enabling them to feedback with respect to the recruitment process. Women with a current diagnosis of a primary breast cancer with poor prognosis features (Grade III and/or T2/3 and/or N1/2 and/or HR-ve) and recruited prior to receiving any systemic treatment (chemo- or antihormonal or Herceptin, etc.) or surgery or radiotherapy were eligible as breast cancer cases. Controls were initially matched one-to-one with cases based on menopausal status, age (5 year age ranges where possible), and recruitment centre/country. However, due to an imbalance in recruitment of cases and controls at some centres, a number of cases were matched on age and menopausal status alone. Cancer histological data was collected post-recruitment either by clinicians directly involved in the diagnosis/treatment of the cancer cases or by a nominated data manager with access to the in-house hospital

### Cervical Smear Sample Collection

Cervical smears were taken at collaborating hospitals and recruitment centres using the ThinPrep system (Hologic Inc., cat #70098-002). Cervical cells were sampled from the cervix using a cervix brush (Rovers Medical Devices, cat #70671-001) which was rotated 5 times through 360 degrees whilst in contact with the cervix to maximise cell sampling. The brush was removed from the vagina and immersed in a ThinPrep vial containing Preserve-cyt fluid and then pushed against the bottom of the vial 10 times to facilitate release of the cells from the brush into the solution. The sample vial was sealed and stored locally at room temperature. Buccal cells were collected using two Copan 4N6FLOQ Buccal Swabs (Copan Medical Diagnostics, cat #4504C) by firmly brushing the swab head 5-6 times against the buccal mucosa of each cheek. The swabs were re-capped and left to dry out at room temperature within the sampling tube which contains a drying desiccant. 2.5 ml of venous whole blood was collected in PAX gene blood DNA tubes (BD Biosciences #761165) and stored locally at 4° C. All samples were shipped to UCL at ambient temperature.

### Breast Tissue Samples

We have analysed two independent sets of breast tissue samples. The first set contained a total of 56 breast samples from premenopausal women aged 19-54 years (FIG. 7B): normal breast tissue from 14 women who underwent cosmetic breast operations, normal breast tissue from women who underwent prophylactic mastectomies due to a BRCA1 (n=9) or a BRCA2 (n=5) mutation, and 14 women who had breast surgery due to a triple negative breast cancer and who provided both normal adjacent breast tissue as well as cancer tissue samples. All samples were collected fresh from theatre and samples processed within 1 hr of surgical excision. Fresh samples were frozen rapidly in Liquid Nitrogen and stored at −80° C. Ethical approval was obtained from the NRES Committee East of England (reference number 15/EE/0192).

The second set of samples were obtained from the clinical trial “The Effect of a Progesterone Receptor Modulator on Breast Tissue in Women With BRCA-1 and -2 Mutations—a Placebo Controlled RCT” (ClinicalTrials.gov Identifier: NCT01898312; regional ethical review board at Karolinska Institutet permit 2009/144-31/4). Study subjects were healthy premenopausal women aged 18-43 years with regular menstrual cycles lasting 25-35 days and with no contraindications to mifepristone. The main exclusion criteria were: use of any hormonal or intrauterine contraception and pregnancy or breastfeeding 2 months prior to the study; a history of breast cancer or other malignancies and adnexal abnormality upon transvaginal ultrasound examination. All women were instructed to use barrier contraceptive methods throughout the duration of the study.

After signing an informed consent, study subjects were randomised into two groups. One group (i.e. 11 BRCA carriers and 9 controls) was treated with 50 mg mifepristone (one quarter of 200 mg Mifegyne®, Exelgyn, Paris, France) every other day for two months (56 days) starting on the first day of the menstrual cycle. As mifepristone is only available in 200 mg tablets in Sweden, a study nurse divided the tablets into 4 parts and instructed the study subjects to take one part every other day. The placebo group (i.e. 4 BRCA carriers and 11 controls) received B-vitamin tablets which are visually identical (one quarter of TrioBe® Recip) to mifepristone. Tablets were dispensed for two weeks at a time.

Core needle aspiration biopsies were collected at baseline, before treatment, and at the end of treatment during the luteal phase. The biopsies were collected under ultrasound guidance from the upper outer quadrant of one breast using a 14 Gauge needle with an outer diameter of 2.2 mm. The end-of-treatment breast biopsy was taken from the same area.

### Sample Processing and DNA Extraction

When preparing for sample storage in the laboratory, cervical smear samples were poured into 50 ml Falcon tubes and left to sediment at room temperature for 2 hours. 1 mL wide bore tips were then used to transfer the enriched cellular sediment into a 2 mL vial. The cervical sediments were washed twice with PBS, lysed, and stored temporarily at −20° C. ahead of extraction. The Copan 4N6FLOQ Buccal Swabs were cut and lysed sequentially in the same aliquot of lysis buffer prior to temporary storage at −20° C. ahead of extraction. Whole blood samples were simply held transiently at −20° C. until DNA extraction. DNA was extracted from whole blood, cervical and buccal tissue lysates on a Hamilton Star liquid handling platform using the Nucleo-Mag Blood 200 ul kit (Macherey Nagel, cat #744501.4) with prior modifications for optimal lysis of cervical cell pellets and paired buccal swabs. For breast tissues, DNA was extracted from up to 40 mg of tissue using the Lipid Tissue kit from Macherey Nagel (cat #740471.50), and the manufacturer's instructions were followed. DNA concentration and quality absorbance ratios were measured using Nanodrop-8000, Thermoscientific Inc. Extracted DNA was stored at −80° C. until further analysis.

### DNA Methylation Array Analysis

Cervical, buccal and breast tissue DNA was normalised to 25 ng/ul and 500 ng total DNA was bisulfite modified using the EZ-96 DNA Methylation-Lightning kit (Zymo Research Corp, cat #D5047) on the Hamilton Star Liquid handling platform. 8 ul of modified DNA was subjected to methylation analysis on the Illumina InfiniumMethylation EPIC BeadChip (Illumina, Calif., USA) at UCL Genomics according to the manufacturer's standard protocol.

### Methylation Analysis

Methylation microarrays were processed using the R package minfi. Any samples with median methylated and unmethylated intensities <9.5 were removed. The champ.filter function in the R package ChAMP was used to filter non-CpG (2,932), SNP-related (81,531), and multi-hit (49) probes. Any probes with a detection p-value >0.01 in more than 10% of samples were removed. The beta mixture quantile normalisation (BMIQ) algorithm was used to normalise beta values (via the champ.norm function). Since BMIQ does not allow for missing values, the champ.impute function was therefore used to impute any missing values (0.008% of values were missing).

In the discovery cohort, 144 samples and 2,554 probes were removed during QC (one plate containing 96 samples was removed due to low quality) resulting in a final data matrix with 1,063 samples and 779,773 CpGs. The external validation dataset consisted of 335 samples and 781,570 probes after 17 samples and 2,868 probes were removed during QC. No samples and 2,556 probes were removed from the buccal dataset and the resulting data matrix comprised 404 samples and 780,049 CpGs.

The fraction of immune cell contamination, and the relative proportions of different immune cell subtypes in each sample, were estimated using the EpiDISH algorithm using the epithelial, fibroblast and immune cell reference dataset. The top 1,000 most variable probes (ranked by standard deviation) were used in a principal component analysis. Statistical tests were performed in order to identify any anomalous associations between plate, sentrix position, date of array processing, date of DNA creation, study centre, immune contamination fraction, age, type (case versus control) and the top ten principal components. In the discovery cohort, one plate (containing 96 samples) was found to have anomalous beta values and was removed from the dataset. Finally, two-thirds of the discovery dataset was randomly selected for use as the training dataset and the remaining third was allocated to the internal validation dataset. This split was carried out once, and the same training and validation sets were used in all subsequent analyses.

113 samples were downloaded from the ENCODE database (https://www.encodeproject.org/; see Table 3). The beta mixture quantile normalisation was applied to these samples after using minfi to extract beta values. The WID-BC-index was then computed using the 40,753 required CpGs.

TABLE 3
Overview of the ENCODE samples used.

| | Biosample term | Biosample | |
|---|---|---|---|
| Experiment | name | type | Type |
| ENCSR001NC | thyroid gland | Tissue | epithelial |
| ENCSR002EIR | sigmoid colon | Tissue | epithelial |
| ENCSR002LED | transverse colon | Tissue | epithelial |
| ENCSR039CG | tibial nerve | Tissue | non_epitheli |
| ENCSR050XGE | tibial artery | Tissue | non_epitheli |
| ENCSR061NRX | tibial nerve | Tissue | non_epitheli |
| ENCSR069UIN | gastrocnemius | Tissue | non_epitheli |
| | medialis | | |
| ENCSR079OX | bipolar neuron | in vitro | non_epitheli |
| | | differentiated | |
| ENCSR080HYX | prostate gland | Tissue | epithelial |
| ENCSR090CRZ | transverse colon | Tissue | epithelial |
| ENCSR096DB | stomach | Tissue | epithelial |
| ENCSR097SQ | esophagus squamous | Tissue | epithelial |
| ENCSR113TRL | upper lobe of left | Tissue | epithelial |
| | lung | | |
| ENCSR147FPX | sigmoid colon | Tissue | epithelial |
| ENCSR148KKY | mammary epithelial | primary cell | epithelial |
| | cell | | |
| ENCSR154ELD | esophagus squamous | Tissue | epithelial |
| ENCSR173NTZ | thyroid gland | Tissue | epithelial |
| ENCSR190PQ | heart left ventricle | Tissue | non_epitheli |
| ENCSR190WY | vagina | Tissue | epithelial |
| ENCSR193BIR | gastrocnemius | Tissue | non_epitheli |
| | medialis | | |
| ENCSR200LAH | esophagus squamous | Tissue | epithelial |
| ENCSR201NNA | Peyer's patch | Tissue | non_epitheli |
| ENCSR203HAK | lower leg skin | Tissue | epithelial |
| ENCSR209XGZ | adrenal gland | Tissue | non_epitheli |
| ENCSR215SBD | gastroesophageal | Tissue | non_epitheli |
| | sphincter | | |
| ENCSR244HUE | kidney epithelial | primary cell | epithelial |
| | cell | | |
| ENCSR246VHI | neural progenitor | in vitro | non_epitheli |
| | cell | differentiated | |
| ENCSR248EIV | gastrocnemius | Tissue | non_epitheli |
| | medialis | | |
| ENCSR262IUB | gastroesophageal | Tissue | non_epitheli |
| | sphincter | | |
| ENCSR276YFP | spleen | Tissue | non_epitheli |
| ENCSR280LMY | right atrium | Tissue | non_epitheli |
| | auricular region | | |
| ENCSR301SLO | lower leg skin | Tissue | epithelial |
| ENCSR304AIL | testis | Tissue | epithelial |
| ENCSR306JCS | omental fat pad | Tissue | non_epitheli |
| ENCSR312XVJ | esophagus squamous | Tissue | epithelial |
| ENCSR315CV | subcutaneous | in vitro | non_epitheli |
| | adipose tissue | differentiated | |
| ENCSR329WA | thyroid gland | Tissue | epithelial |
| ENCSR340GP | stomach | Tissue | epithelial |
| ENCSR343SAU | skeletal muscle | primary cell | non_epitheli |
| | myoblast | | |
| ENCSR353IUV | suprapubic skin | Tissue | epithelial |
| ENCSR371REA | adrenal gland | Tissue | non_epitheli |
| ENCSR392LYN | breast epithelium | Tissue | epithelial |
| ENCSR393CCK | breast epithelium | in vitro | epithelial |
| | | differentiated | |
| ENCSR394PUR | vagina | Tissue | epithelial |
| ENCSR399KXO | adrenal gland | Tissue | non_epitheli |
| ENCSR406QEF | thyroid gland | Tissue | epithelial |
| ENCSR415PYP | prostate gland | Tissue | epithelial |
| ENCSR418YFM | subcutaneous | Tissue | non_epitheli |
| | adipose tissue | | |
| ENCSR420WU | smooth muscle cell | in vitro | non_epitheli |
| | | differentiated | |
| ENCSR422EPB | epithelial cell of | primary cell | epithelial |
| | alveolus of lung | | |
| ENCSR425TKT | tibial artery | Tissue | non_epitheli |
| ENCSR426CDE | upper lobe of left | Tissue | epithelial |
| | lung | | |
| ENCSR444YPR | upper lobe of left | Tissue | epithelial |
| | lung | | |
| ENCSR448FCV | suprapubic skin | Tissue | epithelial |
| ENCSR449VM | cardiac muscle cell | Tissue | non_epitheli |
| ENCSR461NFO | lower leg skin | Tissue | epithelial |
| ENCSR467AVQ | Peyer's patch | Tissue | non_epitheli |
| ENCSR468IFF | myotube | in vitro | non_epitheli |
| | | differentiated | |
| ENCSR472PKR | esophagus | Tissue | epithelial |
| | muscularis mucosa | | |
| ENCSR486SM | ascending aorta | Tissue | non_epitheli |
| ENCSR493EGV | upper lobe of left | Tissue | epithelial |
| | lung | | |
| ENCSR511SNB | ovary | Tissue | epithelial |
| ENCSR515ZCU | heart left ventricle | Tissue | non_epitheli |
| ENCSR517JQA | right atrium | Tissue | non_epitheli |
| | auricular region | | |
| ENCSR528NFI | non-pigmented | primary cell | epithelial |
| | ciliary epithelial | | |
| ENCSR551DKY | tibial nerve | Tissue | non_epitheli |
| ENCSR558ACF | transverse colon | Tissue | epithelial |
| ENCSR575WO | suprapubic skin | Tissue | epithelial |
| ENCSR580LHO | transverse colon | Tissue | epithelial |
| ENCSR582BM | coronary artery | Tissue | non_epitheli |
| ENCSR583ILE | mammary epithelial | primary cell | epithelial |
| | cell | | |
| ENCSR584HJL | spleen | Tissue | non_epitheli |
| ENCSR597BUD | body of pancreas | Tissue | epithelial |
| ENCSR598BUX | gastroesophageal | Tissue | non_epitheli |
| | sphincter | | |
| ENCSR604PTS | lower leg skin | Tissue | epithelial |
| ENCSR646XKN | tibial artery | Tissue | non_epitheli |
| ENCSR662NBA | omental fat pad | Tissue | non_epitheli |
| ENCSR675IVH | epithelial cell of | primary cell | epithelial |
| | proximal tubule | | |
| ENCSR688OH | coronary artery | Tissue | non_epitheli |
| ENCSR701SVQ | esophagus | Tissue | epithelial |
| | muscularis mucosa | | |
| ENCSR705PDD | body of pancreas | Tissue | epithelial |
| ENCSR719GFJ | Peyer's patch | Tissue | non_epitheli |
| ENCSR729VBL | tibial nerve | Tissue | non_epitheli |
| ENCSR731SPT | ascending aorta | Tissue | non_epitheli |
| ENCSR733HHJ | subcutaneous | Tissue | non_epitheli |
| | tadipose issue | | |
| ENCSR733WX | omental fat pad | Tissue | non_epitheli |
| ENCSR738XQ | skeletal muscle | primary cell | non_epitheli |
| | myoblast | | |
| ENCSR744AJV | ovary | Tissue | epithelial |
| ENCSR754ANZ | iris pigment | primary cell | epithelial |
| | epithelial cell | | |
| ENCSR756BTI | spleen | Tissue | non_epitheli |
| ENCSR773EP | sigmoid colon | Tissue | epithelial |
| ENCSR792ATG | suprapubic skin | Tissue | epithelial |
| ENCSR803DDS | uterus | Tissue | epithelial |
| ENCSR809OPY | myotube | primary cell | non_epitheli |
| ENCSR822VTU | esophagus | Tissue | epithelial |
| | muscularis mucosa | | |
| ENCSR827WS | sigmoid colon | Tissue | epithelial |
| ENCSR846DD | breast epithelium | Tissue | epithelial |
| ENCSR847BAX | astrocyte | primary cell | non_epitheli |
| ENCSR871SFO | esophagus | Tissue | epithelial |
| | muscularis mucosa | | |
| ENCSR889TZA | uterus | Tissue | epithelial |
| ENCSR899LHQ | retinal pigment | primary cell | epithelial |
| | epithelial cell | | |
| ENCSR899UFG | stomach | Tissue | epithelial |
| ENCSR905RZU | gastroesophageal | Tissue | non_epitheli |
| | sphincter | | |
| ENCSR922EBK | body of pancreas | Tissue | epithelial |
| ENCSR937LYZ | right lobe of liver | Tissue | epithelial |
| ENCSR940ZHS | body of pancreas | Tissue | epithelial |
| ENCSR942OLI | testis | Tissue | epithelial |
| ENCSR955LKF | hepatocyte | primary cell | epithelial |
| ENCSR962JMK | subcutaneous | Tissue | non_epitheli |
| | adipose tissue | | |
| ENCSR963NN | renal cortical | primary cell | epithelial |
| | epithelial cell | | |
| ENCSR976HY | choroid plexus | Tissue | non_epitheli |
| | epithelial cell | | |
| ENCSR991SII | tibial artery | Tissue | non_epitheli |
| ENCSR995PG | omental fat pad | Tissue | non_epitheli |

### Statistical Analyses for Classifier Development

Contamination by immune cells presented a challenge with respect to the identification of differentially methylated positions (DMPs) as differential methylation that occurred solely in epithelial cells was diminished in samples with high IC and vice versa. In order to overcome this, we linearly regressed the beta values on IC for each CpG site, the linear models being fitted to cases and controls separately. The intercept points at IC=0 were used as estimates of mean beta values in cases and controls in a pure epithelial cell population. The difference between these intercept points provided a delta-beta estimate in epithelial cells. The difference between intercept points at IC=1 provided immune cell delta-beta estimates. Two lists of ranked CpGs were produced according to delta-beta estimates in epithelial and immune cells.

The R package glmnet was used to train classifiers with a mixing parameter value of alpha=0 (ridge penalty) and alpha=1 (lasso penalty) with binomial response type. Data from the training dataset were used to fit the classifiers. Ten-fold cross-validation was used internally by the cv.glmnet function in order to determine the optimal value of the regularisation parameter lambda. The AUC was used as a metric of classifier performance which was evaluated on the internal validation dataset as a function of n, the number of CpGs used as inputs during training. For individual i, denote beta values from the top n CpGs ranked by epithelial and immune delta-betas as xᵢ₁, . . . , xᵢₙ and yᵢ₁, . . . , yᵢₙ respectively. Denote the IC fraction as ρᵢ. The following terms were used as inputs to the ridge and lasso classifiers:
xᵢ₁, . . . , xᵢₙ, (1−ρᵢ)xᵢ₁, . . . , (1−ρᵢ)xᵢₙ, yᵢ₁, . . . , yᵢₙ, ρᵢyᵢ₁, . . . , ρᵢyᵢₙ
   Note that the classifier is mathematically equivalent to the index described above. In addition to a classifier with these interaction terms, we also trained a second type of classifier based on xᵢ₁, . . . , xᵢₙ alone.

The optimal classifier was selected based on the highest AUC obtained in the internal validation dataset. Once the optimal number of inputs was determined, the training and internal validation datasets were combined and the classifier was refitted using the entire discovery dataset with alpha and lambda fixed to their optimal values. This finalised classifier was then applied to the external validation dataset and the corresponding AUC was computed.

### Enrichment Analysis

The epithelial delta-beta estimates were used to compute the top 1,000 hyper and hypo CpGs. These were used as inputs to the eFORGE 2.0 tool²⁰ (accessed at https://eforge.altiusinstitute.org/). Data from the “Consolidated Roadmap Epigenomics DHS” were used for the analysis. The default options of 1 kb proximity window, 1,000 background repetitions, and strict and marginal significance thresholds of 0.01 and 0.05 were used.

A gene set enrichment analysis (GSEA)²¹ was carried out by first selecting for each gene TSS200 region the CpG with the largest epithelial delta-beta estimate (both hyper- and hypo-methylated). Genes were then ranked according to the absolute value of these delta-beta estimates. The C2 curated gene set, c2.all.v6.2.symbols.gmt, was downloaded from MSigDB. The fgsea R package was used to perform the enrichment analysis with parameters minSize, maxSize, and nperm set to 15, 500, and 10,000 respectively.

### Analysis of Breast Tissue Samples

The cell type composition of each sample was estimated using EpIDISH with the epithelial, fibroblast, fat, and immune cell reference dataset. In contrast to cervical samples, fat cells constituted a substantial proportion of each sample. Results from cervical smear data indicated that the index performs independently of epithelial and immune proportions (fibroblasts formed a negligible proportion of cells). A linear adjustment was therefore made for fat content by splitting the samples into normal, BRCA carrier, adjacent, and TNBC groups. We linearly regressed the WID-BC-index on fat in each group and obtained an estimate of what the index values would be if all four groups had the same fat composition. Similarly, samples from the mifepristone trial were split into mifepristone before, mifepristone after, placebo before, and placebo after groups. Within each group we linearly regressed the index on fat proportion in order to obtain estimates of the index after adjustment for fat content.

### SNP Genotyping, QC and Imputation

In total, 318 breast cancer case subjects and 850 controls from the methylation discovery cohort were taken forward for genotyping using an Illumina 650k Infinium Global Screening Array (GSA). Whole blood DNA was normalised to 75 ng/ul and a total of 300 ng applied to the Infinium Global Screening Array—24 V2 (Illumina, Calif., USA) at UCL Genomics according to the manufacturer's standard protocol.

One control subject from this cohort failed to genotype. Genotype calling was performed using GenomeStudio, with genetic variants found to be clustering poorly being removed from further analyses. For duplicate genetic variant pairs, the variant within each pair with the lowest calling and clustering score was excluded. Autosomal SNPs were used in subsequent QC and PRS analyses (except for checks for sex mismatches, where the X chromosome was used to infer sex).

General subject and single nucleotide polymorphism (SNP) quality control (QC) was performed using PLINK version 1.9²⁷. Three breast cancer cases and eight controls with a call rate less than 95% were excluded. One breast cancer case and three controls were further removed due to genetically inferred sex not being female. Genetic variants with a missing genotype rate greater than 5%, minor allele frequency (MAF) less than 1% or a significant departure from Hardy-Weinberg equilibrium (p-value <5×10-6) were excluded.

KING′, a relatedness inference algorithm, was used to identify duplicate/monozygotic twin or first-degree relative pairs. One control subject pair was identified as being a duplicate/monozygotic twin pair, and nine control pairs were inferred to be first-degree relatives. The subject within each related pair with the lowest call rate was excluded. After performing QC, 314 breast cancer case subjects, 816 controls and 479,105 variants were retained in the SNP discovery sample.

Non-European subjects were identified by plotting the top two principal components, generated using GCTA version 1.26.0, for the SNP discovery samples and 270 HapMap phase II release 23 samples (CEU, YRI, JPT and CHB individuals) downloaded in PLINK-formatted binary files. Subjects found not to cluster around HapMap European samples were excluded from further analyses. After excluding non-European subjects, 305 breast cancer cases and 754 controls were retained in the SNP discovery sample.

Using the Michigan Imputation Server²⁹ and 1000 Genomes Phase 3 reference panel, the SNP discovery dataset went through further QC before being phased (Eagle2) and imputed. Variants where strand, allele, genetic position or allele frequencies were not concordant with the 1000 Genomes Phase 3 reference panel were removed before phasing and imputation using Strand Tools.

After imputation, exclusion of variants with imputation R²<0.5 and removal of variants observed to have 3 or more alleles, 303 of the 313 SNPs used by Mavaddat et al.²² to develop a 313 SNP breast cancer polygenic risk score (PRS) were successfully imputed. We constructed a breast cancer PRS for each subject in the discovery cohort, such that the PRS is equal to:

$$P R {S}_{j} = {\sum }_{i = 1}^{303} \hat{{β}_{ι}} {x}_{i j}$$

where, \hat{β}ₗ is the log odds ratio for the i-th SNP taken from publically available Oncoarray summary association results' (combined Oncoarray, iCOGs and BCAC overall breast cancer beta values) and x₁ⱼ is the number of copies of the effect allele present in each discovery cohort subject. Scores were generated using PLINK version 1.9.

### Statistical Analysis of Buccal Samples

Matched buccal samples were taken from a subset of 404 women in the discovery dataset. The WID-BC-index derived from the discovery dataset of cervical samples was computed in the buccal samples and the corresponding AUC was obtained. Of the buccal samples, 269 belonged to the training dataset and 135 to the internal validation dataset. A separate classifier was derived using the buccal samples alone and utilising the same protocol as described above. For the purposes of comparison, another classifier was developed using the 269 and 135 cervical samples that had matched buccal samples for training and validation respectively.

### Example 1

### Sample Heterogeneity and Differential Methylation

For the Discovery Set (FIG. 7), we collected samples from 285 women with primary breast cancers with poor prognosis features (defined by >2 cm cancers and/or lymph-node positive and/or grade 3 and/or hormone-receptor negative) from 14 European centres at the time of diagnosis and before treatment commenced, and 778 women without breast cancer (536 from the general population and 242 from women attending hospital for benign women-specific conditions) (FIG. 15). Epigenome-wide DNAme was analysed using an Illumina Infinium EPIC bead chip array which encompasses over 850,000 CpG sites¹⁸.

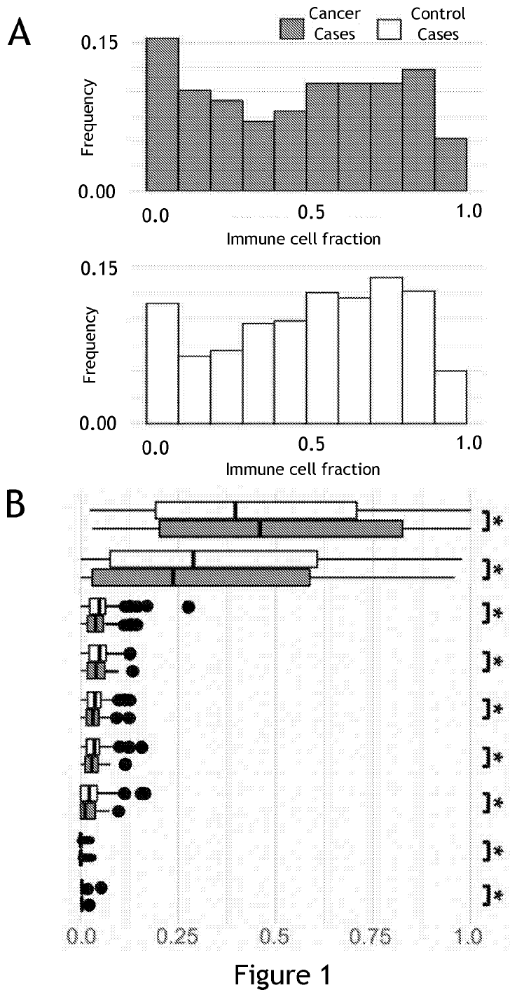

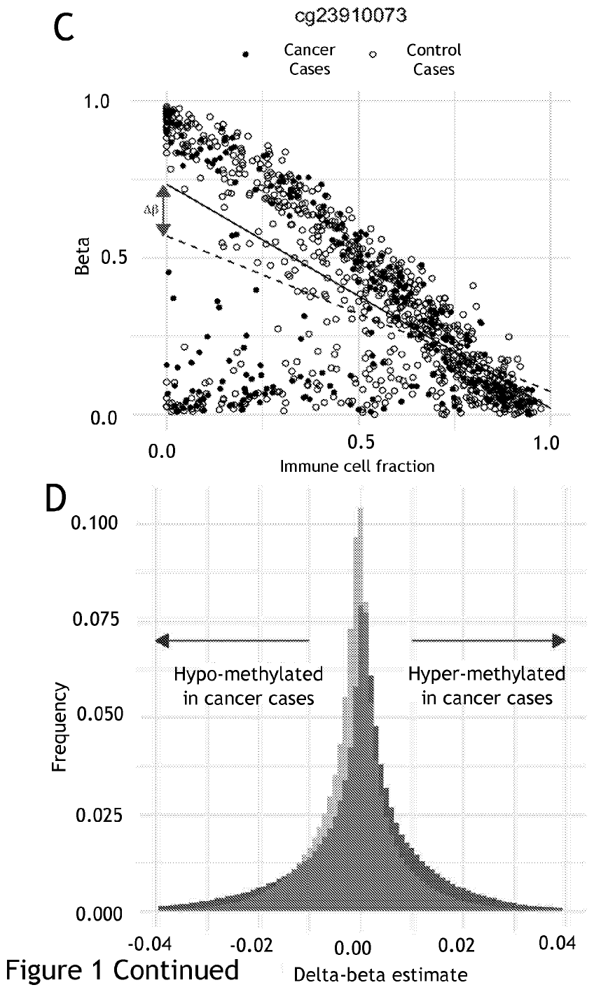

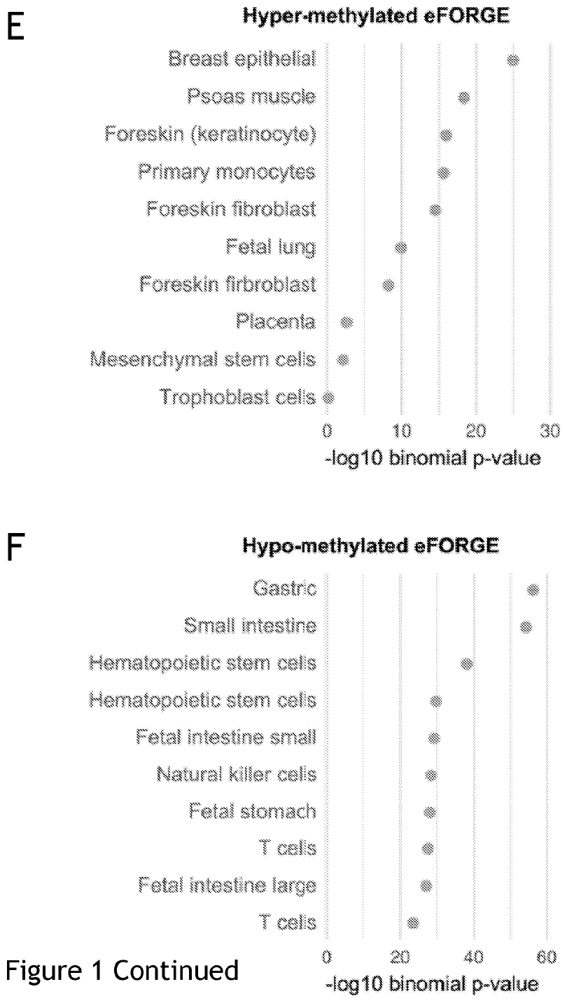

We assessed the level of cell type heterogeneity in each cervical smear sample using HEpiDISH¹⁹, an algorithm that infers the relative proportion of epithelial cells, fibroblasts, and seven subtypes of immune cells in each sample. The distribution of immune cell contamination (IC) was approximately uniform in both samples from cancer cases and controls. There was a significantly greater proportion of epithelial cells in cancers, and correspondingly fewer immune cells across all subtypes in the discovery dataset (FIGS. 1A & 1B, Wilcoxon signed rank tests, p<0.05). This difference was comparatively small however, and absent in the external validation dataset (FIG. 8).

Identifying CpGs with differential methylation between cases and controls was hampered by contaminating immune cells, since any differential methylation in epithelial cells was greatly diminished in samples with high IC (see example in FIG. 1C). In order to infer which CpGs may contain a potential discriminatory signal, we developed a statistical protocol to estimate the delta-beta (i.e. difference in mean proportion of methylated cells) between cases and controls in a pure epithelial cell sample, and a pure immune cell sample. We linearly regressed beta values on IC fraction in both cases and controls separately. The difference between the two points, where these lines intercept the y-axis at IC=0, gives an estimate of the delta-beta between cases and controls in pure epithelial cells. Conversely, the difference between intercept points at the IC=1 axis gives a delta-beta estimate in immune cells.

Larger delta-betas were observed in epithelial cells than immune cells (FIG. 1D). The eFORGE tool²⁰ was utilised in order to search for enrichment of cell-type specific CpGs in the top 1,000 hyper- and hypo-methylated epithelial CpGs. The strongest enrichment in (i) hyper-methylated CpGs was for breast epithelial cell-specific CpGs and muscle, fibroblasts and mesenchymal cells (FIG. 1E) and in (ii) hypo-methylated CpGs for a foetal-like program with enrichment for foetal large and small intestine, and stomach (FIG. 1F). These findings suggest that in cervical smear samples from breast cancer cases the epigenome has undergone an epigenetic re-programming which may be reflective of a limited capacity for mammary epithelial cell differentiation and a shift towards mesenchymal and foetal programs. In addition, a gene set enrichment analysis was performed using the Broad Institute's Molecular Signatures Database²¹ (FIG. 16) and breast cancer associated pathways were enriched in hypo-methylated genes.

### Example 2

### Development of Discriminatory Index

In order to derive a diagnostic methylation signature, termed the WID-BC-index, we used ridge and lasso regression to classify individuals as cases or controls. Classifiers were trained on two thirds of the discovery dataset (508 cancer-free controls, 190 breast cancer cases) and the remaining one third was used as an internal validation set (270 controls, 95 cases) with the intention of evaluating their performance as a function of the number of CpGs used to construct the index. The area under the receiver operator characteristic curve (AUC) was used as a measure of predictive performance.

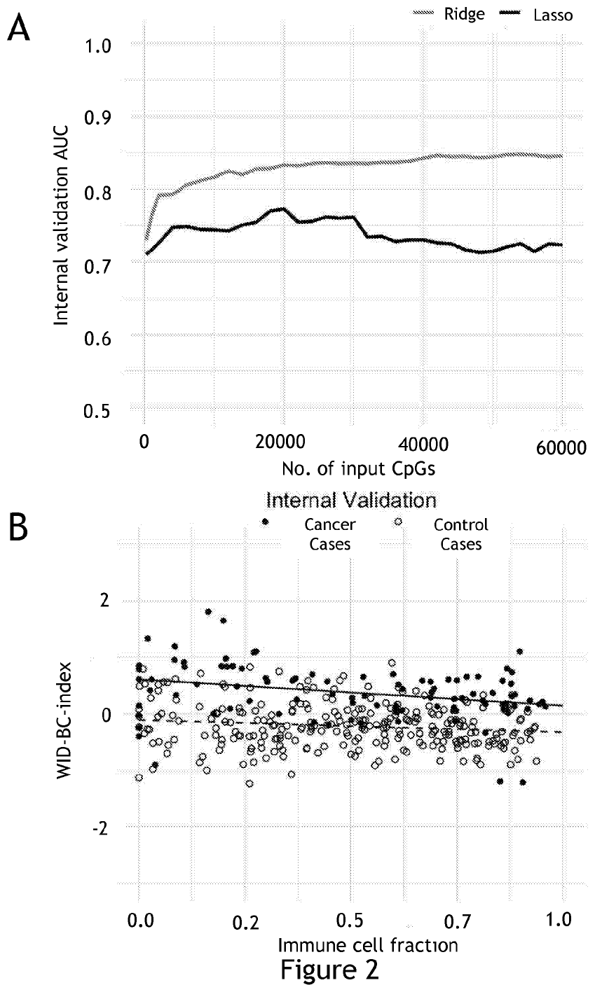

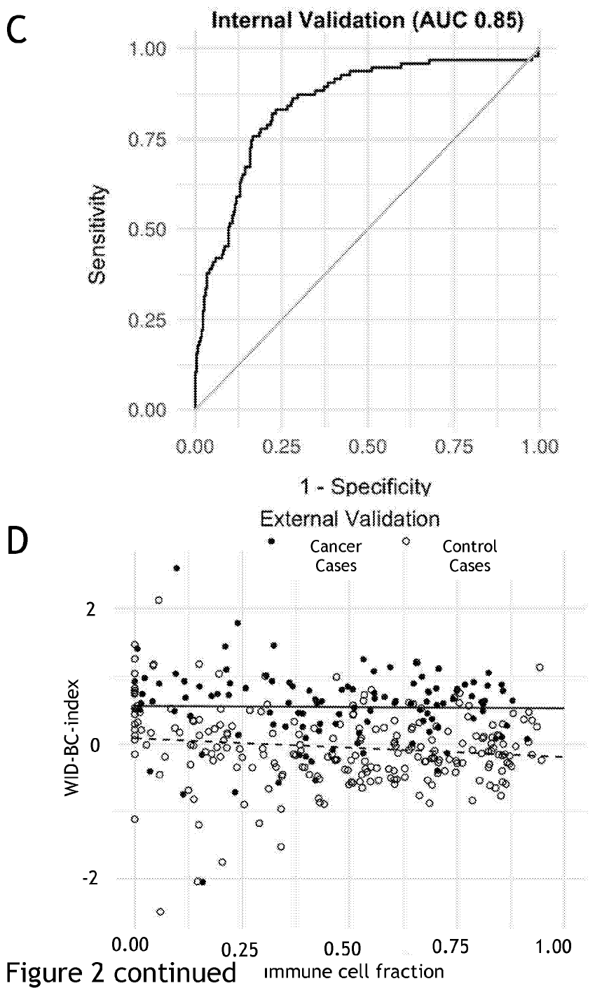

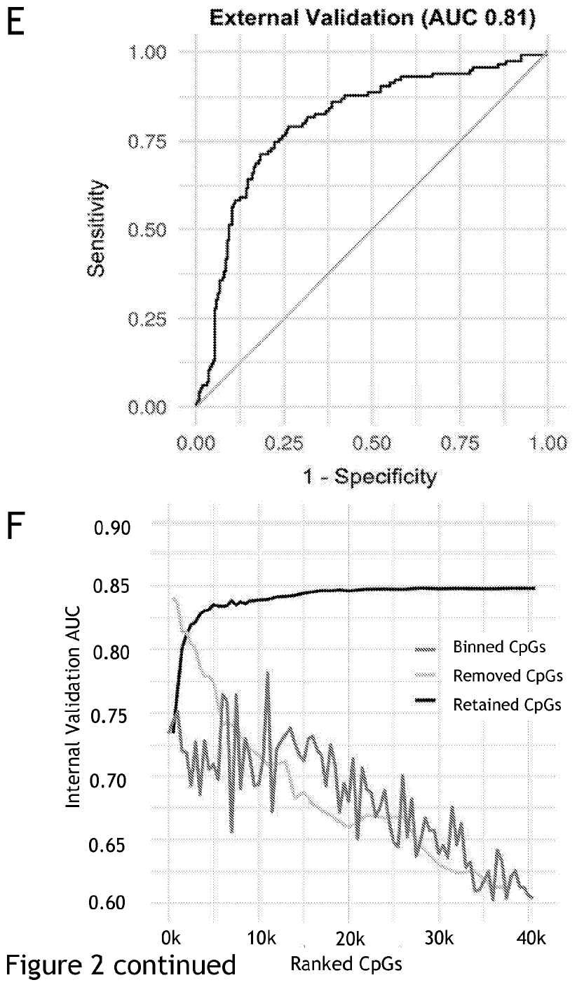

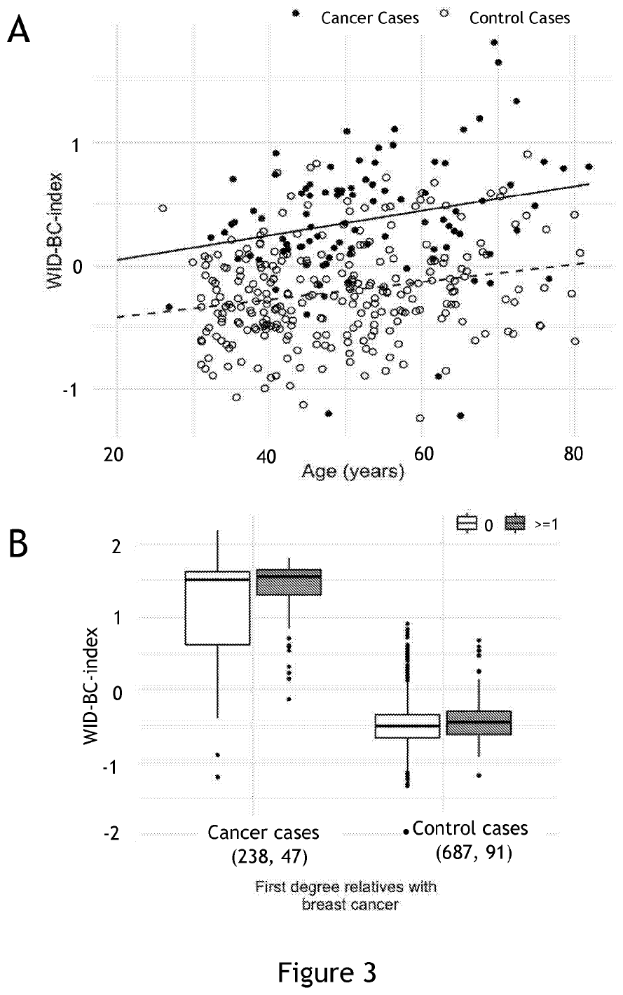

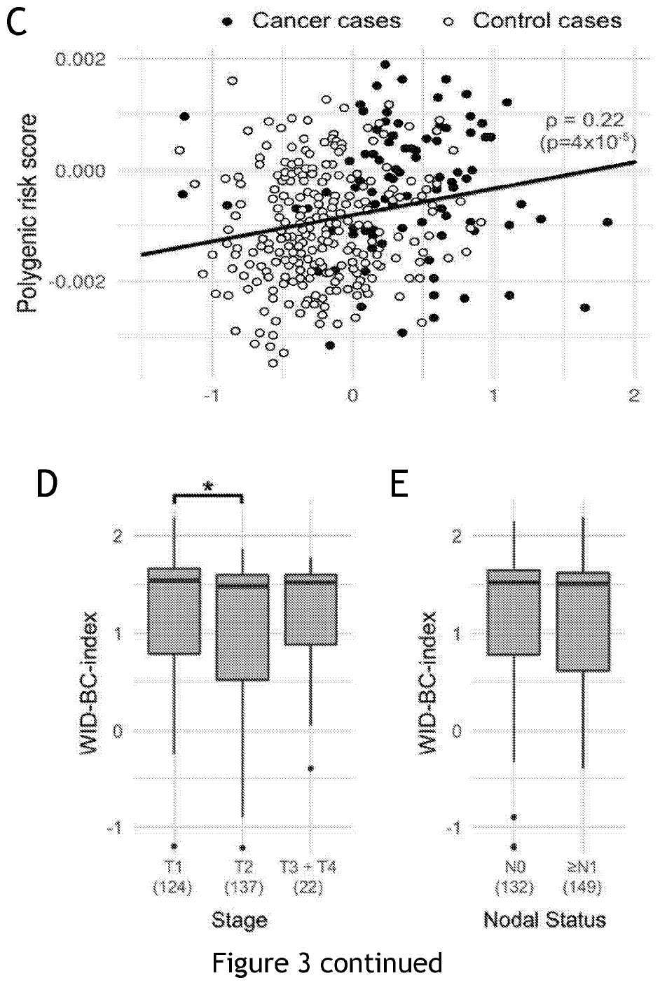

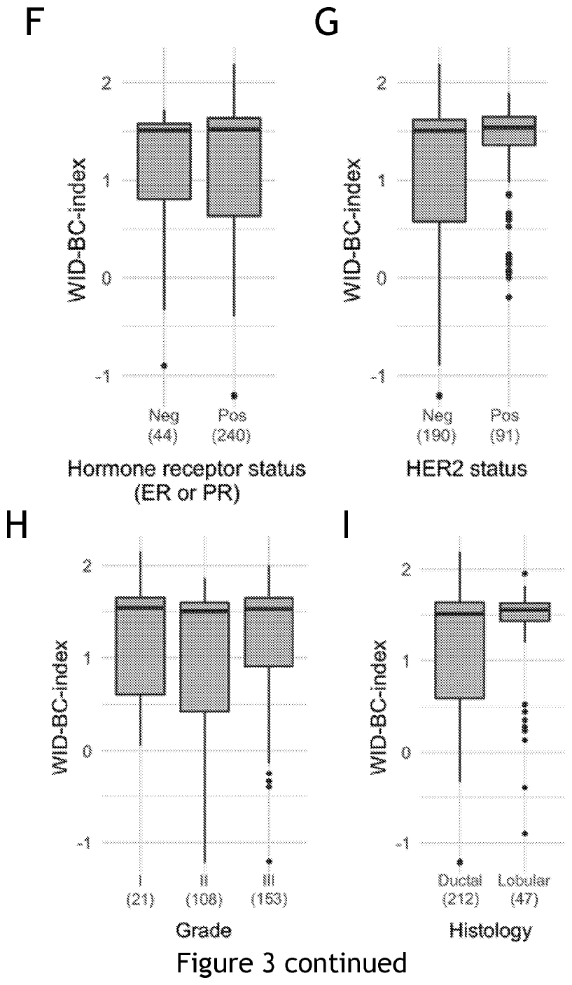

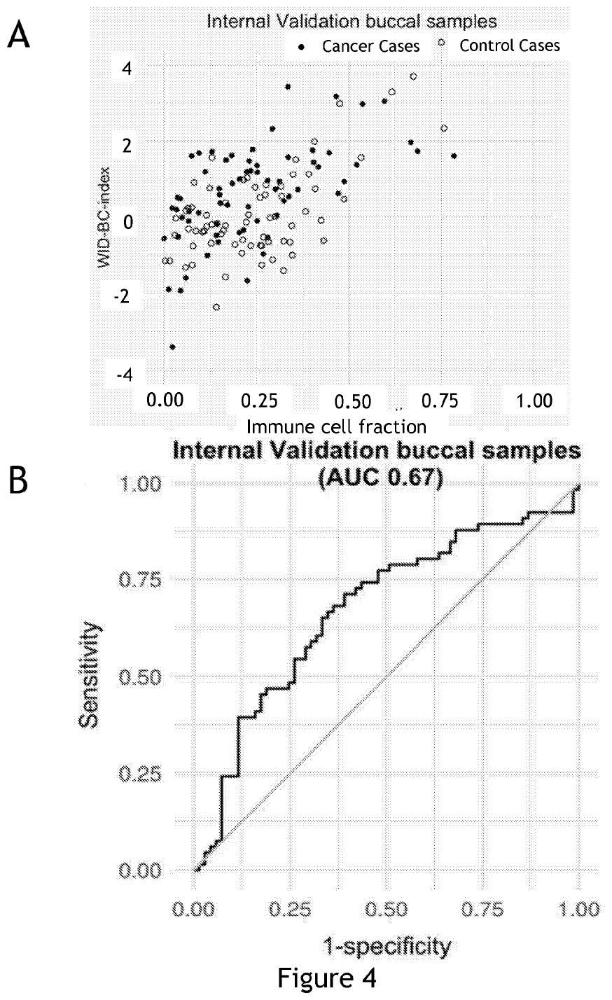

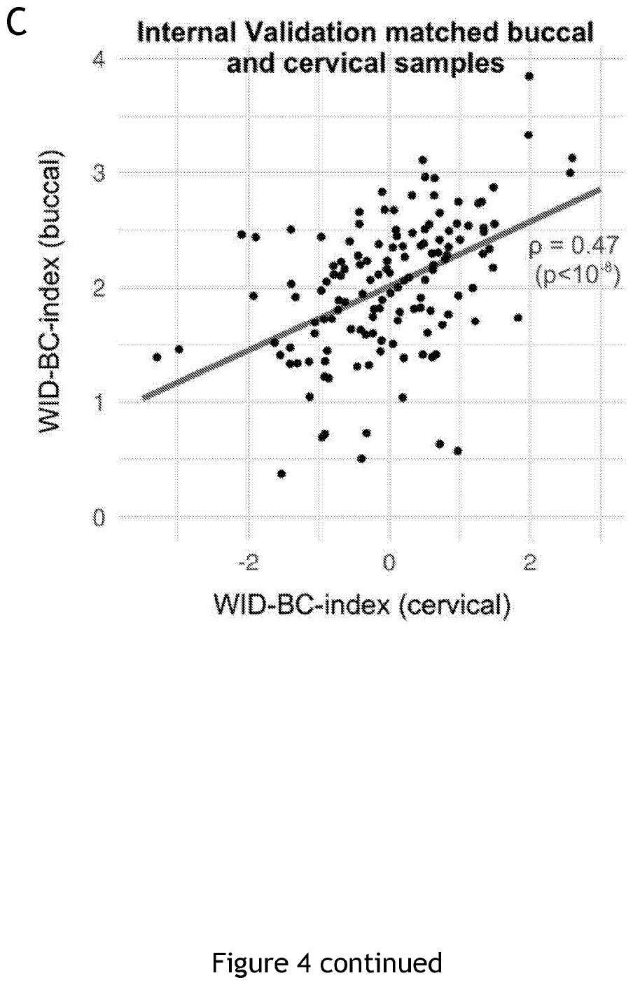

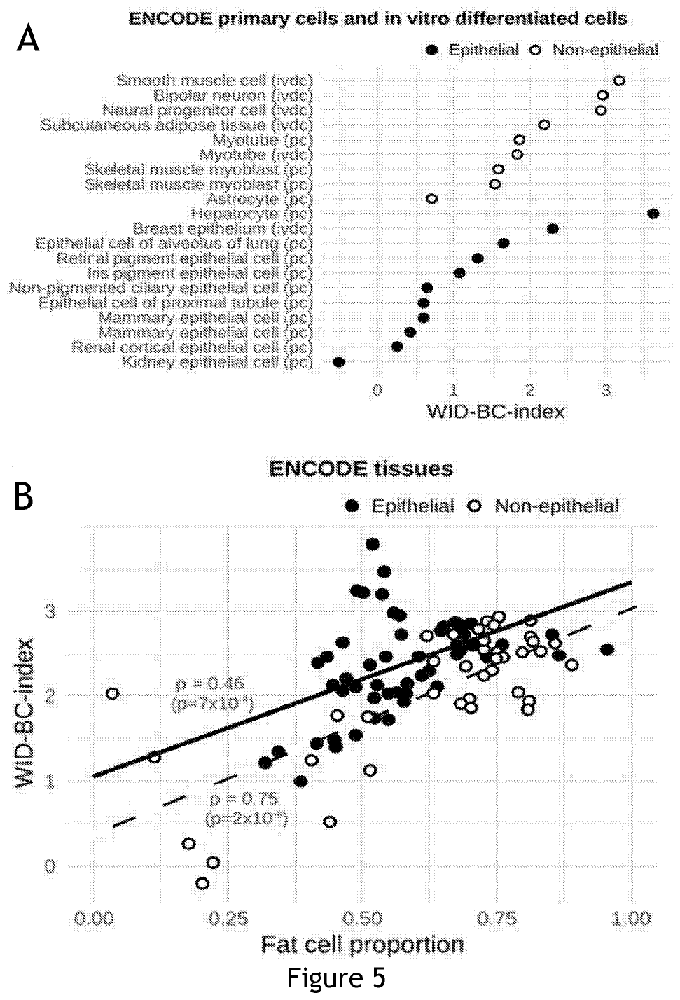

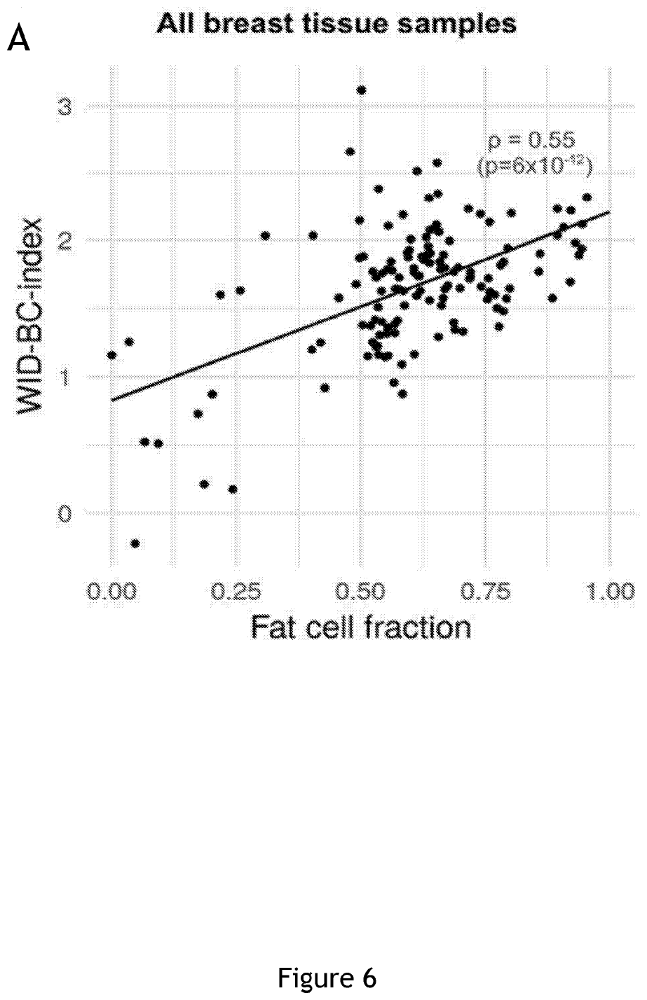

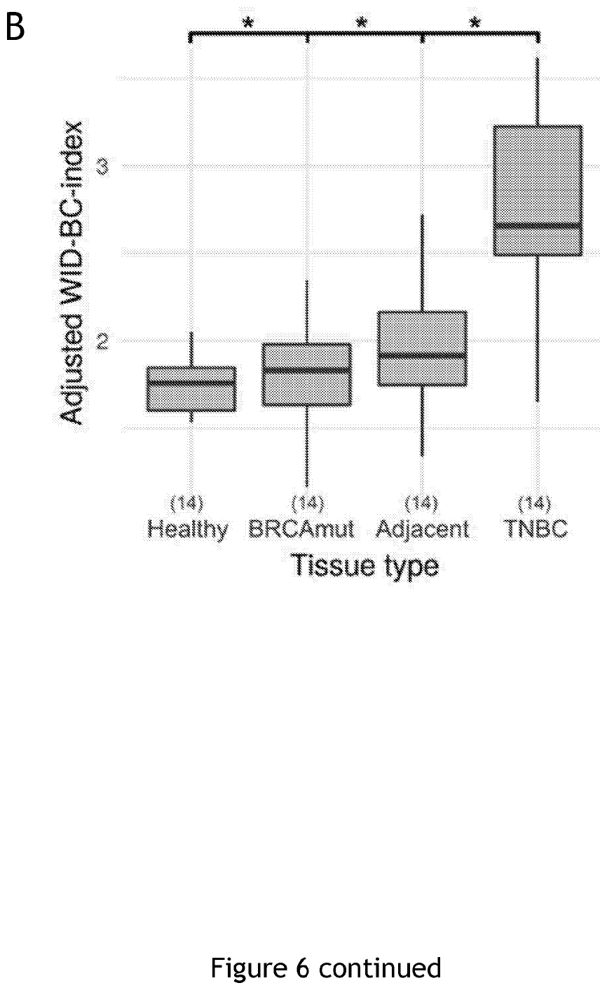

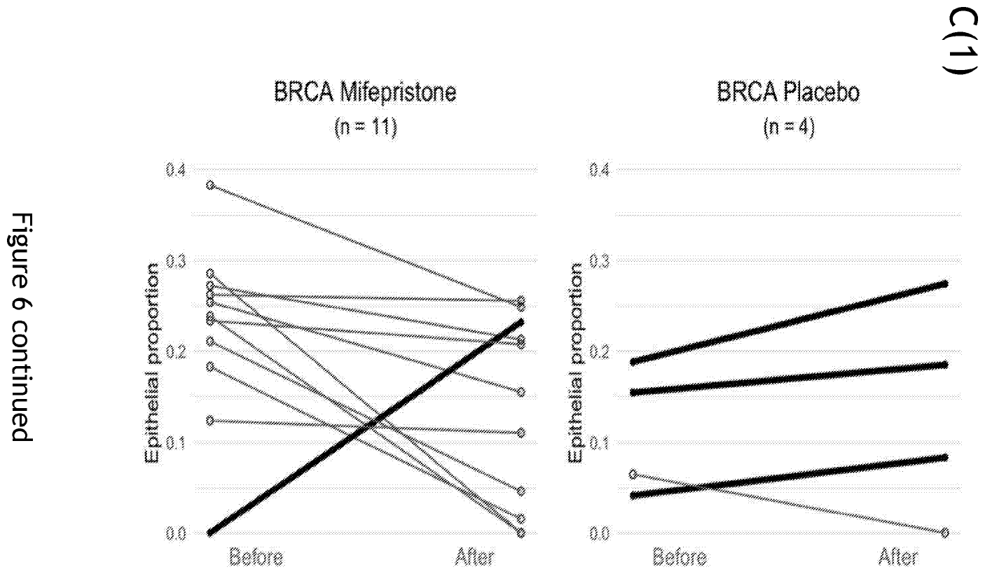

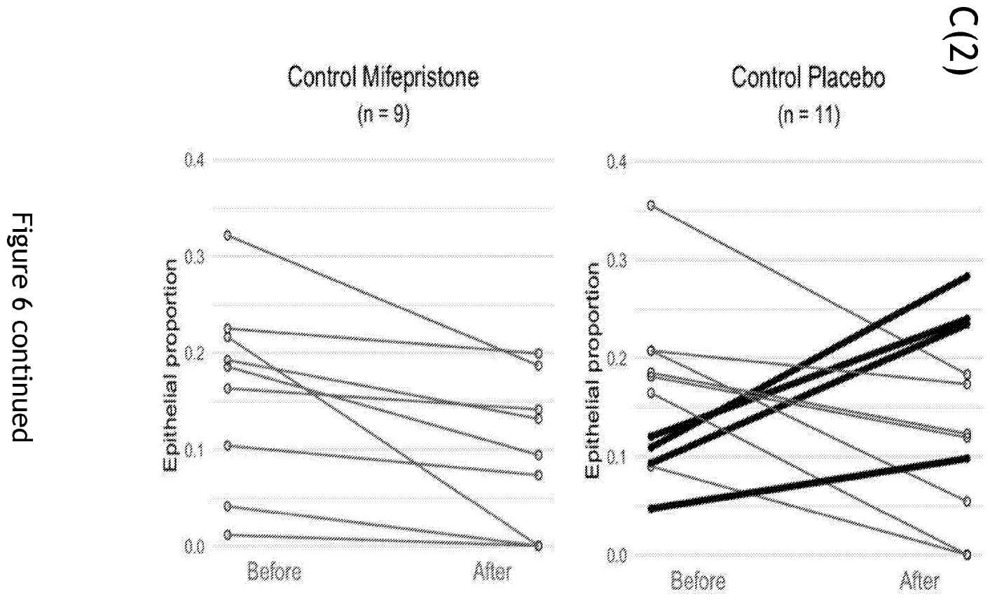

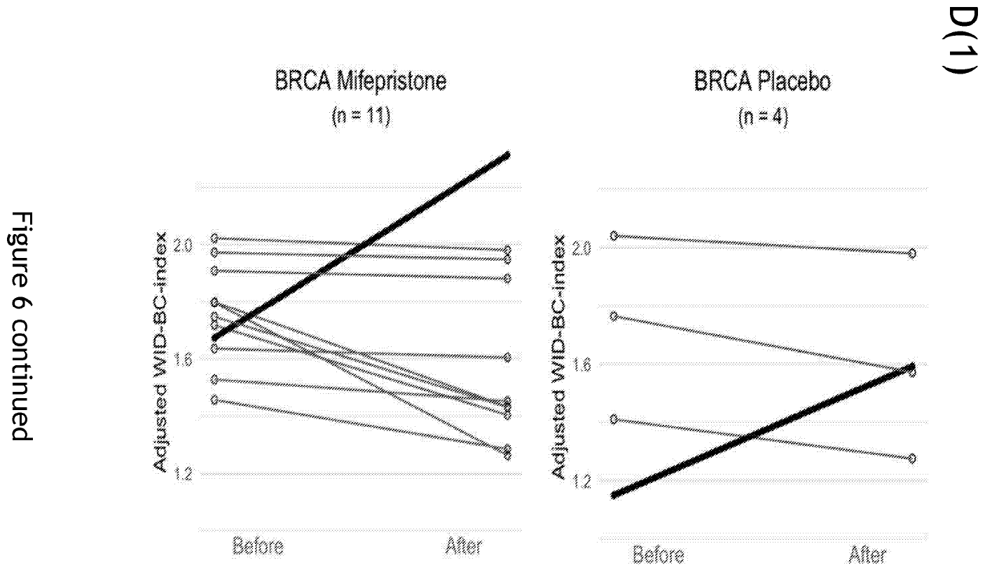

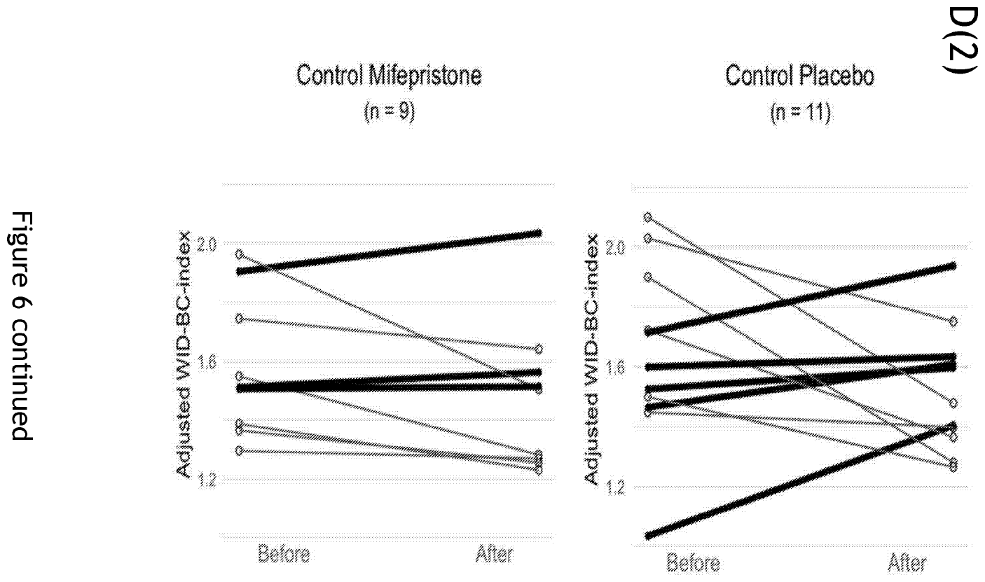

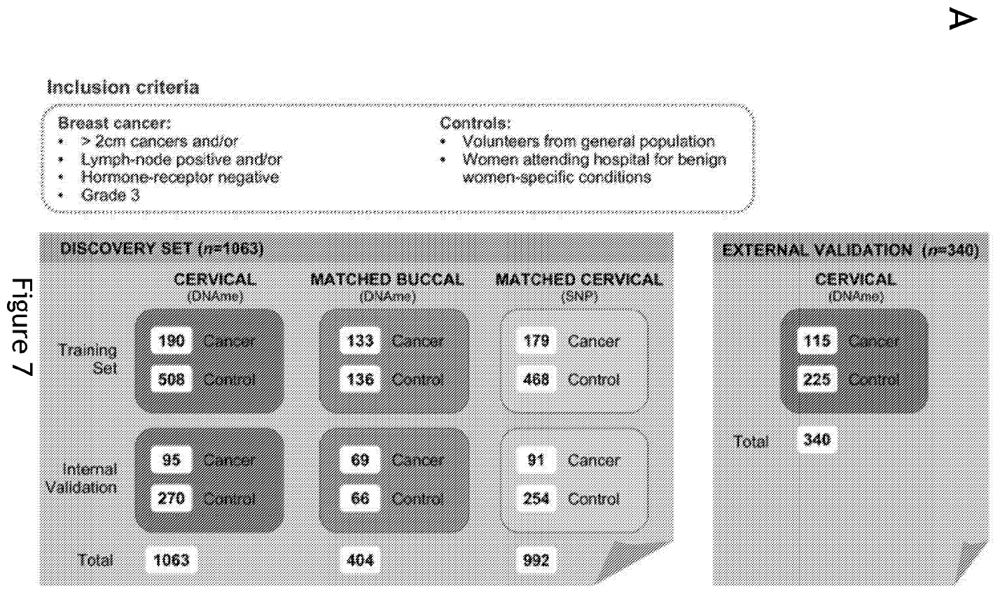

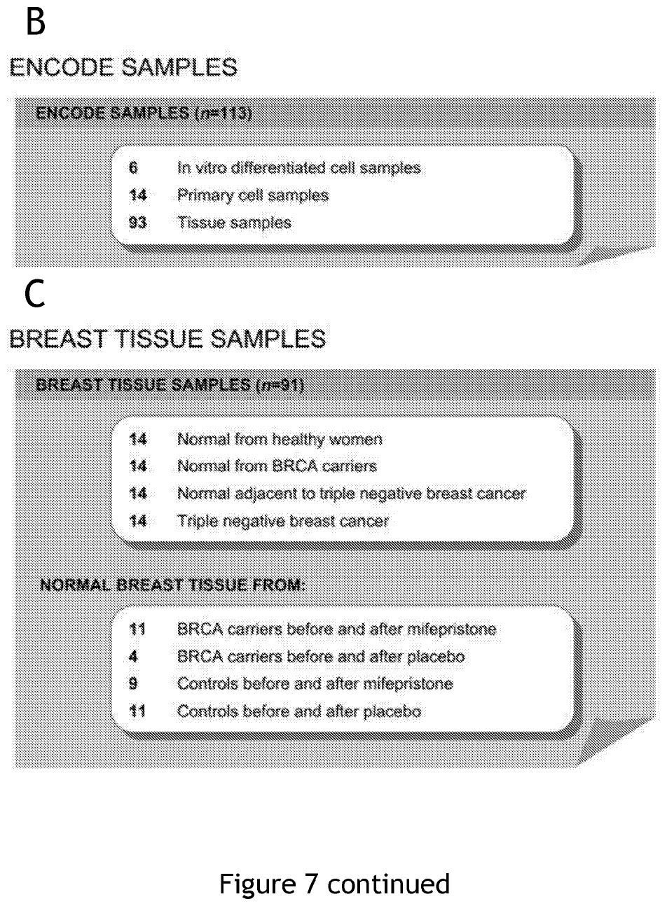

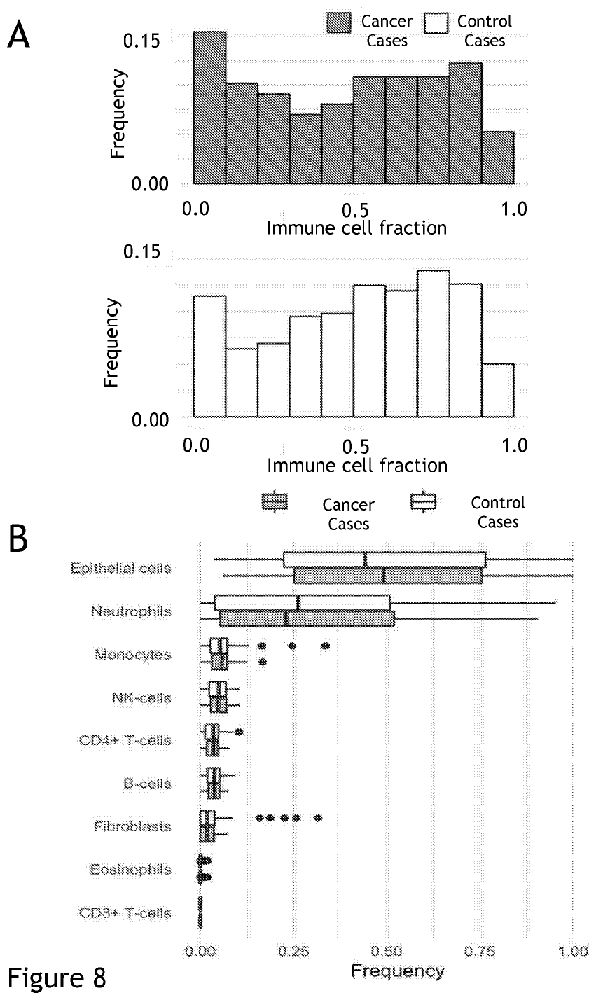

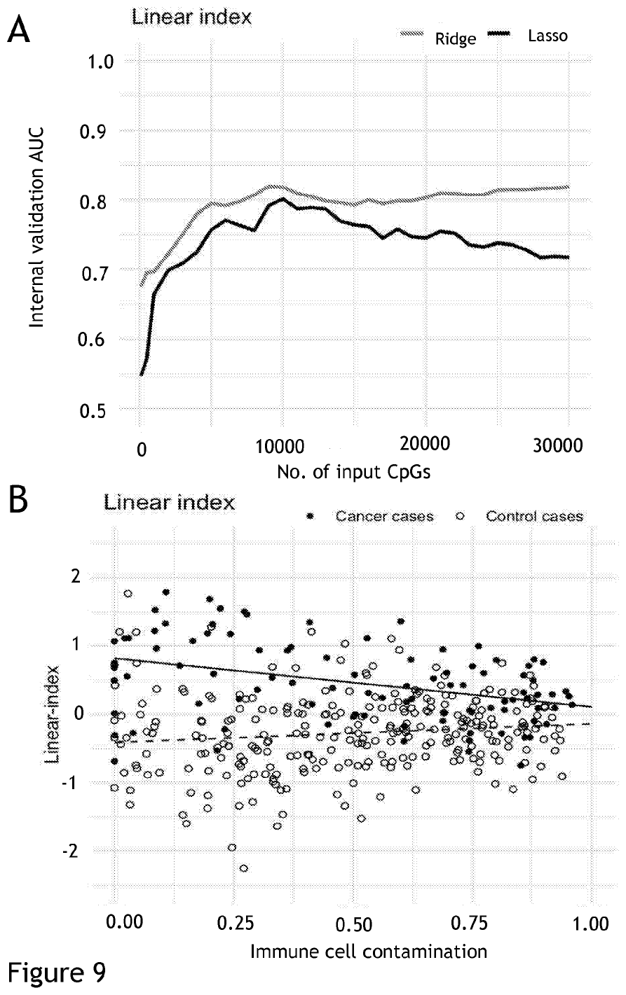

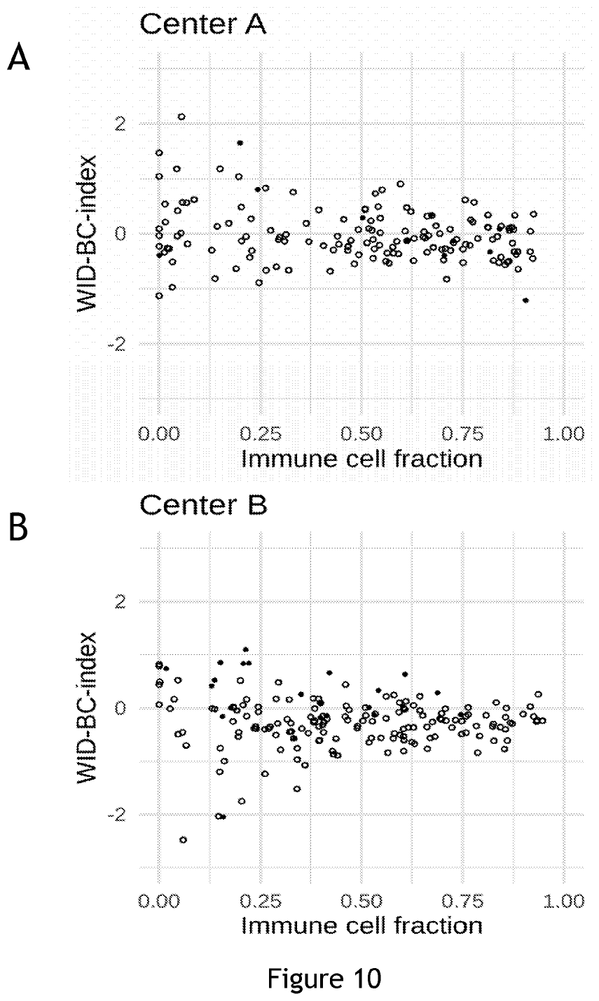

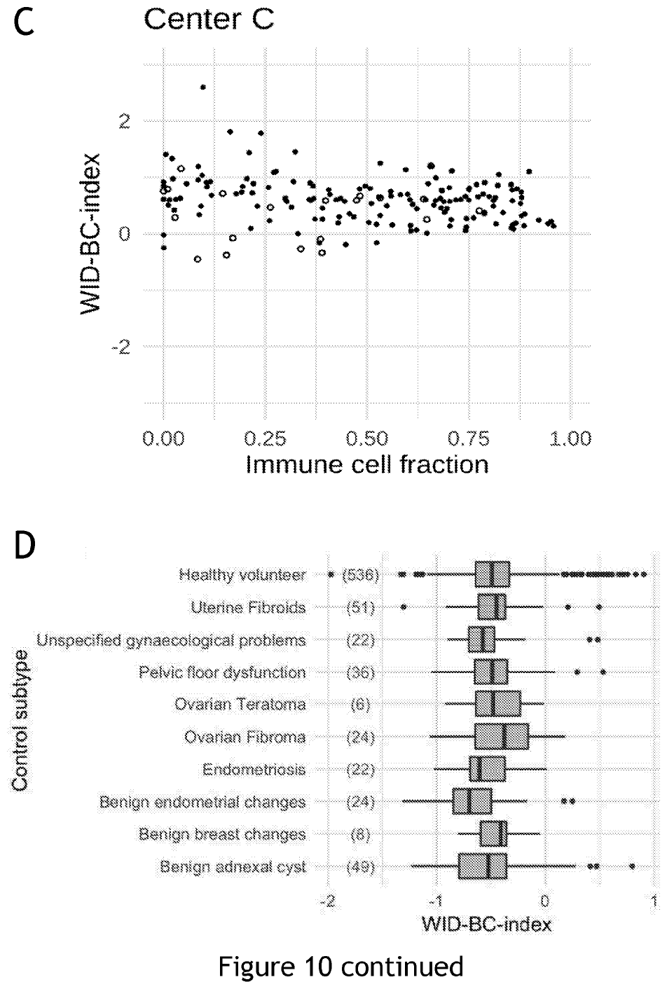

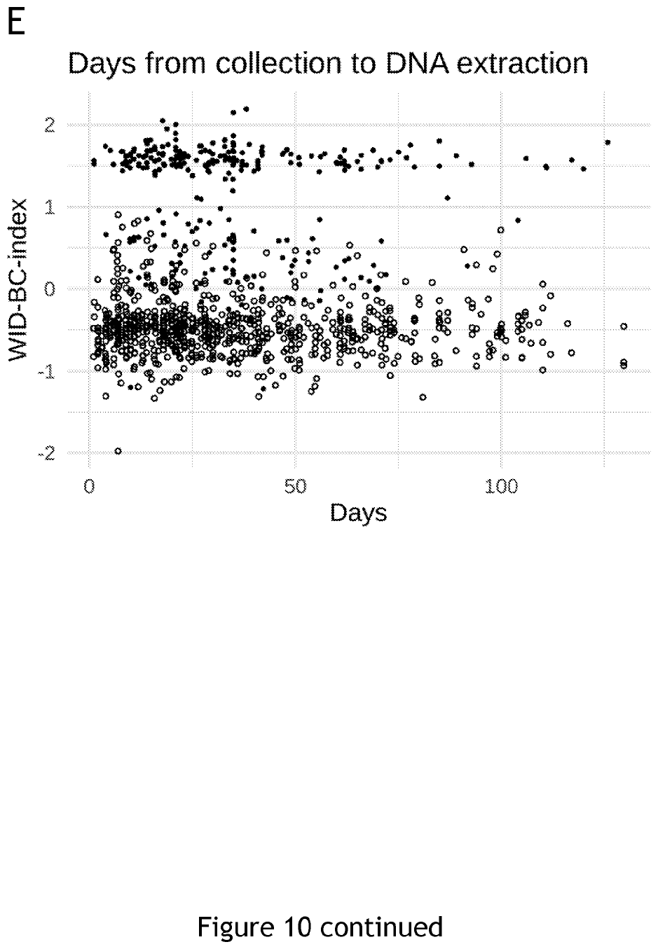

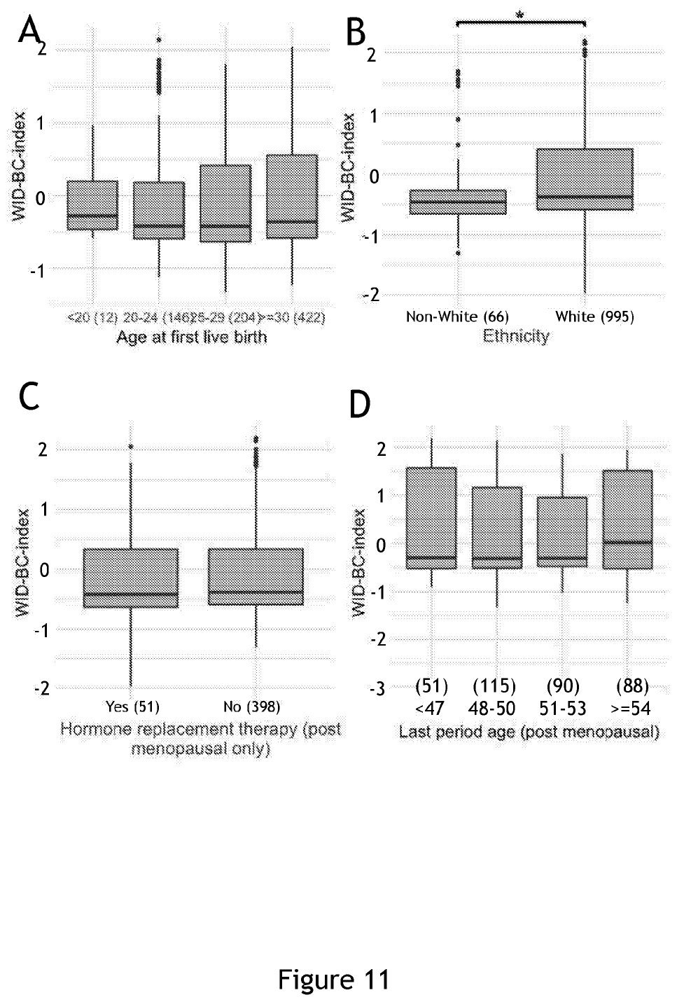

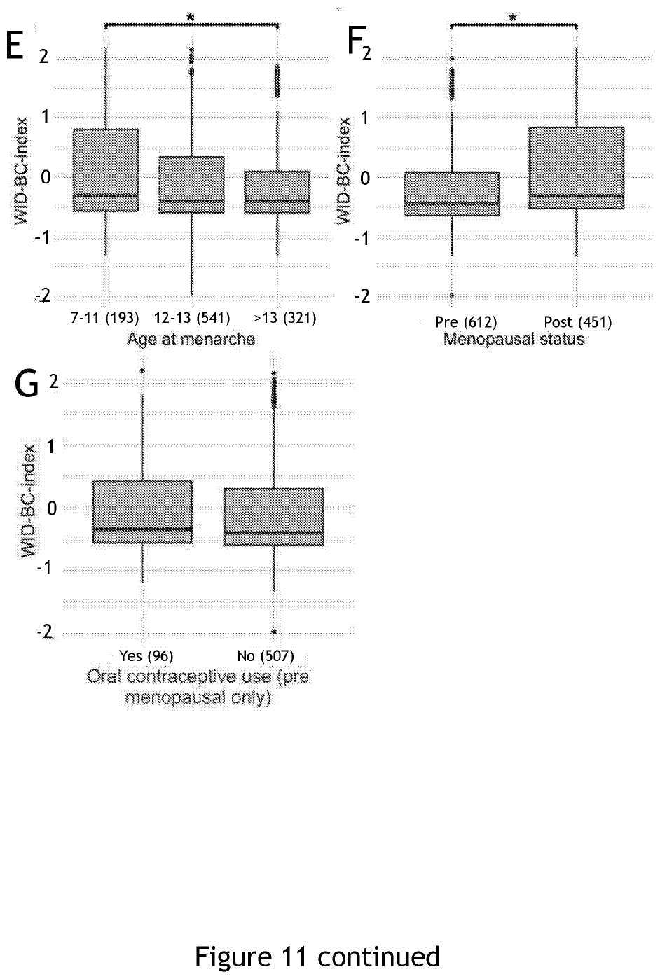

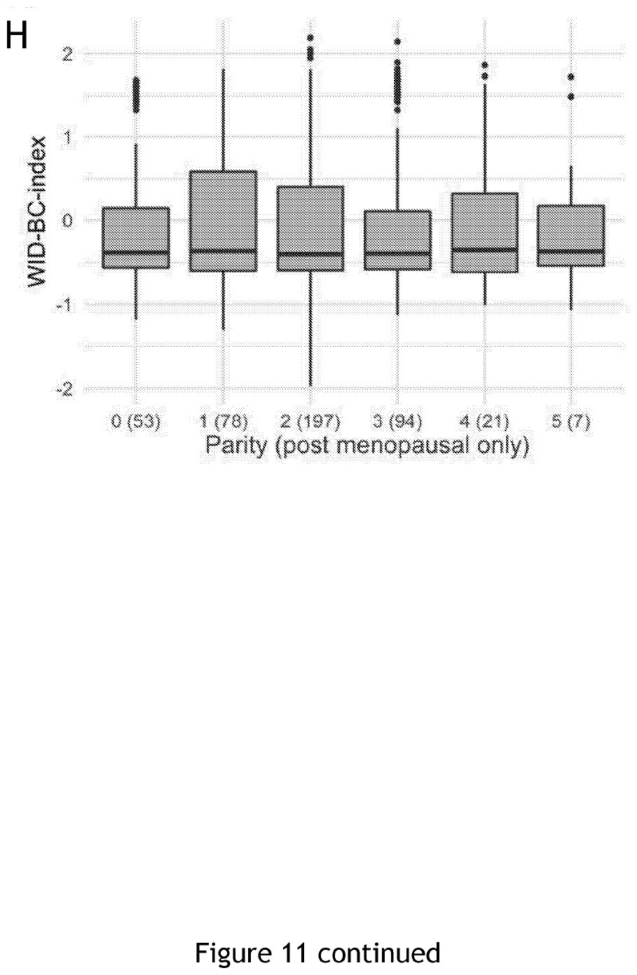

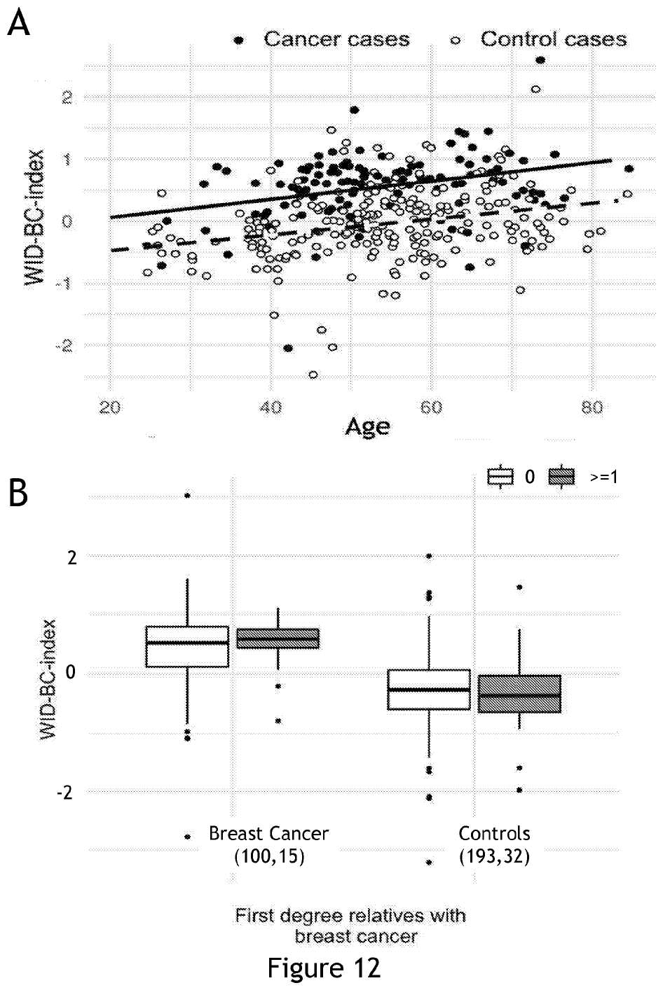

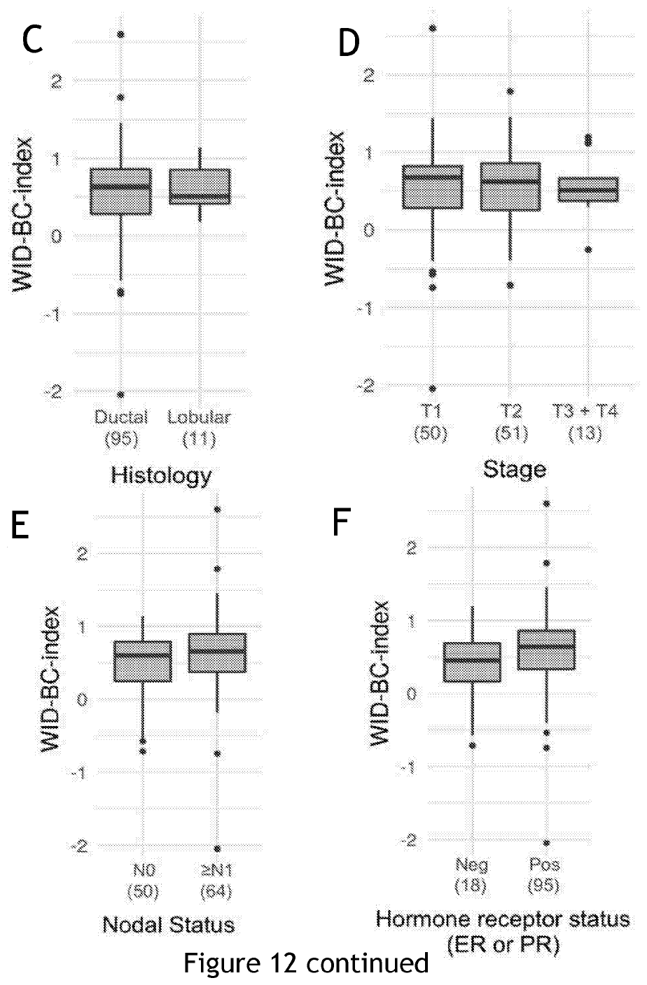

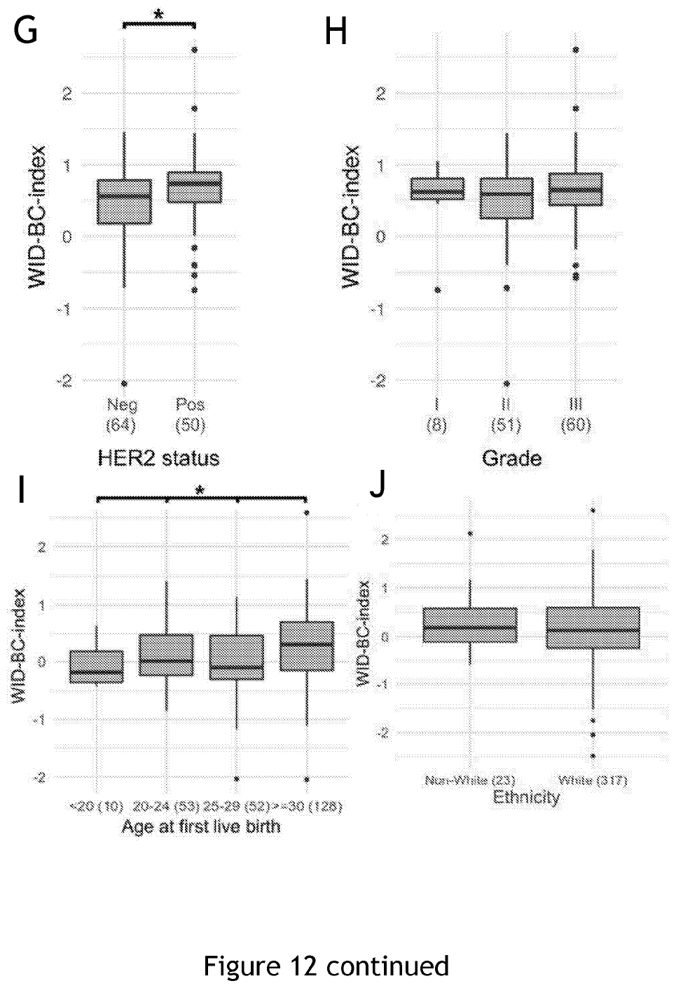

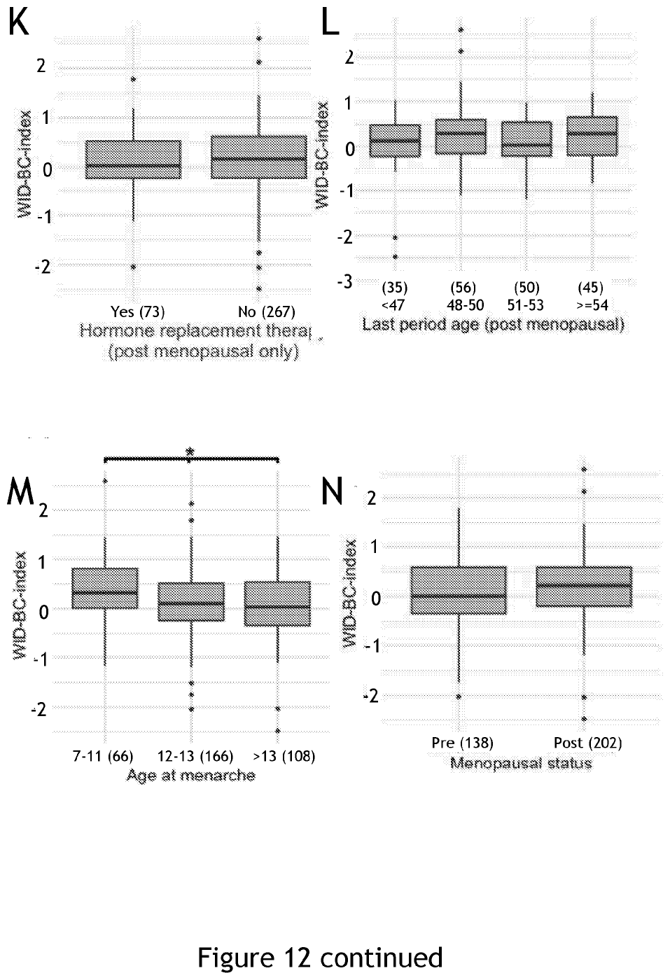

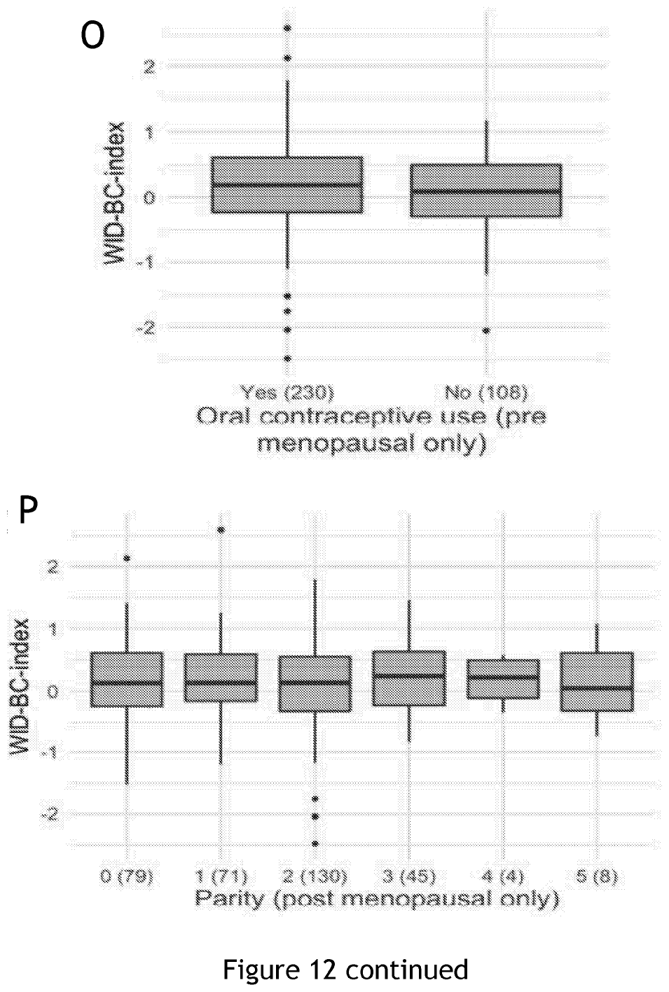

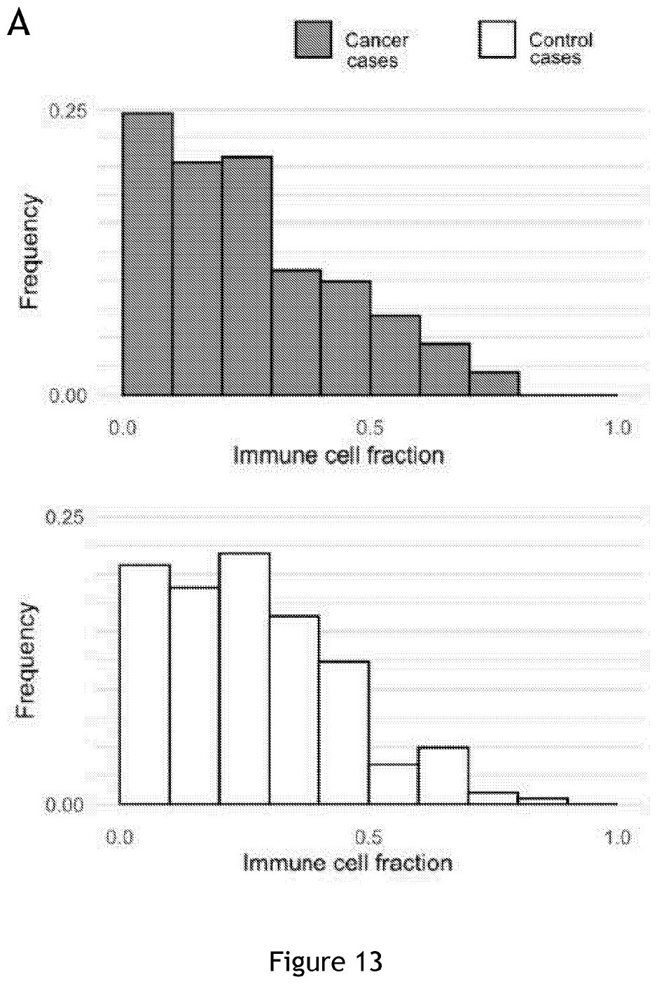

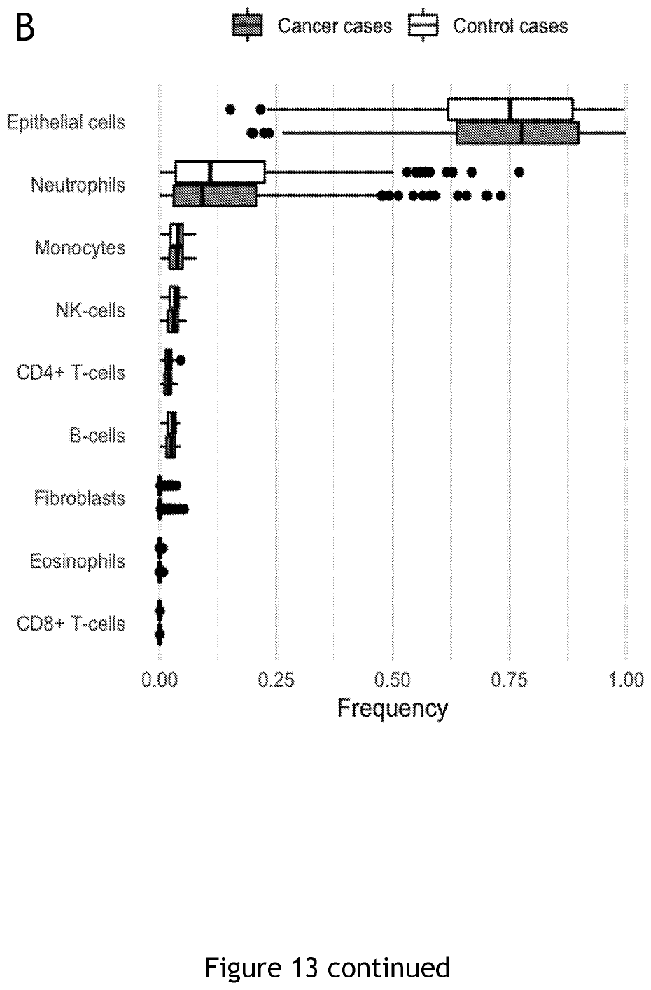

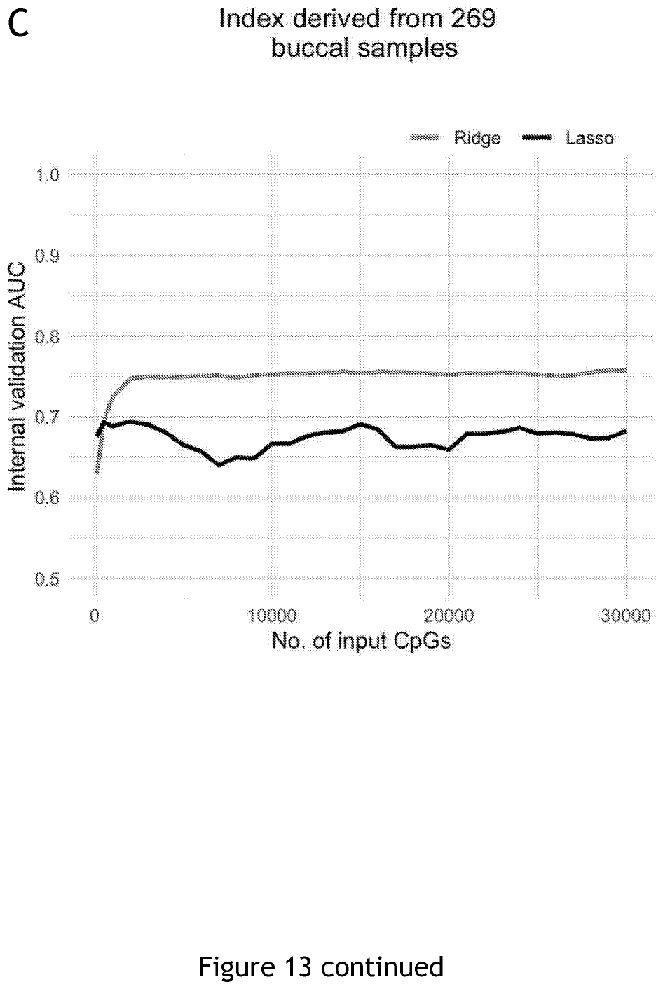

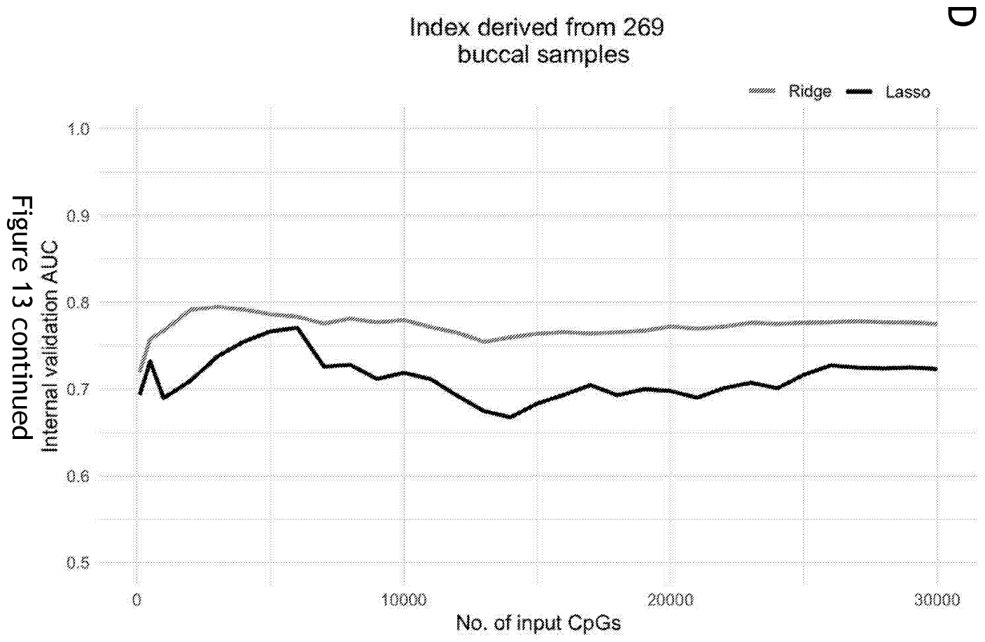

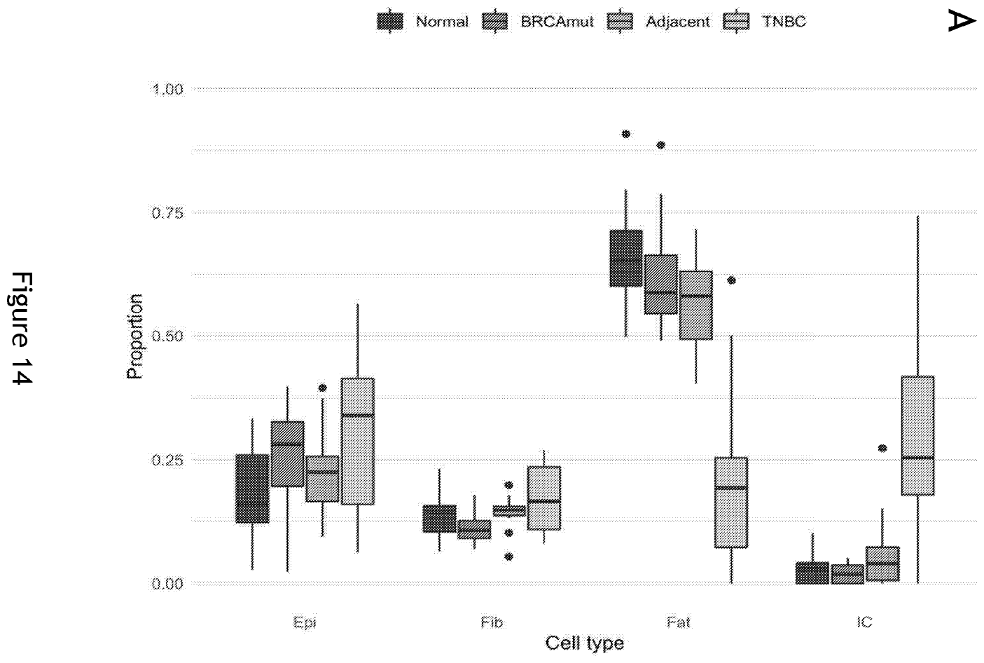

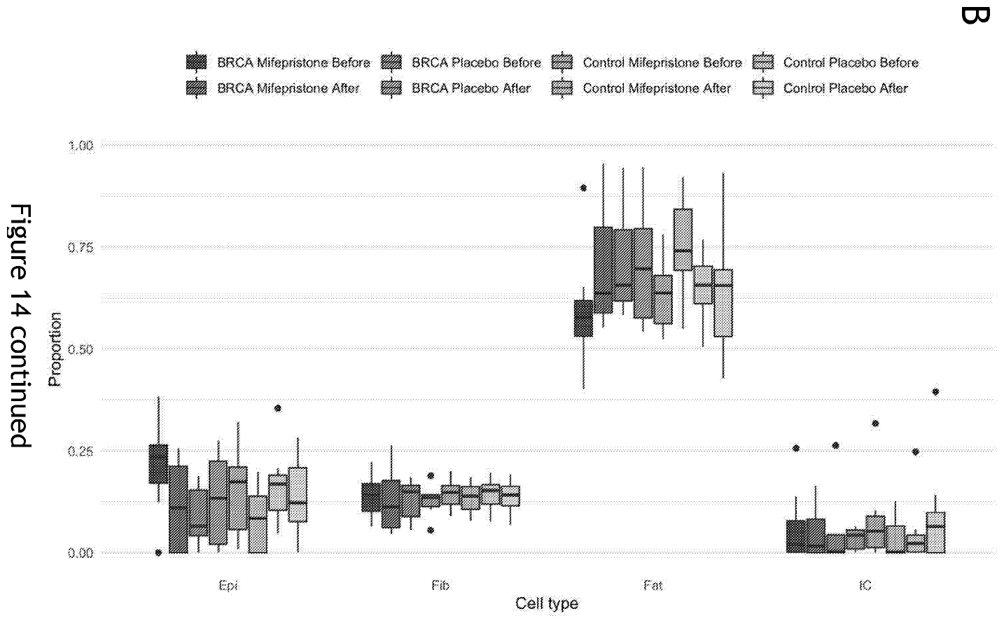

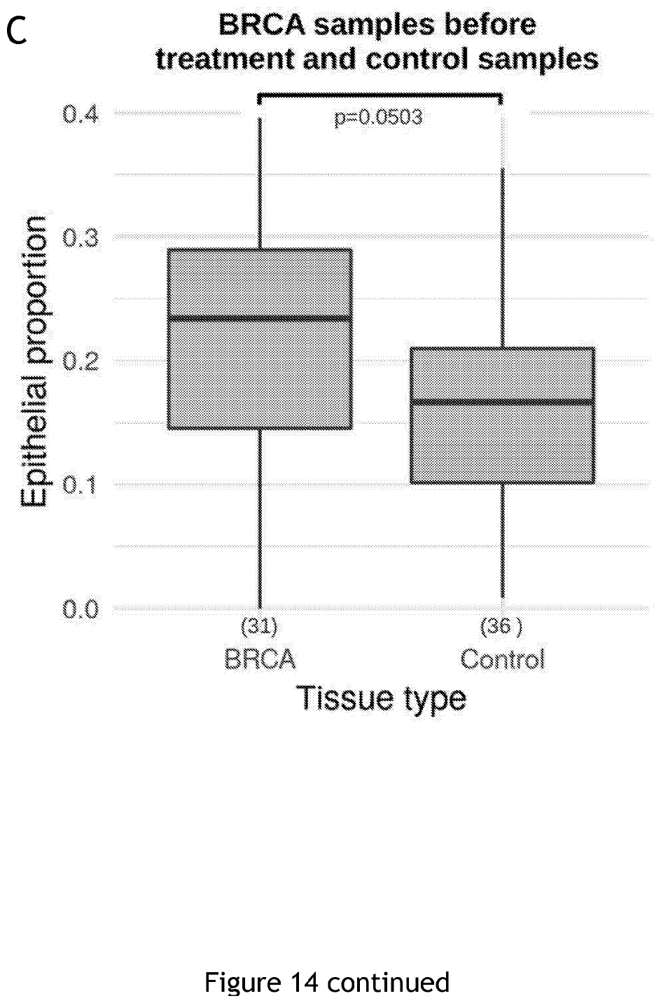

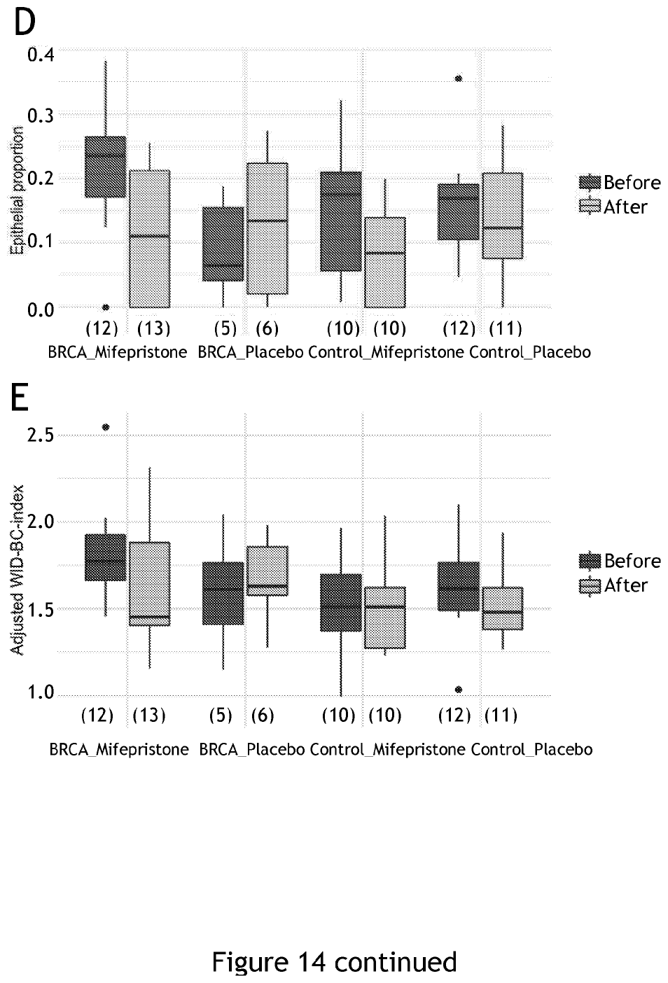

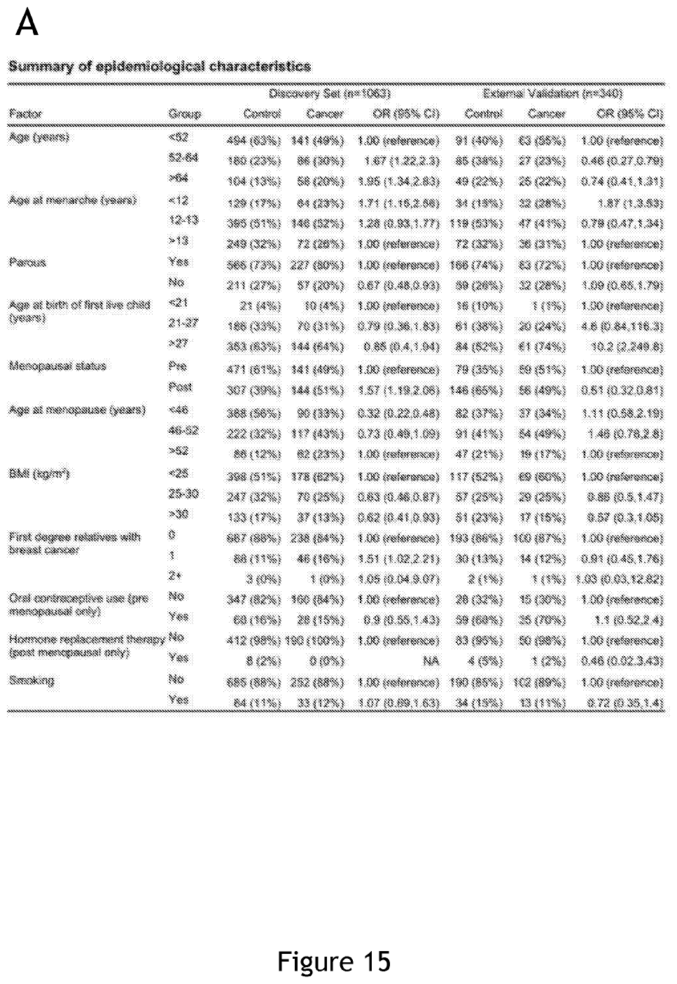

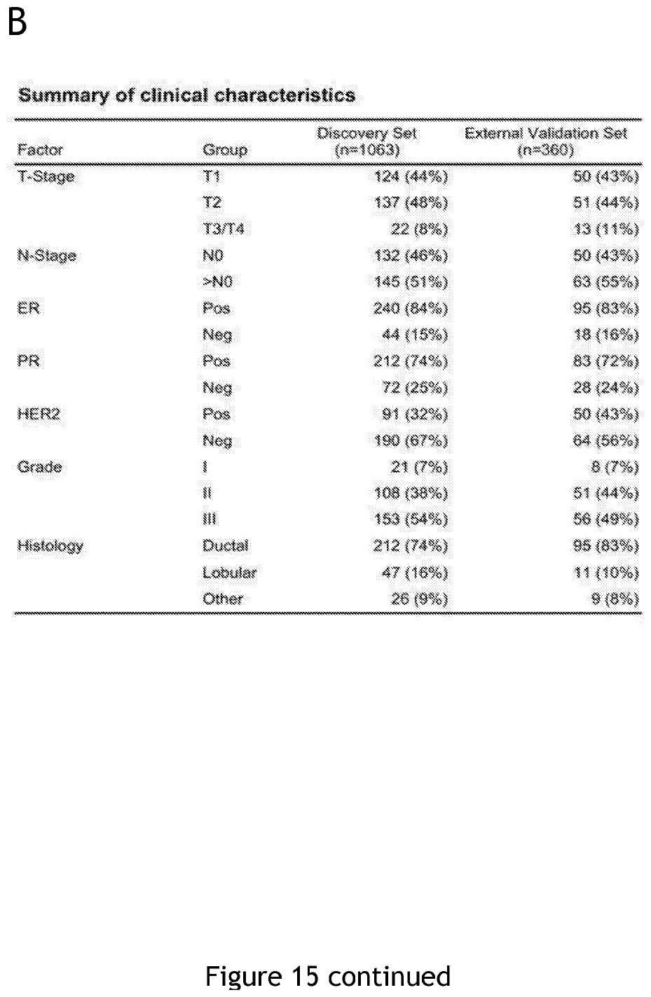

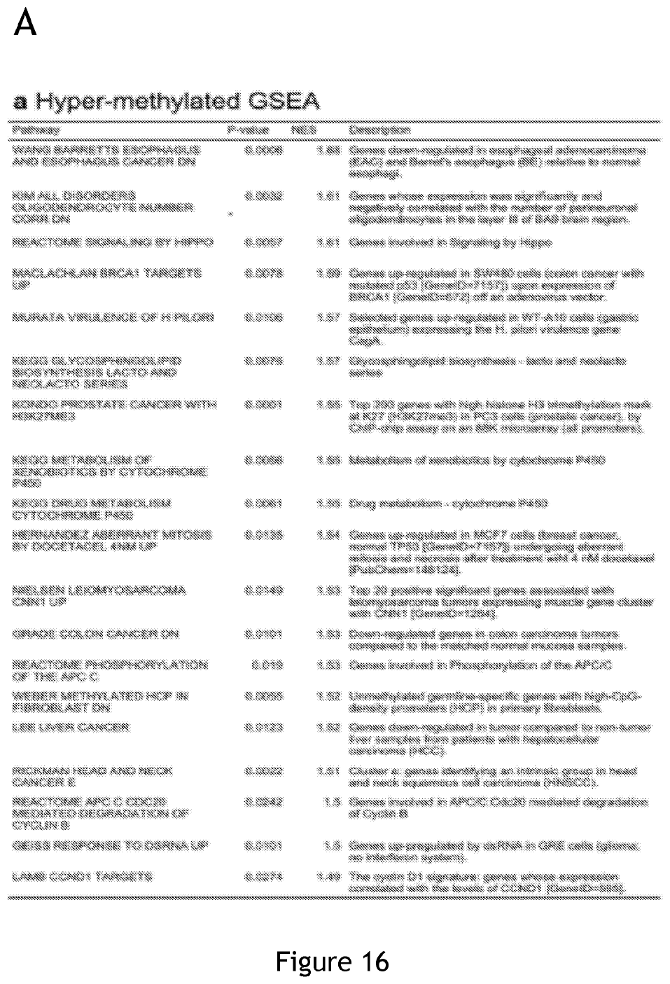

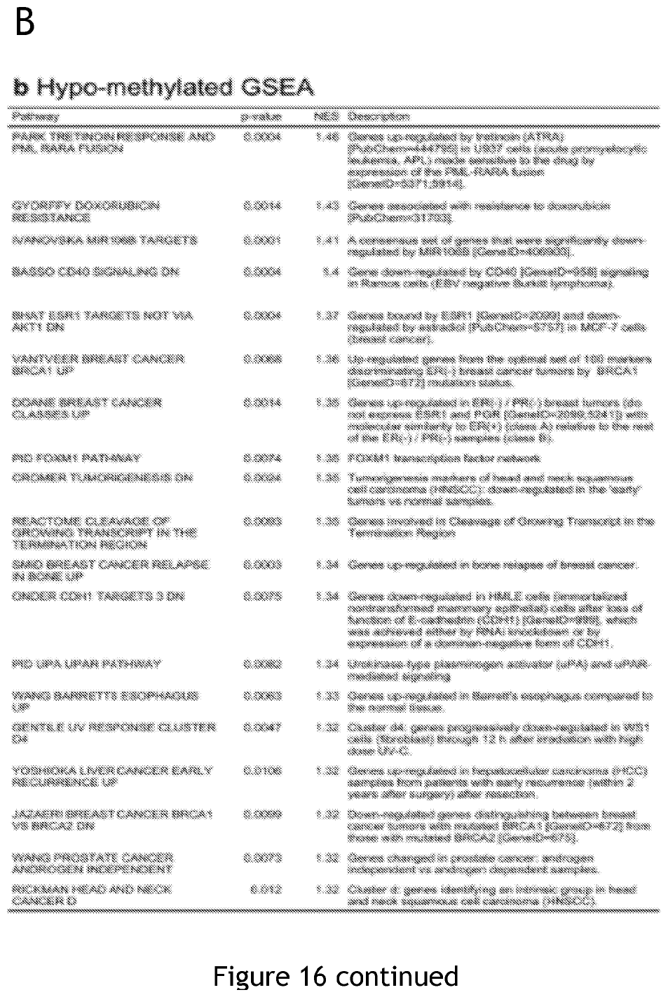

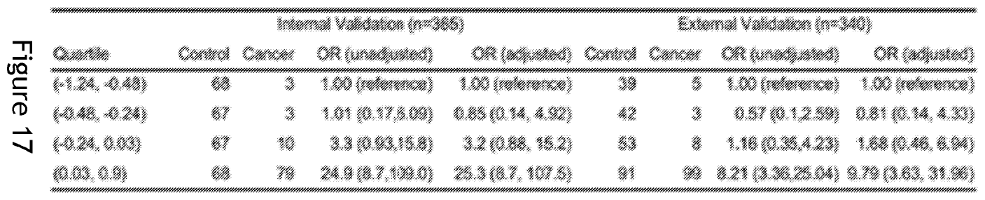

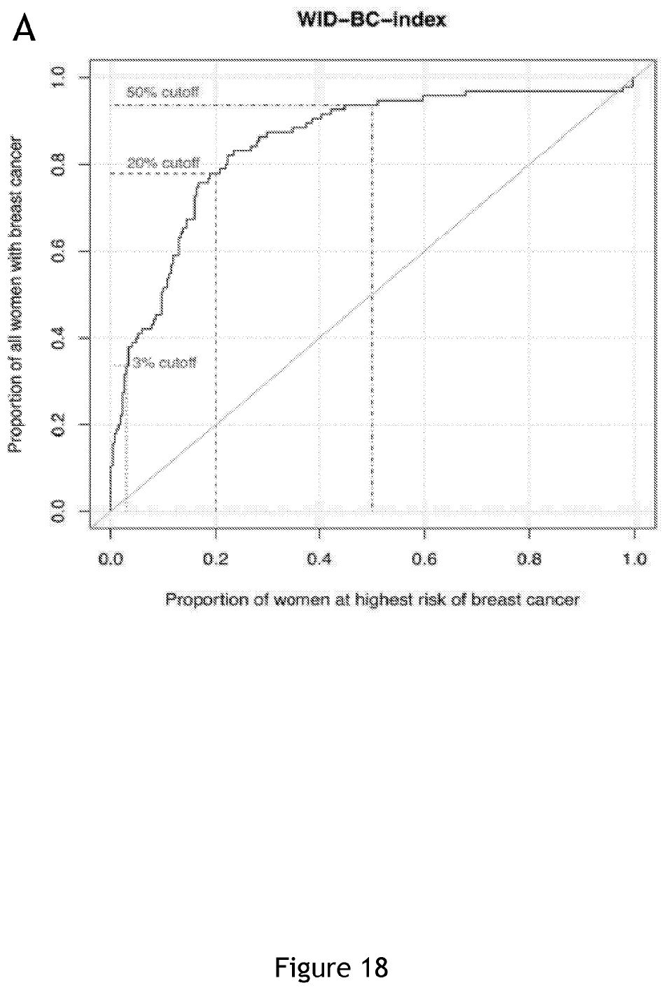

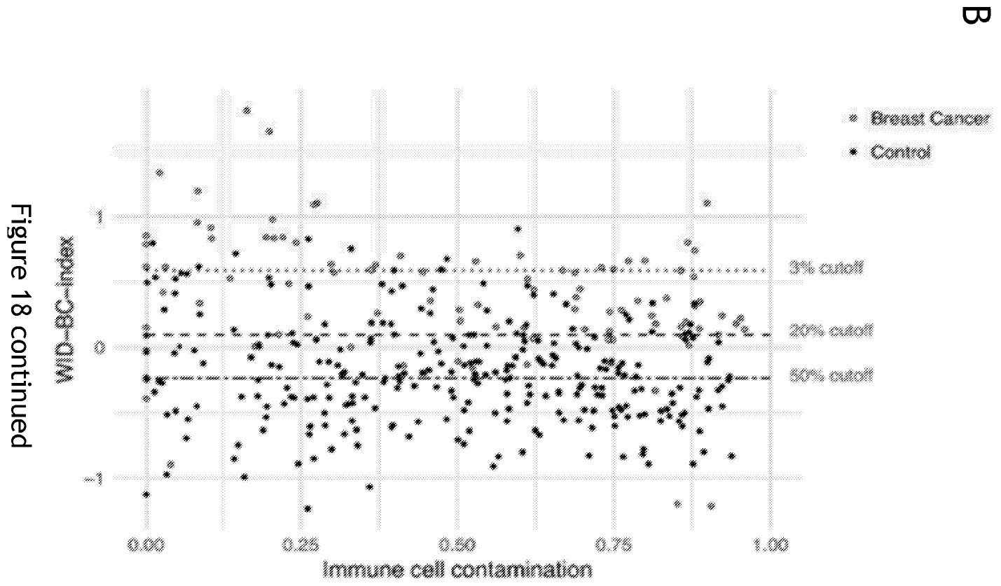

The top n CpGs ranked by epithelial delta-beta estimates were combined with the top n ranked by immune delta-betas and used as inputs to the classifiers. In order to control for the confounding influence of IC, we included non-linear interaction terms (products of IC fraction and beta values) as inputs, thus allowing the classifiers to extract a discriminatory signal that potentially varies with IC. This approach (FIGS. 2A and 2B) was compared to a linear classifier based on the top n epithelial CpGs without interaction terms, however the linear classifier offered consistently inferior performance (FIG. 9).

Predictive performance was evaluated as a function of n using the internal validation dataset (FIG. 2A) and optimal performance of 0.85 (95% CI: 0.80-0.90) was achieved using 54,000 CpGs with ridge regression (FIG. 2B). Since some of the top n CpGs ranked by immune and epithelial delta-beta estimates overlapped, a total of 40,753 unique CpGs were required in order to construct the WID-BC-index. The WID-BC-index was moderately, but significantly associated with IC fraction in the internal validation set (FIG. 2B, linear regression coefficients of −0.47, p=0.003 and −0.22, p=0.02 in cases and controls, respectively). The AUC in the internal validation set equated overall to 0.85 (FIG. 2C), and in samples with an IC fraction ≤0.5 and IC>0.5 was 0.85 and 0.88, respectively.

Further investigation was carried out as to whether there was any association between the WID-BC-index and various technical parameters including the time between sample collection and processing, date of processing, plate number (samples were processed on 96 sample plates) and sentrix position but no significant associations were found. We examined the performance of the WID-BC-index in samples from different study centres, some of which predominantly contributed cases (or controls) to the discovery dataset, but found no evidence that centre was a confounding variable (FIG. 10).

A separate independent external validation dataset consisting of 225 controls and 115 cases was used to validate the index performance (FIG. 15). The WID-BC-index was computed for each woman (FIG. 2D) resulting in an AUC of 0.81 (FIG. 2E; 95% CI: 0.75-0.86). The linear dependence on IC fraction was also present, although only in controls (FIG. 2D, linear regression coefficients of −0.03, p=0.8 and −0.27, p=0.04 in cases and controls respectively).

Ridge regression combines information from all input CpGs in contrast to lasso regression which typically selects a small subset of inputs (an elastic net regression model was also fitted but was found to offer suboptimal performance). Ridge regression offered consistently superior performance suggesting that the discriminatory signal is most robustly extracted by combining a large number of comparatively weak signals from multiple CpG sites. We ranked the 40,754 CpGs used to define the WID-BC-index according to the absolute value of the regression coefficients from the ridge model. In order to assess how important the top CpGs are we trained sub-classifiers on the top n CpGs (FIG. 2F). We observed that AUCs of 0.81 and 0.83 can be achieved with the top 2,000 and 5,000 CpGs respectively indicating that these subsets of CpGs are particularly informative. We also trained sub-classifiers after removing the top n CpGs, and on subsets of 500 CpGs after partitioning the ranked list of CpGs into bins of size 500 (FIG. 2F). In both cases we found that a substantial predictive signal is present in the bottom ranked CpGs. This suggests that the predictive signal is widely distributed among the CpGs used in the WID-BC-index and that there is a high degree of redundancy between them. Interestingly, only 34 CpGs in the training set were significantly associated with case/control status after controlling for age and IC in a linear model and false discovery rate adjustment.

### Example 3

Association with Epidemiological and Clinical Factors

We investigated the relationship between the WID-BC-index and various epidemiological and clinical variables. A modest, but statistically significant association was found between the WID-BC-index and age (FIG. 3A, coefficients of 0.005, p=0.02 and 0.007, p=10⁻⁴ in cases and controls respectively). No significant difference in the WID-BC-index was observed between individuals with 0 and >1 first-degree-relatives with breast cancer (FIG. 3B). The Illumina 650k Infinium Global Screening Array was used to genotype matched blood samples from a subset of 144 cases and 351 controls. We computed a recently published polygenic risk score (PRS; 303 of the 313 SNPs described²² were used) for breast cancer prediction. We found a significant correlation of 0.22 (p=4×10⁻⁵) between the PRS and the WID-BC-index (FIG. 3C). The association was strongest in cancer-free control women (correlation 0.11, p=0.07) compared to cancer cases (correlation −0.03, p=0.7). Patients with T2 cancers had a slightly lower WID-BC-index (FIG. 3D, p=0.02). There were no significant differences according to nodal status, hormone receptor status (positive defined as PR or ER positive), HER2 status, grade, or histology (FIGS. 3E, 3F, 3G, 3H, and 3I). No difference was found between control samples from healthy volunteers and women presenting at hospitals for benign women-specific conditions (FIG. 10). We also tested for associations between the WID-BC-index and age at menarche, age at first live birth, hormone replacement therapy use, oral contraceptive pill use, ethnicity, age of last period, and parity (FIG. 11). Similar results were found for the external validation dataset (FIG. 12).

### Example 4

### Performance of Index in Matched Buccal Samples

DNAme is tissue specific and specific exposures are recorded in certain cell subtypes^{17,23,24}. The majority of cervical epithelial cells are squamous cells and very similar to the epithelial cells found in buccal swabs. In order to assess whether the WID-BC-index (derived from cervical smear samples) can also discriminate breast cancer cases from unaffected controls based on DNAme profiles in buccal samples, we analysed matched buccal samples from a subset of 404 women in the discovery cohort (202 controls and 202 cases). Similar to the cervical smears, a substantial proportion of DNA originates from immune cells (FIG. 13). We found that the discriminatory signal derived using cervical smear samples was also present in these matched buccal samples (FIG. 4A), yielding an AUC of 0.67 (FIG. 4B), although the signal became distorted with a quantitative shift towards more negative values as well as a stronger linear dependence on IC fraction (FIG. 4A). Of the 404 buccal samples, 269 corresponded to the training dataset and 135 to the internal validation dataset. Within the internal validation samples there was a correlation of 0.47 (p<10⁻⁸) between the WID-BC-index computed in matched cervical and buccal samples (FIG. 4C).

A separate index was developed using the buccal samples alone, and a ridge classifier with interaction terms (as described above) was trained on the 269 buccal samples belonging to the training set and validated on the 135 internal validation samples. Optimal performance of 0.75 was obtained based on 4,000 input CpGs (FIG. 13). A second classifier was developed according to the same protocol, but using the 404 matched cervical samples. We observed higher diagnostic performance for the cervical samples with an AUC of 0.79 based on 6,000 input CpGs (performance that is consistent with FIG. 2A).

### Example 5

### The WID-BC-index is Reflective of a Fat-Cell Differentiation

In order to assess whether the WID-BC-index is reflective of a cell-specific program we analysed all ENCODE samples (Table 3) for which EPIC array data were available. We ranked and plotted the WID-BC-index in all primary cell samples and in vitro differentiated cell samples (FIG. 5A). The majority of tissue samples contained substantial proportions of fat, as determined by the Epidish algorithm, and have plotted the index against the fat content of the respective sample (FIG. 5B). Surprisingly we find a very strong direct correlation between the WID-BC-index and the fat content of the sample irrespective of whether the sample was taken from an epithelial or non-epithelial organ. These findings strongly indicate that the WID-BC-index is reflective of a fat cell program.

### Example 6

### WID-BC-Index in Breast Tissue

We, and others, have demonstrated the existence of an epigenetic field defect in the normal breast adjacent to a breast cancer^{9,25}. We therefore wanted to assess whether the WID-BC-index, which was established in cervical smear samples in women with and without a breast cancer, is reflected in normal and cancerous breast tissue. We analysed 14 normal breast samples from healthy women, 14 normal breast samples from women with a BRCA mutation, 14 normal breast samples adjacent to a triple-negative breast cancer, and 14 matched cancer samples. As expected, in contrast to cervical and buccal samples, we found that fat cells constituted a substantial proportion of normal samples and substantially less so for cancerous breast tissue samples (FIG. 14A). As expected, we found in all four sample groups, that the index is substantially higher in fat cells (FIG. 6A) and that this leads to an overall increase in index values in comparison to cervical samples. After linearly adjusting for fat content we observed a distinct trend in which the WID-BC-index increased in tissues that are at increased risk of developing cancer and was highest in the breast cancer (FIG. 6B).

### Example 7

### WID-BC-Index Dynamics Triggered by Cancer Preventive Drug

We analysed breast tissue samples from 21 BRCA carriers and 23 healthy controls before and after treatment with either mifepristone or placebo for two months. In total, 14 BRCA carriers were treated with mifepristone (11 of which provided matched samples) and 7 were given a placebo (4 matched samples). 11 controls were treated with mifepristone (9 matched) and 12 were given a placebo (11 matched). Overall, BRCA carriers had a significantly greater proportion of epithelial cells in comparison to controls (FIGS. 14B and 14C). After treatment with mifepristone (but not in the placebo group) we observed a statistically significant decrease in epithelial cell proportion in both the matched BRCA and control groups (FIG. 6C and FIG. 14D). After adjustment for fat content we also observed a downward trend in the WID-BC-index in 80% of the mifepristone treated women and only in 60% of the placebo treated controls, although this did not reach statistical significance (FIG. 6D, see FIG. 14E for matched and unmatched samples combined).

### Example 8

### Discussion

We have identified a cervical smear based DNAme signature (the WID-BC-index) which provides an unprecedented opportunity to identify women with a primary breast cancer with poor prognosis features based on a bio-sample which has no direct (anatomical) link to the diseased organ (i.e. women in the top quartile of the WID-BC-Index have ˜10 fold increased risk for breast cancer independent of any other risk factors; FIG. 17). The fact that the WID-BC-index discovered in a cervical smear sample (i) does not increase with tumour size or surrogates for dissemination (i.e. nodal metastasis), (ii) increases in normal breast samples with increasing tendency towards cancer and is highest in the cancer tissue and (iii) is reflective of a fat cell epigenetic program strongly supports the view that cervical DNAme reflects breast cancer predisposition rather than purely the current presence of an established breast cancer. Our findings described here are consistent with data published more than 30 years ago showing that patients with hereditary breast cancer and their first degree relatives harbour a differentiation defect²⁶.

Considerable effort in the past has shown that by combining SNPs, mammographic density, and epidemiologic risk factors the AUC that predicts breast cancer can be increased up to 0.68⁶. Studying population-based cervical smear samples from women who develop a breast cancer several years after sample donations will be a prerequisite in order to assess whether the actual risk-predictive nature of the WID-BC-index will continue to outperform current breast cancer predictive algorithms. Whether DNAme profiles assessed in cervical smear samples, and/or in breast samples, can act as surrogates for monitoring breast cancer preventive measures will need to be assessed in prospective clinical trials.

TABLE 1
Table 1 below provides exemplary μ and σ real valued parameters
derived using the data and methods set out in the Examples
herein for CpG subsets identified in SEQ ID NOs 1 to 40,753

| CpG subset | mu | sigma |
|---|---|---|
| 1-500 | 11.88 | 2.65 |
| 1-1000 | 15.42 | 4.32 |
| 1-1500 | 11.43 | 3.94 |
| 1-2000 | 5.23 | 2.49 |
| 1-2500 | 4.91 | 2.6 |
| 1-3000 | 4.13 | 2.67 |
| 1-3500 | 7.96 | 5.02 |
| 1-4000 | 6.83 | 4.36 |
| 1-4500 | 5.19 | 4.02 |
| 1-5000 | 6.39 | 4.84 |
| 1-5500 | 4.49 | 3.42 |
| 1-6000 | 5.11 | 4.13 |
| 1-6500 | 4.23 | 4.01 |
| 1-7000 | 3.98 | 4.49 |
| 1-7500 | 4.21 | 4.44 |
| 1-8000 | 4.15 | 4.92 |
| 1-8500 | 3.63 | 4.48 |
| 1-9000 | 3.2 | 4.88 |
| 1-9500 | 3.37 | 5.45 |
| 1-10000 | 2.74 | 5.46 |
| 1-11000 | 2.2 | 5.41 |
| 1-12000 | 1.75 | 5.43 |
| 1-13000 | 1.7 | 5.14 |
| 1-14000 | 1.66 | 5.47 |
| 1-15000 | 1.77 | 5.33 |
| 1-16000 | 1.87 | 5.58 |
| 1-17000 | 2.2 | 5.59 |
| 1-18000 | 2.34 | 5.6 |
| 1-19000 | 2.58 | 5.53 |
| 1-20000 | 2.56 | 5.46 |
| 1-22000 | 2.67 | 5.63 |
| 1-24000 | 2.77 | 5.65 |
| 1-26000 | 2.69 | 5.57 |
| 1-28000 | 2.89 | 5.66 |
| 1-30000 | 3.09 | 5.59 |
| 1-32000 | 3.13 | 5.67 |
| 1-34000 | 3.12 | 5.59 |
| 1-36000 | 3.1 | 5.52 |
| 1-38000 | 3.15 | 5.67 |
| 1-40753 | 3.03 | 5.52 |

TABLE 4
Table 4 below provides exemplary AUC, sensitivity and specificity derived using the
data and methods set out in the Examples for CpG subsets identified in SEQ ID NOs 1
to 40,753 and set out in the left hand column of Table 4

| CpG | AUC (internal | Sensitivity | Specificity | Sensitivity | Specificity | Sensitivity | Specificity |
|---|---|---|---|---|---|---|---|
| subset | validation) | (q = 0.587) | (q = 0.587) | (q = 0.09) | (q = 0.09) | (q = −0.235) | (q = −0.235) |
| 1-500 | 0.73 | 0.26 | 0.93 | 0.63 | 0.69 | 0.85 | 0.52 |
| 1-1000 | 0.77 | 0.29 | 0.95 | 0.68 | 0.73 | 0.88 | 0.49 |
| 1-1500 | 0.8 | 0.29 | 0.94 | 0.73 | 0.75 | 0.92 | 0.51 |
| 1-2000 | 0.81 | 0.33 | 0.94 | 0.69 | 0.78 | 0.94 | 0.51 |
| 1-2500 | 0.82 | 0.31 | 0.95 | 0.68 | 0.78 | 0.93 | 0.51 |
| 1-3000 | 0.82 | 0.28 | 0.94 | 0.71 | 0.78 | 0.93 | 0.51 |
| 1-3500 | 0.83 | 0.26 | 0.95 | 0.72 | 0.79 | 0.94 | 0.49 |
| 1-4000 | 0.83 | 0.29 | 0.95 | 0.69 | 0.8 | 0.95 | 0.47 |
| 1-4500 | 0.83 | 0.32 | 0.94 | 0.72 | 0.79 | 0.94 | 0.48 |
| 1-5000 | 0.84 | 0.29 | 0.96 | 0.72 | 0.8 | 0.96 | 0.49 |
| 1-5500 | 0.83 | 0.27 | 0.95 | 0.73 | 0.79 | 0.94 | 0.49 |
| 1-6000 | 0.83 | 0.27 | 0.96 | 0.73 | 0.79 | 0.95 | 0.5 |
| 1-6500 | 0.84 | 0.31 | 0.96 | 0.71 | 0.79 | 0.95 | 0.49 |
| 1-7000 | 0.84 | 0.29 | 0.96 | 0.73 | 0.8 | 0.95 | 0.5 |
| 1-7500 | 0.84 | 0.29 | 0.96 | 0.72 | 0.8 | 0.95 | 0.52 |
| 1-8000 | 0.84 | 0.29 | 0.96 | 0.74 | 0.79 | 0.95 | 0.51 |
| 1-8500 | 0.84 | 0.29 | 0.96 | 0.73 | 0.8 | 0.94 | 0.53 |
| 1-9000 | 0.84 | 0.29 | 0.96 | 0.75 | 0.79 | 0.95 | 0.52 |
| 1-9500 | 0.84 | 0.28 | 0.95 | 0.74 | 0.78 | 0.95 | 0.5 |
| 1-10000 | 0.84 | 0.29 | 0.95 | 0.74 | 0.8 | 0.95 | 0.5 |
| 1-11000 | 0.84 | 0.29 | 0.96 | 0.73 | 0.8 | 0.95 | 0.51 |
| 1-12000 | 0.84 | 0.28 | 0.96 | 0.74 | 0.8 | 0.95 | 0.52 |
| 1-13000 | 0.84 | 0.28 | 0.96 | 0.74 | 0.8 | 0.95 | 0.52 |
| 1-14000 | 0.84 | 0.27 | 0.96 | 0.76 | 0.8 | 0.95 | 0.52 |
| 1-15000 | 0.84 | 0.28 | 0.96 | 0.74 | 0.8 | 0.95 | 0.52 |
| 1-16000 | 0.84 | 0.27 | 0.96 | 0.74 | 0.8 | 0.95 | 0.53 |
| 1-17000 | 0.85 | 0.32 | 0.96 | 0.74 | 0.8 | 0.95 | 0.51 |
| 1-18000 | 0.85 | 0.33 | 0.96 | 0.76 | 0.8 | 0.95 | 0.51 |
| 1-19000 | 0.85 | 0.34 | 0.96 | 0.77 | 0.8 | 0.94 | 0.51 |
| 1-20000 | 0.85 | 0.34 | 0.96 | 0.76 | 0.8 | 0.94 | 0.51 |
| 1-22000 | 0.85 | 0.34 | 0.97 | 0.77 | 0.8 | 0.94 | 0.51 |
| 1-24000 | 0.85 | 0.33 | 0.97 | 0.77 | 0.8 | 0.94 | 0.51 |
| 1-26000 | 0.85 | 0.34 | 0.97 | 0.77 | 0.8 | 0.94 | 0.51 |
| 1-28000 | 0.85 | 0.34 | 0.97 | 0.77 | 0.8 | 0.94 | 0.51 |
| 1-30000 | 0.85 | 0.33 | 0.97 | 0.77 | 0.8 | 0.94 | 0.51 |
| 1-32000 | 0.85 | 0.32 | 0.97 | 0.77 | 0.8 | 0.94 | 0.5 |
| 1-34000 | 0.85 | 0.33 | 0.97 | 0.78 | 0.8 | 0.94 | 0.5 |
| 1-36000 | 0.85 | 0.33 | 0.97 | 0.78 | 0.8 | 0.94 | 0.5 |
| 1-38000 | 0.85 | 0.33 | 0.97 | 0.77 | 0.8 | 0.94 | 0.5 |
| 1-40753 | 0.85 | 0.34 | 0.97 | 0.78 | 0.8 | 0.94 | 0.5 |

TABLE 5
Table 5 below provides exemplary AUC, sensitivity and specificity derived using the
data and methods set out in the Examples herein for CpG subsets identified in SEQ ID NOs 1
to 40,753 and set out in the left hand column of Table 5

| CpG | AUC (internal | Sensitivity | Specificity | Sensitivity | Specificity | Sensitivity | Specificity |
|---|---|---|---|---|---|---|---|
| subset | validation) | (q = 0.587) | (q = 0.587) | (q = 0.09) | (q = 0.09) | (q = −0.235) | (q = −0.235) |
| 500-40753 | 0.84 | 0.35 | 0.97 | 0.77 | 0.79 | 0.94 | 0.51 |
| 1000-40753 | 0.84 | 0.34 | 0.96 | 0.79 | 0.78 | 0.92 | 0.51 |
| 1500-40753 | 0.81 | 0.38 | 0.94 | 0.73 | 0.74 | 0.91 | 0.53 |
| 2000-40753 | 0.81 | 0.39 | 0.94 | 0.75 | 0.74 | 0.91 | 0.52 |
| 2500-40753 | 0.8 | 0.41 | 0.92 | 0.72 | 0.74 | 0.91 | 0.54 |
| 3000-40753 | 0.8 | 0.4 | 0.91 | 0.72 | 0.74 | 0.91 | 0.54 |
| 3500-40753 | 0.79 | 0.42 | 0.9 | 0.73 | 0.74 | 0.87 | 0.56 |
| 4000-40753 | 0.78 | 0.41 | 0.9 | 0.69 | 0.74 | 0.86 | 0.56 |
| 4500-40753 | 0.78 | 0.42 | 0.9 | 0.71 | 0.74 | 0.86 | 0.57 |
| 5000-40753 | 0.77 | 0.42 | 0.89 | 0.69 | 0.74 | 0.86 | 0.56 |
| 5500-40753 | 0.76 | 0.36 | 0.89 | 0.66 | 0.74 | 0.8 | 0.57 |
| 6000-40753 | 0.74 | 0.33 | 0.89 | 0.61 | 0.75 | 0.78 | 0.57 |
| 6500-40753 | 0.74 | 0.32 | 0.89 | 0.61 | 0.75 | 0.78 | 0.57 |
| 7000-40753 | 0.74 | 0.32 | 0.89 | 0.6 | 0.75 | 0.78 | 0.57 |
| 7500-40753 | 0.74 | 0.33 | 0.88 | 0.61 | 0.74 | 0.77 | 0.57 |
| 8000-40753 | 0.73 | 0.32 | 0.88 | 0.59 | 0.74 | 0.77 | 0.56 |
| 8500-40753 | 0.73 | 0.32 | 0.86 | 0.6 | 0.74 | 0.78 | 0.56 |
| 9000-40753 | 0.72 | 0.31 | 0.87 | 0.58 | 0.75 | 0.75 | 0.56 |
| 9500-40753 | 0.72 | 0.31 | 0.87 | 0.58 | 0.75 | 0.75 | 0.57 |
| 10000-40753 | 0.72 | 0.29 | 0.86 | 0.58 | 0.74 | 0.76 | 0.57 |
| 11000-40753 | 0.71 | 0.29 | 0.86 | 0.58 | 0.75 | 0.76 | 0.57 |
| 12000-40753 | 0.71 | 0.28 | 0.86 | 0.59 | 0.75 | 0.76 | 0.59 |
| 13000-40753 | 0.71 | 0.29 | 0.86 | 0.59 | 0.74 | 0.75 | 0.59 |
| 14000-40753 | 0.68 | 0.27 | 0.87 | 0.53 | 0.75 | 0.68 | 0.59 |
| 15000-40753 | 0.69 | 0.28 | 0.87 | 0.54 | 0.74 | 0.69 | 0.58 |
| 16000-40753 | 0.68 | 0.26 | 0.87 | 0.53 | 0.75 | 0.65 | 0.6 |
| 17000-40753 | 0.67 | 0.26 | 0.87 | 0.47 | 0.75 | 0.65 | 0.6 |
| 18000-40753 | 0.67 | 0.26 | 0.87 | 0.47 | 0.75 | 0.64 | 0.61 |
| 19000-40753 | 0.66 | 0.25 | 0.88 | 0.43 | 0.75 | 0.63 | 0.61 |
| 20000-40753 | 0.66 | 0.25 | 0.88 | 0.43 | 0.76 | 0.63 | 0.6 |
| 22000-40753 | 0.67 | 0.25 | 0.87 | 0.49 | 0.74 | 0.65 | 0.61 |
| 24000-40753 | 0.67 | 0.25 | 0.86 | 0.49 | 0.74 | 0.67 | 0.61 |
| 26000-40753 | 0.67 | 0.25 | 0.86 | 0.51 | 0.75 | 0.67 | 0.58 |
| 28000-40753 | 0.64 | 0.24 | 0.88 | 0.43 | 0.76 | 0.63 | 0.6 |
| 30000-40753 | 0.63 | 0.25 | 0.88 | 0.39 | 0.76 | 0.63 | 0.6 |
| 32000-40753 | 0.62 | 0.24 | 0.87 | 0.38 | 0.76 | 0.61 | 0.59 |
| 34000-40753 | 0.62 | 0.24 | 0.87 | 0.37 | 0.76 | 0.63 | 0.59 |
| 36000-40753 | 0.61 | 0.24 | 0.87 | 0.37 | 0.77 | 0.6 | 0.6 |
| 38000-40753 | 0.61 | 0.24 | 0.87 | 0.35 | 0.76 | 0.61 | 0.58 |

TABLE 6
Table 6 below provides exemplary AUC, sensitivity and specificity derived using the
data and methods set out in the Examples herein for CpG subsets identified in SEQ ID NOs 1
to 40,753 and set out in the left hand column of Table 6

| CpG | AUC (internal | Sensitivity | Specificity | Sensitivity | Specificity | Sensitivity | Specificity |
|---|---|---|---|---|---|---|---|
| subset | validation) | (q = 0.587) | (q = 0.587) | (q = 0.09) | (q = 0.09) | (q = −0.235) | (q = −0.235) |
| 1-500 | 0.73 | 0.26 | 0.93 | 0.63 | 0.69 | 0.85 | 0.52 |
| 501-1000 | 0.74 | 0.35 | 0.9 | 0.66 | 0.67 | 0.86 | 0.49 |
| 1001-1500 | 0.75 | 0.39 | 0.9 | 0.67 | 0.74 | 0.81 | 0.5 |
| 1501-2000 | 0.72 | 0.34 | 0.91 | 0.58 | 0.72 | 0.79 | 0.51 |
| 2001-2500 | 0.72 | 0.34 | 0.89 | 0.68 | 0.68 | 0.81 | 0.48 |
| 2501-3000 | 0.69 | 0.33 | 0.87 | 0.63 | 0.67 | 0.78 | 0.47 |
| 3001-3500 | 0.73 | 0.46 | 0.89 | 0.65 | 0.66 | 0.79 | 0.48 |
| 3501-4000 | 0.69 | 0.37 | 0.89 | 0.64 | 0.67 | 0.72 | 0.46 |
| 4001-4500 | 0.73 | 0.46 | 0.84 | 0.71 | 0.64 | 0.81 | 0.47 |
| 4501-5000 | 0.7 | 0.39 | 0.89 | 0.62 | 0.66 | 0.76 | 0.44 |
| 5001-5500 | 0.71 | 0.39 | 0.86 | 0.66 | 0.63 | 0.82 | 0.45 |
| 5501-6000 | 0.7 | 0.32 | 0.91 | 0.57 | 0.74 | 0.73 | 0.53 |
| 6001-6500 | 0.76 | 0.47 | 0.88 | 0.71 | 0.71 | 0.84 | 0.5 |
| 6501-7000 | 0.76 | 0.46 | 0.87 | 0.72 | 0.68 | 0.86 | 0.49 |
| 7001-7500 | 0.66 | 0.27 | 0.87 | 0.54 | 0.73 | 0.75 | 0.5 |
| 7501-8000 | 0.76 | 0.48 | 0.87 | 0.76 | 0.69 | 0.85 | 0.49 |
| 8001-8500 | 0.69 | 0.29 | 0.87 | 0.62 | 0.67 | 0.78 | 0.51 |
| 8501-9000 | 0.73 | 0.37 | 0.88 | 0.62 | 0.69 | 0.82 | 0.53 |
| 9001-9500 | 0.72 | 0.38 | 0.84 | 0.64 | 0.69 | 0.78 | 0.54 |
| 9501-10000 | 0.69 | 0.28 | 0.88 | 0.62 | 0.7 | 0.75 | 0.55 |
| 10001-10500 | 0.69 | 0.31 | 0.87 | 0.63 | 0.69 | 0.8 | 0.48 |
| 10501-11000 | 0.71 | 0.33 | 0.92 | 0.59 | 0.72 | 0.75 | 0.56 |
| 11001-11500 | 0.78 | 0.54 | 0.86 | 0.73 | 0.64 | 0.86 | 0.51 |
| 11501-12000 | 0.67 | 0.41 | 0.81 | 0.65 | 0.64 | 0.76 | 0.45 |
| 12001-12500 | 0.72 | 0.39 | 0.87 | 0.6 | 0.7 | 0.74 | 0.54 |
| 12501-13000 | 0.73 | 0.33 | 0.89 | 0.66 | 0.68 | 0.8 | 0.53 |
| 13001-13500 | 0.73 | 0.45 | 0.87 | 0.67 | 0.69 | 0.77 | 0.56 |
| 13501-14000 | 0.74 | 0.45 | 0.87 | 0.64 | 0.69 | 0.75 | 0.55 |
| 14001-14500 | 0.73 | 0.42 | 0.87 | 0.62 | 0.69 | 0.74 | 0.55 |
| 14501-15000 | 0.72 | 0.39 | 0.85 | 0.67 | 0.68 | 0.8 | 0.52 |
| 15001-15500 | 0.71 | 0.39 | 0.86 | 0.62 | 0.67 | 0.75 | 0.52 |
| 15501-16000 | 0.73 | 0.41 | 0.85 | 0.64 | 0.71 | 0.75 | 0.57 |
| 16001-16500 | 0.73 | 0.42 | 0.86 | 0.64 | 0.68 | 0.79 | 0.53 |
| 16501-17000 | 0.72 | 0.32 | 0.88 | 0.62 | 0.7 | 0.74 | 0.56 |
| 17001-17500 | 0.72 | 0.38 | 0.84 | 0.64 | 0.67 | 0.77 | 0.53 |
| 17501-18000 | 0.69 | 0.33 | 0.86 | 0.57 | 0.71 | 0.72 | 0.54 |
| 18001-18500 | 0.72 | 0.35 | 0.86 | 0.66 | 0.71 | 0.77 | 0.56 |
| 18501-19000 | 0.71 | 0.31 | 0.86 | 0.56 | 0.71 | 0.76 | 0.57 |
| 19001-19500 | 0.67 | 0.33 | 0.86 | 0.49 | 0.73 | 0.73 | 0.57 |
| 19501-20000 | 0.69 | 0.31 | 0.86 | 0.54 | 0.73 | 0.74 | 0.54 |
| 20001-20500 | 0.68 | 0.36 | 0.86 | 0.56 | 0.72 | 0.72 | 0.56 |
| 20501-21000 | 0.71 | 0.34 | 0.85 | 0.59 | 0.73 | 0.77 | 0.59 |
| 21001-21500 | 0.65 | 0.29 | 0.88 | 0.54 | 0.72 | 0.66 | 0.54 |
| 21501-22000 | 0.71 | 0.28 | 0.87 | 0.56 | 0.74 | 0.72 | 0.57 |
| 22001-22500 | 0.69 | 0.27 | 0.86 | 0.51 | 0.74 | 0.71 | 0.56 |
| 22501-23000 | 0.69 | 0.33 | 0.87 | 0.54 | 0.74 | 0.72 | 0.57 |
| 23001-23500 | 0.67 | 0.31 | 0.85 | 0.53 | 0.75 | 0.64 | 0.57 |
| 23501-24000 | 0.68 | 0.31 | 0.86 | 0.51 | 0.72 | 0.68 | 0.61 |
| 24001-24500 | 0.69 | 0.36 | 0.86 | 0.49 | 0.72 | 0.69 | 0.57 |
| 24501-25000 | 0.66 | 0.26 | 0.87 | 0.49 | 0.77 | 0.65 | 0.59 |
| 25001-25500 | 0.65 | 0.26 | 0.87 | 0.44 | 0.75 | 0.62 | 0.58 |
| 25501-26000 | 0.64 | 0.25 | 0.86 | 0.45 | 0.78 | 0.63 | 0.56 |
| 26001-26500 | 0.7 | 0.27 | 0.87 | 0.52 | 0.76 | 0.69 | 0.59 |
| 26501-27000 | 0.65 | 0.26 | 0.86 | 0.49 | 0.74 | 0.66 | 0.58 |
| 27001-27500 | 0.68 | 0.29 | 0.87 | 0.49 | 0.72 | 0.67 | 0.59 |
| 27501-28000 | 0.64 | 0.25 | 0.87 | 0.4 | 0.76 | 0.62 | 0.6 |
| 28001-28500 | 0.65 | 0.25 | 0.87 | 0.43 | 0.76 | 0.61 | 0.59 |
| 28501-29000 | 0.67 | 0.26 | 0.86 | 0.43 | 0.76 | 0.65 | 0.61 |
| 29001-29500 | 0.66 | 0.24 | 0.86 | 0.42 | 0.76 | 0.65 | 0.58 |
| 29501-30000 | 0.66 | 0.27 | 0.87 | 0.47 | 0.74 | 0.67 | 0.55 |
| 30001-30500 | 0.64 | 0.25 | 0.87 | 0.44 | 0.74 | 0.65 | 0.58 |
| 30501-31000 | 0.65 | 0.25 | 0.87 | 0.46 | 0.76 | 0.63 | 0.61 |
| 31001-31500 | 0.64 | 0.26 | 0.88 | 0.43 | 0.77 | 0.62 | 0.59 |
| 31501-32000 | 0.68 | 0.27 | 0.87 | 0.47 | 0.76 | 0.68 | 0.56 |
| 32001-32500 | 0.65 | 0.27 | 0.86 | 0.44 | 0.76 | 0.62 | 0.59 |
| 32501-33000 | 0.66 | 0.28 | 0.86 | 0.48 | 0.76 | 0.67 | 0.58 |
| 33001-33500 | 0.63 | 0.25 | 0.88 | 0.42 | 0.76 | 0.65 | 0.56 |
| 33501-34000 | 0.63 | 0.26 | 0.89 | 0.36 | 0.77 | 0.61 | 0.59 |
| 34001-34500 | 0.61 | 0.24 | 0.88 | 0.38 | 0.77 | 0.58 | 0.6 |
| 34501-35000 | 0.61 | 0.26 | 0.88 | 0.41 | 0.76 | 0.59 | 0.57 |
| 35001-35500 | 0.62 | 0.26 | 0.87 | 0.4 | 0.76 | 0.6 | 0.61 |
| 35501-36000 | 0.62 | 0.26 | 0.86 | 0.4 | 0.74 | 0.61 | 0.57 |
| 36001-36500 | 0.6 | 0.24 | 0.86 | 0.38 | 0.77 | 0.61 | 0.57 |
| 36501-37000 | 0.64 | 0.27 | 0.88 | 0.38 | 0.78 | 0.63 | 0.62 |
| 37001-37500 | 0.63 | 0.26 | 0.87 | 0.39 | 0.77 | 0.62 | 0.6 |
| 37501-38000 | 0.6 | 0.24 | 0.87 | 0.39 | 0.79 | 0.6 | 0.61 |
| 38001-38500 | 0.62 | 0.27 | 0.87 | 0.4 | 0.75 | 0.61 | 0.57 |
| 38501-39000 | 0.63 | 0.26 | 0.87 | 0.42 | 0.74 | 0.61 | 0.57 |
| 39001-39500 | 0.61 | 0.25 | 0.87 | 0.35 | 0.76 | 0.63 | 0.59 |
| 39501-40000 | 0.61 | 0.25 | 0.87 | 0.4 | 0.73 | 0.6 | 0.59 |
| 40001-40500 | 0.61 | 0.25 | 0.87 | 0.35 | 0.76 | 0.62 | 0.59 |
| 40501-40753 | 0.6 | 0.24 | 0.87 | 0.37 | 0.77 | 0.61 | 0.6 |

### Clauses of the Invention

In what follows below an “assay according to the invention” should be taken to mean any assay of the invention defined and described herein, including in the claims, and in particular claims 1 to 7.
1. An assay according to the invention, wherein the set of CpGs comprises at least the CpGs defined by SEQ ID NOs 1 to 500 and identified at nucleotide positions 61 to 62, and wherein the AUC is 0.73.
2. An assay according to the invention, wherein the set of CpGs comprises at least the CpGs defined by SEQ ID NOs 501 to 1000 and identified at nucleotide positions 61 to 62, and wherein the AUC is 0.74.
3. An assay according to the invention, wherein the set of CpGs comprises at least the CpGs defined by SEQ ID NOs 1001 to 1500 and identified at nucleotide positions 61 to 62, and wherein the AUC is 0.75.
4. An assay according to the invention, wherein the set of CpGs comprises at least the CpGs defined by SEQ ID NOs 1501 to 2000 and identified at nucleotide positions 61 to 62, and wherein the AUC is 0.72.
5. An assay according to the invention, wherein the set of CpGs comprises at least the CpGs defined by SEQ ID NOs 2001 to 2500 and identified at nucleotide positions 61 to 62, and wherein the AUC is 0.72.
6. An assay according to the invention, wherein the set of CpGs comprises at least the CpGs defined by SEQ ID NOs 2501 to 3000 and identified at nucleotide positions 61 to 62, and wherein the AUC is 0.69.
7. An assay according to the invention, wherein the set of CpGs comprises at least the CpGs defined by SEQ ID NOs 3001 to 3500 and identified at nucleotide positions 61 to 62, and wherein the AUC is 0.73.
8. An assay according to the invention, wherein the set of CpGs comprises at least the CpGs defined by SEQ ID NOs 3501 to 4000 and identified at nucleotide positions 61 to 62, and wherein the AUC is 0.69.
9. An assay according to the invention, wherein the set of CpGs comprises at least the CpGs defined by SEQ ID NOs 4001 to 4500 and identified at nucleotide positions 61 to 62, and wherein the AUC is 0.73.
10. An assay according to the invention, wherein the set of CpGs comprises at least the CpGs defined by SEQ ID NOs 4501 to 5000 and identified at nucleotide positions 61 to 62, and wherein the AUC is 0.7.
11. An assay according to the invention, wherein the set of CpGs comprises at least the CpGs defined by SEQ ID NOs 5001 to 5500 and identified at nucleotide positions 61 to 62, and wherein the AUC is 0.71.
12. An assay according to the invention, wherein the set of CpGs comprises at least the CpGs defined by SEQ ID NOs 5501 to 6000 and identified at nucleotide positions 61 to 62, and wherein the AUC is 0.7.
13. An assay according to the invention, wherein the set of CpGs comprises at least the CpGs defined by SEQ ID NOs 6001 to 6500 and identified at nucleotide positions 61 to 62, and wherein the AUC is 0.76.
14. An assay according to the invention, wherein the set of CpGs comprises at least the CpGs defined by SEQ ID NOs 6501 to 7000 and identified at nucleotide positions 61 to 62, and wherein the AUC is 0.76.
15. An assay according to the invention, wherein the set of CpGs comprises at least the CpGs defined by SEQ ID NOs 7001 to 7500 and identified at nucleotide positions 61 to 62, and wherein the AUC is 0.66.
16. An assay according to the invention, wherein the set of CpGs comprises at least the CpGs defined by SEQ ID NOs 7501 to 8000 and identified at nucleotide positions 61 to 62, and wherein the AUC is 0.76.
17. An assay according to the invention, wherein the set of CpGs comprises at least the CpGs defined by SEQ ID NOs 8001 to 8500 and identified at nucleotide positions 61 to 62, and wherein the AUC is 0.69.
18. An assay according to the invention, wherein the set of CpGs comprises at least the CpGs defined by SEQ ID NOs 8501 to 9000 and identified at nucleotide positions 61 to 62, and wherein the AUC is 0.73.
19. An assay according to the invention, wherein the set of CpGs comprises at least the CpGs defined by SEQ ID NOs 9001 to 9500 and identified at nucleotide positions 61 to 62, and wherein the AUC is 0.72.
20. An assay according to the invention, wherein the set of CpGs comprises at least the CpGs defined by SEQ ID NOs 9501 to 10000 and identified at nucleotide positions 61 to 62, and wherein the AUC is 0.69.
21. An assay according to the invention, wherein the set of CpGs comprises at least the CpGs defined by SEQ ID NOs 10001 to 10500 and identified at nucleotide positions 61 to 62, and wherein the AUC is 0.69.
22. An assay according to the invention, wherein the set of CpGs comprises at least the CpGs defined by SEQ ID NOs 10501 to 11000 and identified at nucleotide positions 61 to 62, and wherein the AUC is 0.71.
23. An assay according to the invention, wherein the set of CpGs comprises at least the CpGs defined by SEQ ID NOs 11001 to 11500 and identified at nucleotide positions 61 to 62, and wherein the AUC is 0.78.
24. An assay according to the invention, wherein the set of CpGs comprises at least the CpGs defined by SEQ ID NOs 11501 to 12000 and identified at nucleotide positions 61 to 62, and wherein the AUC is 0.67.
25. An assay according to the invention, wherein the set of CpGs comprises at least the CpGs defined by SEQ ID NOs 12001 to 12500 and identified at nucleotide positions 61 to 62, and wherein the AUC is 0.72.
26. An assay according to the invention, wherein the set of CpGs comprises at least the CpGs defined by SEQ ID NOs 12501 to 13000 and identified at nucleotide positions 61 to 62, and wherein the AUC is 0.73.
27. An assay according to the invention, wherein the set of CpGs comprises at least the CpGs defined by SEQ ID NOs 13001 to 13500 and identified at nucleotide positions 61 to 62, and wherein the AUC is 0.73.
28. An assay according to the invention, wherein the set of CpGs comprises at least the CpGs defined by SEQ ID NOs 13501 to 14000 and identified at nucleotide positions 61 to 62, and wherein the AUC is 0.74.
29. An assay according to the invention, wherein the set of CpGs comprises at least the CpGs defined by SEQ ID NOs 14001 to 14500 and identified at nucleotide positions 61 to 62, and wherein the AUC is 0.73.
30. An assay according to the invention, wherein the set of CpGs comprises at least the CpGs defined by SEQ ID NOs 14501 to 15000 and identified at nucleotide positions 61 to 62, and wherein the AUC is 0.72.
31. An assay according to the invention, wherein the set of CpGs comprises at least the CpGs defined by SEQ ID NOs 15001 to 15500 and identified at nucleotide positions 61 to 62, and wherein the AUC is 0.71.
32. An assay according to the invention, wherein the set of CpGs comprises at least the CpGs defined by SEQ ID NOs 15501 to 16000 and identified at nucleotide positions 61 to 62, and wherein the AUC is 0.73.
33. An assay according to the invention, wherein the set of CpGs comprises at least the CpGs defined by SEQ ID NOs 16001 to 16500 and identified at nucleotide positions 61 to 62, and wherein the AUC is 0.73.
34. An assay according to the invention, wherein the set of CpGs comprises at least the CpGs defined by SEQ ID NOs 16501 to 17000 and identified at nucleotide positions 61 to 62, and wherein the AUC is 0.72.
35. An assay according to the invention, wherein the set of CpGs comprises at least the CpGs defined by SEQ ID NOs 17001 to 17500 and identified at nucleotide positions 61 to 62, and wherein the AUC is 0.72.
36. An assay according to the invention, wherein the set of CpGs comprises at least the CpGs defined by SEQ ID NOs 17501 to 18000 and identified at nucleotide positions 61 to 62, and wherein the AUC is 0.69.
37. An assay according to the invention, wherein the set of CpGs comprises at least the CpGs defined by SEQ ID NOs 18001 to 18500 and identified at nucleotide positions 61 to 62, and wherein the AUC is 0.72.
38. An assay according to the invention, wherein the set of CpGs comprises at least the CpGs defined by SEQ ID NOs 18501 to 19000 and identified at nucleotide positions 61 to 62, and wherein the AUC is 0.71.
39. An assay according to the invention, wherein the set of CpGs comprises at least the CpGs defined by SEQ ID NOs 19001 to 19500 and identified at nucleotide positions 61 to 62, and wherein the AUC is 0.67.
40. An assay according to the invention, wherein the set of CpGs comprises at least the CpGs defined by SEQ ID NOs 19501 to 20000 and identified at nucleotide positions 61 to 62, and wherein the AUC is 0.69.
41. An assay according to the invention, wherein the set of CpGs comprises at least the CpGs defined by SEQ ID NOs 20001 to 20500 and identified at nucleotide positions 61 to 62, and wherein the AUC is 0.68.
42. An assay according to the invention, wherein the set of CpGs comprises at least the CpGs defined by SEQ ID NOs 20501 to 21000 and identified at nucleotide positions 61 to 62, and wherein the AUC is 0.71
43. An assay according to the invention, wherein the set of CpGs comprises at least the CpGs defined by SEQ ID NOs 21001 to 21500 and identified at nucleotide positions 61 to 62, and wherein the AUC is 0.71.
44. An assay according to the invention, wherein the set of CpGs comprises at least the CpGs defined by SEQ ID NOs 21501 to 22000 and identified at nucleotide positions 61 to 62, and wherein the AUC is 0.71.
45. An assay according to the invention, wherein the set of CpGs comprises at least the CpGs defined by SEQ ID NOs 22001 to 22500 and identified at nucleotide positions 61 to 62, and wherein the AUC is 0.69.
46. An assay according to the invention, wherein the set of CpGs comprises at least the CpGs defined by SEQ ID NOs 22501 to 23000 and identified at nucleotide positions 61 to 62, and wherein the AUC is 0.69.
47. An assay according to the invention, wherein the set of CpGs comprises at least the CpGs defined by SEQ ID NOs 23001 to 23500 and identified at nucleotide positions 61 to 62, and wherein the AUC is 0.67.
48. An assay according to the invention, wherein the set of CpGs comprises at least the CpGs defined by SEQ ID NOs 23501 to 24000 and identified at nucleotide positions 61 to 62, and wherein the AUC is 0.68.
49. An assay according to the invention, wherein the set of CpGs comprises at least the CpGs defined by SEQ ID NOs 24001 to 24500 and identified at nucleotide positions 61 to 62, and wherein the AUC is 0.69.
50. An assay according to the invention, wherein the set of CpGs comprises at least the CpGs defined by SEQ ID NOs 24501 to 25000 and identified at nucleotide positions 61 to 62, and wherein the AUC is 0.66.
51. An assay according to the invention, wherein the set of CpGs comprises at least the CpGs defined by SEQ ID NOs 25001 to 25500 and identified at nucleotide positions 61 to 62, and wherein the AUC is 0.65.
52. An assay according to the invention, wherein the set of CpGs comprises at least the CpGs defined by SEQ ID NOs 25501 to 26000 and identified at nucleotide positions 61 to 62, and wherein the AUC is 0.64.
53. An assay according to the invention, wherein the set of CpGs comprises at least the CpGs defined by SEQ ID NOs 26001 to 26500 and identified at nucleotide positions 61 to 62, and wherein the AUC is 0.7.
54. An assay according to the invention, wherein the set of CpGs comprises at least the CpGs defined by SEQ ID NOs 26501 to 27000 and identified at nucleotide positions 61 to 62, and wherein the AUC is 0.65.
55. An assay according to the invention, wherein the set of CpGs comprises at least the CpGs defined by SEQ ID NOs 27001 to 27500 and identified at nucleotide positions 61 to 62, and wherein the AUC is 0.68.
56. An assay according to the invention, wherein the set of CpGs comprises at least the CpGs defined by SEQ ID NOs 27501 to 28000 and identified at nucleotide positions 61 to 62, and wherein the AUC is 0.64.
57. An assay according to the invention, wherein the set of CpGs comprises at least the CpGs defined by SEQ ID NOs 28001 to 28500 and identified at nucleotide positions 61 to 62, and wherein the AUC is 0.65.
58. An assay according to the invention, wherein the set of CpGs comprises at least the CpGs defined by SEQ ID NOs 28501 to 29000 and identified at nucleotide positions 61 to 62, and wherein the AUC is 0.67.
59. An assay according to the invention, wherein the set of CpGs comprises at least the CpGs defined by SEQ ID NOs 29001 to 29500 and identified at nucleotide positions 61 to 62, and wherein the AUC is 0.66.
60. An assay according to the invention, wherein the set of CpGs comprises at least the CpGs defined by SEQ ID NOs 29501 to 30000 and identified at nucleotide positions 61 to 62, and wherein the AUC is 0.66.
61. An assay according to the invention, wherein the set of CpGs comprises at least the CpGs defined by SEQ ID NOs 30001 to 30500 and identified at nucleotide positions 61 to 62, and wherein the AUC is 0.64.
62. An assay according to the invention, wherein the set of CpGs comprises at least the CpGs defined by SEQ ID NOs 30501 to 31000 and identified at nucleotide positions 61 to 62, and wherein the AUC is 0.65.
63. An assay according to the invention, wherein the set of CpGs comprises at least the CpGs defined by SEQ ID NOs 31001 to 31500 and identified at nucleotide positions 61 to 62, and wherein the AUC is 0.64.
64. An assay according to the invention, wherein the set of CpGs comprises at least the CpGs defined by SEQ ID NOs 31501 to 32000 and identified at nucleotide positions 61 to 62, and wherein the AUC is 0.68.
65. An assay according to the invention, wherein the set of CpGs comprises at least the CpGs defined by SEQ ID NOs 32001 to 32500 and identified at nucleotide positions 61 to 62, and wherein the AUC is 0.65.
66. An assay according to the invention, wherein the set of CpGs comprises at least the CpGs defined by SEQ ID NOs 32501 to 33000 and identified at nucleotide positions 61 to 62, and wherein the AUC is 0.66.
67. An assay according to the invention, wherein the set of CpGs comprises at least the CpGs defined by SEQ ID NOs 33001 to 33500 and identified at nucleotide positions 61 to 62, and wherein the AUC is 0.63.
68. An assay according to the invention, wherein the set of CpGs comprises at least the CpGs defined by SEQ ID NOs 33501 to 34000 and identified at nucleotide positions 61 to 62, and wherein the AUC is 0.63.
69. An assay according to the invention, wherein the set of CpGs comprises at least the CpGs defined by SEQ ID NOs 34001 to 34500 and identified at nucleotide positions 61 to 62, and wherein the AUC is 0.61.
70. An assay according to the invention, wherein the set of CpGs comprises at least the CpGs defined by SEQ ID NOs 34501 to 35000 and identified at nucleotide positions 61 to 62, and wherein the AUC is 0.61.
71. An assay according to the invention, wherein the set of CpGs comprises at least the CpGs defined by SEQ ID NOs 35001 to 35500 and identified at nucleotide positions 61 to 62, and wherein the AUC is 0.62.
72. An assay according to the invention, wherein the set of CpGs comprises at least the CpGs defined by SEQ ID NOs 35501 to 36000 and identified at nucleotide positions 61 to 62, and wherein the AUC is 0.62.
73. An assay according to the invention, wherein the set of CpGs comprises at least the CpGs defined by SEQ ID NOs 36001 to 36500 and identified at nucleotide positions 61 to 62, and wherein the AUC is 0.6.
74. An assay according to the invention, wherein the set of CpGs comprises at least the CpGs defined by SEQ ID NOs 36501 to 37000 and identified at nucleotide positions 61 to 62, and wherein the AUC is 0.64.
75. An assay according to the invention, wherein the set of CpGs comprises at least the CpGs defined by SEQ ID NOs 37001 to 37500 and identified at nucleotide positions 61 to 62, and wherein the AUC is 0.63.
76. An assay according to the invention, wherein the set of CpGs comprises at least the CpGs defined by SEQ ID NOs 37501 to 38000 and identified at nucleotide positions 61 to 62, and wherein the AUC is 0.6.
77. An assay according to the invention, wherein the set of CpGs comprises at least the CpGs defined by SEQ ID NOs 38001 to 38500 and identified at nucleotide positions 61 to 62, and wherein the AUC is 0.62.
78. An assay according to the invention, wherein the set of CpGs comprises at least the CpGs defined by SEQ ID NOs 38501 to 39000 and identified at nucleotide positions 61 to 62, and wherein the AUC is 0.63.
79. An assay according to the invention, wherein the set of CpGs comprises at least the CpGs defined by SEQ ID NOs 39001 to 39500 and identified at nucleotide positions 61 to 62, and wherein the AUC is 0.61.
80. An assay according to the invention, wherein the set of CpGs comprises at least the CpGs defined by SEQ ID NOs 39501 to 40000 and identified at nucleotide positions 61 to 62, and wherein the AUC is 0.61.
81. An assay according to the invention, wherein the set of CpGs comprises at least the CpGs defined by SEQ ID NOs 40001 to 40500 and identified at nucleotide positions 61 to 62, and wherein the AUC is 0.61.
82. An assay according to the invention, wherein the set of CpGs comprises at least the CpGs defined by SEQ ID NOs 40501 to 40753 and identified at nucleotide positions 61 to 62, and wherein the AUC is 0.6.
83. An assay according to the invention, wherein the set of CpGs comprises at least the CpGs defined by SEQ ID NOs 500 to 40753 and identified at nucleotide positions 61 to 62, and wherein the AUC is 0.84.
84. An assay according to the invention, wherein the set of CpGs comprises at least the CpGs defined by SEQ ID NOs 1000 to 40753 and identified at nucleotide positions 61 to 62, and wherein the AUC is 0.84.
85. An assay according to the invention, wherein the set of CpGs comprises at least the CpGs defined by SEQ ID NOs 1500 to 40753 and identified at nucleotide positions 61 to 62, and wherein the AUC is 0.81.
86. An assay according to the invention, wherein the set of CpGs comprises at least the CpGs defined by SEQ ID NOs 2000 to 40753 and identified at nucleotide positions 61 to 62, and wherein the AUC is 0.81.
87. An assay according to the invention, wherein the set of CpGs comprises at least the CpGs defined by SEQ ID NOs 2500 to 40753 and identified at nucleotide positions 61 to 62, and wherein the AUC is 0.8.
88. An assay according to the invention, wherein the set of CpGs comprises at least the CpGs defined by SEQ ID NOs 3000 to 40753 and identified at nucleotide positions 61 to 62, and wherein the AUC is 0.8.
89. An assay according to the invention, wherein the set of CpGs comprises at least the CpGs defined by SEQ ID NOs 3500 to 40753 and identified at nucleotide positions 61 to 62, and wherein the AUC is 0.79.
90. An assay according to the invention, wherein the set of CpGs comprises at least the CpGs defined by SEQ ID NOs 4000 to 40753 and identified at nucleotide positions 61 to 62, and wherein the AUC is 0.78.
91. An assay according to the invention, wherein the set of CpGs comprises at least the CpGs defined by SEQ ID NOs 4500 to 40753 and identified at nucleotide positions 61 to 62, and wherein the AUC is 0.78.
92. An assay according to the invention, wherein the set of CpGs comprises at least the CpGs defined by SEQ ID NOs 5000 to 40753 and identified at nucleotide positions 61 to 62, and wherein the AUC is 0.77.
93. An assay according to the invention, wherein the set of CpGs comprises at least the CpGs defined by SEQ ID NOs 5500 to 40753 and identified at nucleotide positions 61 to 62, and wherein the AUC is 0.76.
94. An assay according to the invention, wherein the set of CpGs comprises at least the CpGs defined by SEQ ID NOs 6000 to 40753 and identified at nucleotide positions 61 to 62, and wherein the AUC is 0.74.
95. An assay according to the invention, wherein the set of CpGs comprises at least the CpGs defined by SEQ ID NOs 6500 to 40753 and identified at nucleotide positions 61 to 62, and wherein the AUC is 0.74.
96. An assay according to the invention, wherein the set of CpGs comprises at least the CpGs defined by SEQ ID NOs 7000 to 40753 and identified at nucleotide positions 61 to 62, and wherein the AUC is 0.74.
97. An assay according to the invention, wherein the set of CpGs comprises at least the CpGs defined by SEQ ID NOs 7500 to 40753 and identified at nucleotide positions 61 to 62, and wherein the AUC is 0.74.
98. An assay according to the invention, wherein the set of CpGs comprises at least the CpGs defined by SEQ ID NOs 8000 to 40753 and identified at nucleotide positions 61 to 62, and wherein the AUC is 0.73.
99. An assay according to the invention, wherein the set of CpGs comprises at least the CpGs defined by SEQ ID NOs 8500 to 40753 and identified at nucleotide positions 61 to 62, and wherein the AUC is 0.73.
100. An assay according to the invention, wherein the set of CpGs comprises at least the CpGs defined by SEQ ID NOs 9000 to 40753 and identified at nucleotide positions 61 to 62, and wherein the AUC is 0.72.
101. An assay according to the invention, wherein the set of CpGs comprises at least the CpGs defined by SEQ ID NOs 9500 to 40753 and identified at nucleotide positions 61 to 62, and wherein the AUC is 0.72.
102. An assay according to the invention, wherein the set of CpGs comprises at least the CpGs defined by SEQ ID NOs 10000 to 40753 and identified at nucleotide positions 61 to 62, and wherein the AUC is 0.72.
103. An assay according to the invention, wherein the set of CpGs comprises at least the CpGs defined by SEQ ID NOs 11000 to 40753 and identified at nucleotide positions 61 to 62, and wherein the AUC is 0.71.
104. An assay according to the invention, wherein the set of CpGs comprises at least the CpGs defined by SEQ ID NOs 12000 to 40753 and identified at nucleotide positions 61 to 62, and wherein the AUC is 0.71.
105. An assay according to the invention, wherein the set of CpGs comprises at least the CpGs defined by SEQ ID NOs 13000 to 40753 and identified at nucleotide positions 61 to 62, and wherein the AUC is 0.71.
106. An assay according to the invention, wherein the set of CpGs comprises at least the CpGs defined by SEQ ID NOs 14000 to 40753 and identified at nucleotide positions 61 to 62, and wherein the AUC is 0.68.
107. An assay according to the invention, wherein the set of CpGs comprises at least the CpGs defined by SEQ ID NOs 15000 to 40753 and identified at nucleotide positions 61 to 62, and wherein the AUC is 0.69.
108. An assay according to the invention, wherein the set of CpGs comprises at least the CpGs defined by SEQ ID NOs 16000 to 40753 and identified at nucleotide positions 61 to 62, and wherein the AUC is 0.68.
109. An assay according to the invention, wherein the set of CpGs comprises at least the CpGs defined by SEQ ID NOs 17000 to 40753 and identified at nucleotide positions 61 to 62, and wherein the AUC is 0.67.
110. An assay according to the invention, wherein the set of CpGs comprises at least the CpGs defined by SEQ ID NOs 18000 to 40753 and identified at nucleotide positions 61 to 62, and wherein the AUC is 0.67.
111. An assay according to the invention, wherein the set of CpGs comprises at least the CpGs defined by SEQ ID NOs 19000 to 40753 and identified at nucleotide positions 61 to 62, and wherein the AUC is 0.66.
112. An assay according to the invention, wherein the set of CpGs comprises at least the CpGs defined by SEQ ID NOs 20000 to 40753 and identified at nucleotide positions 61 to 62, and wherein the AUC is 0.66.
113. An assay according to the invention, wherein the set of CpGs comprises at least the CpGs defined by SEQ ID NOs 22000 to 40753 and identified at nucleotide positions 61 to 62, and wherein the AUC is 0.67.
114. An assay according to the invention, wherein the set of CpGs comprises at least the CpGs defined by SEQ ID NOs 24000 to 40753 and identified at nucleotide positions 61 to 62, and wherein the AUC is 0.67.
115. An assay according to the invention, wherein the set of CpGs comprises at least the CpGs defined by SEQ ID NOs 26000 to 40753 and identified at nucleotide positions 61 to 62, and wherein the AUC is 0.67.
116. An assay according to the invention, wherein the set of CpGs comprises at least the CpGs defined by SEQ ID NOs 28000 to 40753 and identified at nucleotide positions 61 to 62, and wherein the AUC is 0.64.
117. An assay according to the invention, wherein the set of CpGs comprises at least the CpGs defined by SEQ ID NOs 30000 to 40753 and identified at nucleotide positions 61 to 62, and wherein the AUC is 0.63.
118. An assay according to the invention, wherein the set of CpGs comprises at least the CpGs defined by SEQ ID NOs 32000 to 40753 and identified at nucleotide positions 61 to 62, and wherein the AUC is 0.62.
119. An assay according to the invention, wherein the set of CpGs comprises at least the CpGs defined by SEQ ID NOs 34000 to 40753 and identified at nucleotide positions 61 to 62, and wherein the AUC is 0.62.
120. An assay according to the invention, wherein the set of CpGs comprises at least the CpGs defined by SEQ ID NOs 36000 to 40753 and identified at nucleotide positions 61 to 62, and wherein the AUC is 0.61.
121. An assay according to the invention, wherein the set of CpGs comprises at least the CpGs defined by SEQ ID NOs 38000 to 40753 and identified at nucleotide positions 61 to 62, and wherein the AUC is 0.61.
122. An assay according to the invention, wherein the set of CpGs comprises at least the CpGs defined by SEQ ID NOs 1 to 500 and identified at nucleotide positions 61 to 62, and wherein the AUC is 0.73.
123. An assay according to the invention, wherein the set of CpGs comprises at least the CpGs defined by SEQ ID NOs 1 to 1000 and identified at nucleotide positions 61 to 62, and wherein the AUC is 0.77.
124. An assay according to the invention, wherein the set of CpGs comprises at least the CpGs defined by SEQ ID NOs 1 to 1500 and identified at nucleotide positions 61 to 62, and wherein the AUC is 0.80.
125. An assay according to the invention, wherein the set of CpGs comprises at least the CpGs defined by SEQ ID NOs 1 to 2000 and identified at nucleotide positions 61 to 62, and wherein the AUC is 0.81.
126. An assay according to the invention, wherein the set of CpGs comprises at least the CpGs defined by SEQ ID NOs 1 to 2500 and identified at nucleotide positions 61 to 62, and wherein the AUC is 0.82.
127. An assay according to the invention, wherein the set of CpGs comprises at least the CpGs defined by SEQ ID NOs 1 to 3000 and identified at nucleotide positions 61 to 62, and wherein the AUC is 0.82.
128. An assay according to the invention, wherein the set of CpGs comprises at least the CpGs defined by SEQ ID NOs 1 to 3500 and identified at nucleotide positions 61 to 62, and wherein the AUC is 0.83.
129. An assay according to the invention, wherein the set of CpGs comprises at least the CpGs defined by SEQ ID NOs 1 to 4000 and identified at nucleotide positions 61 to 62, and wherein the AUC is 0.83.
130. An assay according to the invention, wherein the set of CpGs comprises at least the CpGs defined by SEQ ID NOs 1 to 4500 and identified at nucleotide positions 61 to 62, and wherein the AUC is 0.83.
131. An assay according to the invention, wherein the set of CpGs comprises at least the CpGs defined by SEQ ID NOs 1 to 5000 and identified at nucleotide positions 61 to 62, and wherein the AUC is 0.84.
132. An assay according to the invention, wherein the set of CpGs comprises at least the CpGs defined by SEQ ID NOs 1 to 5500 and identified at nucleotide positions 61 to 62, and wherein the AUC is 0.83.
133. An assay according to the invention, wherein the set of CpGs comprises at least the CpGs defined by SEQ ID NOs 1 to 6000 and identified at nucleotide positions 61 to 62, and wherein the AUC is 0.83.
134. An assay according to the invention, wherein the set of CpGs comprises at least the CpGs defined by SEQ ID NOs 1 to 6500 and identified at nucleotide positions 61 to 62, and wherein the AUC is 0.84.
135. An assay according to the invention, wherein the set of CpGs comprises at least the CpGs defined by SEQ ID NOs 1 to 7000 and identified at nucleotide positions 61 to 62, and wherein the AUC is 0.84.
136. An assay according to the invention, wherein the set of CpGs comprises at least the CpGs defined by SEQ ID NOs 1 to 7500 and identified at nucleotide positions 61 to 62, and wherein the AUC is 0.84.
137. An assay according to the invention, wherein the set of CpGs comprises at least the CpGs defined by SEQ ID NOs 1 to 8000 and identified at nucleotide positions 61 to 62, and wherein the AUC is 0.84.
138. An assay according to the invention, wherein the set of CpGs comprises at least the CpGs defined by SEQ ID NOs 1 to 8500 and identified at nucleotide positions 61 to 62, and wherein the AUC is 0.84.
139. An assay according to the invention, wherein the set of CpGs comprises at least the CpGs defined by SEQ ID NOs 1 to 9000 and identified at nucleotide positions 61 to 62, and wherein the AUC is 0.84.
140. An assay according to the invention, wherein the set of CpGs comprises at least the CpGs defined by SEQ ID NOs 1 to 9500 and identified at nucleotide positions 61 to 62, and wherein the AUC is 0.84.
141. An assay according to the invention, wherein the set of CpGs comprises at least the CpGs defined by SEQ ID NOs 1 to 10000 and identified at nucleotide positions 61 to 62, and wherein the AUC is 0.84.
142. An assay according to the invention, wherein the set of CpGs comprises at least the CpGs defined by SEQ ID NOs 1 to 11000 and identified at nucleotide positions 61 to 62, and wherein the AUC is 0.84.
143. An assay according to the invention, wherein the set of CpGs comprises at least the CpGs defined by SEQ ID NOs 1 to 12000 and identified at nucleotide positions 61 to 62, and wherein the AUC is 0.84.
144. An assay according to the invention, wherein the set of CpGs comprises at least the CpGs defined by SEQ ID NOs 1 to 13000 and identified at nucleotide positions 61 to 62, and wherein the AUC is 0.84.
145. An assay according to the invention, wherein the set of CpGs comprises at least the CpGs defined by SEQ ID NOs 1 to 14000 and identified at nucleotide positions 61 to 62, and wherein the AUC is 0.84.
146. An assay according to the invention, wherein the set of CpGs comprises at least the CpGs defined by SEQ ID NOs 1 to 15000 and identified at nucleotide positions 61 to 62, and wherein the AUC is 0.84.
147. An assay according to the invention, wherein the set of CpGs comprises at least the CpGs defined by SEQ ID NOs 1 to 16000 and identified at nucleotide positions 61 to 62, and wherein the AUC is 0.84.
148. An assay according to the invention, wherein the set of CpGs comprises at least the CpGs defined by SEQ ID NOs 1 to 17000 and identified at nucleotide positions 61 to 62, and wherein the AUC is 0.85.
149. An assay according to the invention, wherein the set of CpGs comprises at least the CpGs defined by SEQ ID NOs 1 to 18000 and identified at nucleotide positions 61 to 62, and wherein the AUC is 0.85.
150. An assay according to the invention, wherein the set of CpGs comprises at least the CpGs defined by SEQ ID NOs 1 to 19000 and identified at nucleotide positions 61 to 62, and wherein the AUC is 0.85.
151. An assay according to the invention, wherein the set of CpGs comprises at least the CpGs defined by SEQ ID NOs 1 to 20000 and identified at nucleotide positions 61 to 62, and wherein the AUC is 0.85.
152. An assay according to the invention, wherein the set of CpGs comprises at least the CpGs defined by SEQ ID NOs 1 to 22000 and identified at nucleotide positions 61 to 62, and wherein the AUC is 0.85.
153. An assay according to the invention, wherein the set of CpGs comprises at least the CpGs defined by SEQ ID NOs 1 to 24000 and identified at nucleotide positions 61 to 62, and wherein the AUC is 0.85.
154. An assay according to the invention, wherein the set of CpGs comprises at least the CpGs defined by SEQ ID NOs 1 to 26000 and identified at nucleotide positions 61 to 62, and wherein the AUC is 0.85.
155. An assay according to the invention, wherein the set of CpGs comprises at least the CpGs defined by SEQ ID NOs 1 to 28000 and identified at nucleotide positions 61 to 62, and wherein the AUC is 0.85.
156. An assay according to the invention, wherein the set of CpGs comprises at least the CpGs defined by SEQ ID NOs 1 to 30000 and identified at nucleotide positions 61 to 62, and wherein the AUC is 0.85.
157. An assay according to the invention, wherein the set of CpGs comprises at least the CpGs defined by SEQ ID NOs 1 to 32000 and identified at nucleotide positions 61 to 62, and wherein the AUC is 0.85.
158. An assay according to the invention, wherein the set of CpGs comprises at least the CpGs defined by SEQ ID NOs 1 to 34000 and identified at nucleotide positions 61 to 62, and wherein the AUC is 0.85.
159. An assay according to the invention, wherein the set of CpGs comprises at least the CpGs defined by SEQ ID NOs 1 to 36000 and identified at nucleotide positions 61 to 62, and wherein the AUC is 0.85.
160. An assay according to the invention, wherein the set of CpGs comprises at least the CpGs defined by SEQ ID NOs 1 to 38000 and identified at nucleotide positions 61 to 62, and wherein the AUC is 0.85.
161. An assay according to the invention, wherein the set of CpGs comprises at least the CpGs defined by SEQ ID NOs 1 to 40753 and identified at nucleotide positions 61 to 62, and wherein the AUC is 0.85.
   In what follows below an “assay according to the invention” should be taken to mean any assay of the invention defined and described herein, including in the claims, and in particular claims 1 to 7, and more particular claim 17.
   In any of the below described assays when a breast cancer index value threshold of −0.235 is being applied, when the WID-BC-index for the individual is about −0.235 or more, the individual may be classified as harbouring breast cancer, wherein when the WID-BC index for the individual is less than about −0.235 the individual may be classified as not harbouring breast cancer, subject to the specified sensitivity and specificity of the assay.
   In any of the below described assays when a breast cancer index value threshold of 0.090 is being applied, when the WID-BC-index for the individual is about 0.090 or more, the individual may be classified as harbouring breast cancer, wherein when the WID-BC index for the individual is less than about 0.090 the individual may be classified as not harbouring breast cancer, subject to the specified sensitivity and specificity of the assay.
   In any of the below described assays when a breast cancer index value threshold of 0.587 is being applied, when the WID-BC-index for the individual is about 0.587 or more, the individual may be classified as harbouring breast cancer, wherein when the WID-BC index for the individual is less than about 0.587 the individual may be classified as not harbouring breast cancer, subject to the specified sensitivity and specificity of the assay.
162. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 500 and identified at nucleotide positions 61 to 62, and wherein the sensitivity is at least 85% and specificity is at least 52%.
163. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 1000 and identified at nucleotide positions 61 to 62, and wherein the sensitivity is at least 88% and specificity is at least 49%.
164. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 1500 and identified at nucleotide positions 61 to 62, and wherein the sensitivity is at least 92% and specificity is at least 51%.
165. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 2000 and identified at nucleotide positions 61 to 62, and wherein the sensitivity is at least 94% and specificity is at least 51%.
166. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 2500 and identified at nucleotide positions 61 to 62, and wherein the sensitivity is at least 93% and specificity is at least 51%.
167. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 3000 and identified at nucleotide positions 61 to 62, and wherein the sensitivity is at least 93% and specificity is at least 51%.
168. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 3500 and identified at nucleotide positions 61 to 62, and wherein the sensitivity is at least 94% and specificity is at least 49%.
169. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 4000 and identified at nucleotide positions 61 to 62, and wherein the sensitivity is at least 95% and specificity is at least 47%.
170. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 4500 and identified at nucleotide positions 61 to 62, and wherein the sensitivity is at least 94% and specificity is at least 48%.
171. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 5000 and identified at nucleotide positions 61 to 62, and wherein the sensitivity is at least 96% and specificity is at least 49%.
172. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 5500 and identified at nucleotide positions 61 to 62, and wherein the sensitivity is at least 94% and specificity is at least 49%.
173. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 6000 and identified at nucleotide positions 61 to 62, and wherein the sensitivity is at least 95% and specificity is at least 50%.
174. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 6500 and identified at nucleotide positions 61 to 62, and wherein the sensitivity is at least 95% and specificity is at least 49%.
175. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 7000 and identified at nucleotide positions 61 to 62, and wherein the sensitivity is at least 95% and specificity is at least 50%.
176. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 7500 and identified at nucleotide positions 61 to 62, and wherein the sensitivity is at least 95% and specificity is at least 52%.
177. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 8000 and identified at nucleotide positions 61 to 62, and wherein the sensitivity is at least 95% and specificity is at least 51%.
178. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 8500 and identified at nucleotide positions 61 to 62, and wherein the sensitivity is at least 94% and specificity is at least 53%.
179. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 9000 and identified at nucleotide positions 61 to 62, and wherein the sensitivity is at least 95% and specificity is at least 52%.
180. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 9500 and identified at nucleotide positions 61 to 62, and wherein the sensitivity is at least 95% and specificity is at least 50%.
181. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 10000 and identified at nucleotide positions 61 to 62, and wherein the sensitivity is at least 95% and specificity is at least 50%.
182. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 11000 and identified at nucleotide positions 61 to 62, and wherein the sensitivity is at least 95% and specificity is at least 51%.
183. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 12000 and identified at nucleotide positions 61 to 62, and wherein the sensitivity is at least 95% and specificity is at least 52%.
184. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 13000 and identified at nucleotide positions 61 to 62, and wherein the sensitivity is at least 95% and specificity is at least 52%.
185. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 14000 and identified at nucleotide positions 61 to 62, and wherein the sensitivity is at least 95% and specificity is at least 52%.
186. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 15000 and identified at nucleotide positions 61 to 62, and wherein the sensitivity is at least 95% and specificity is at least 52%.
187. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 16000 and identified at nucleotide positions 61 to 62, and wherein the sensitivity is at least 95% and specificity is at least 53%.
188. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 17000 and identified at nucleotide positions 61 to 62, and wherein the sensitivity is at least 95% and specificity is at least 51%.
189. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 18000 and identified at nucleotide positions 61 to 62, and wherein the sensitivity is at least 95% and specificity is at least 51%.
190. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 19000 and identified at nucleotide positions 61 to 62, and wherein the sensitivity is at least 94% and specificity is at least 51%.
191. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 20000 and identified at nucleotide positions 61 to 62, and wherein the sensitivity is at least 94% and specificity is at least 51%.
192. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 22000 and identified at nucleotide positions 61 to 62, and wherein the sensitivity is at least 94% and specificity is at least 51%.
193. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 24000 and identified at nucleotide positions 61 to 62, and wherein the sensitivity is at least 94% and specificity is at least 51%.
194. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 26000 and identified at nucleotide positions 61 to 62, and wherein the sensitivity is at least 94% and specificity is at least 51%.
195. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 28000 and identified at nucleotide positions 61 to 62, and wherein the sensitivity is at least 94% and specificity is at least 51%.
196. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 30000 and identified at nucleotide positions 61 to 62, and wherein the sensitivity is at least 94% and specificity is at least 51%.
197. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 32000 and identified at nucleotide positions 61 to 62, and wherein the sensitivity is at least 94% and specificity is at least 50%.
198. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 34000 and identified at nucleotide positions 61 to 62, and wherein the sensitivity is at least 94% and specificity is at least 50%.
199. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 36000 and identified at nucleotide positions 61 to 62, and wherein the sensitivity is at least 94% and specificity is at least 50%.
200. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 38000 and identified at nucleotide positions 61 to 62, and wherein the sensitivity is at least 94% and specificity is at least 50%.
201. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 40753 and identified at nucleotide positions 61 to 62, and wherein the sensitivity is at least 94% and specificity is at least 50%.
202. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 500 and identified at nucleotide positions 61 to 62, and wherein the sensitivity is at least 63% and specificity is at least 69%.
203. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 1000 and identified at nucleotide positions 61 to 62, and wherein the sensitivity is at least 68% and specificity is at least 73%.
204. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 1500 and identified at nucleotide positions 61 to 62, and wherein the sensitivity is at least 73% and specificity is at least 75%.
205. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 2000 and identified at nucleotide positions 61 to 62, and wherein the sensitivity is at least 69% and specificity is at least 78%.
206. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 2500 and identified at nucleotide positions 61 to 62, and wherein the sensitivity is at least 68% and specificity is at least 78%.
207. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 3000 and identified at nucleotide positions 61 to 62, and wherein the sensitivity is at least 71% and specificity is at least 78%.
208. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 3500 and identified at nucleotide positions 61 to 62, and wherein the sensitivity is at least 72% and specificity is at least 79%.
209. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 4000 and identified at nucleotide positions 61 to 62, and wherein the sensitivity is at least 69% and specificity is at least 80%.
210. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 4500 and identified at nucleotide positions 61 to 62, and wherein the sensitivity is at least 72% and specificity is at least 79%.
211. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 5000 and identified at nucleotide positions 61 to 62, and wherein the sensitivity is at least 72% and specificity is at least 80%.
212. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 5500 and identified at nucleotide positions 61 to 62, and wherein the sensitivity is at least 73% and specificity is at least 79%.
213. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 6000 and identified at nucleotide positions 61 to 62, and wherein the sensitivity is at least 73% and specificity is at least 79%.
214. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 6500 and identified at nucleotide positions 61 to 62, and wherein the sensitivity is at least 71% and specificity is at least 79%.
215. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 7000 and identified at nucleotide positions 61 to 62, and wherein the sensitivity is at least 73% and specificity is at least 80%.
216. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 7500 and identified at nucleotide positions 61 to 62, and wherein the sensitivity is at least 72% and specificity is at least 80%.
217. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 8000 and identified at nucleotide positions 61 to 62, and wherein the sensitivity is at least 74% and specificity is at least 79%.
218. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 8500 and identified at nucleotide positions 61 to 62, and wherein the sensitivity is at least 73% and specificity is at least 80%.
219. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 9000 and identified at nucleotide positions 61 to 62, and wherein the sensitivity is at least 75% and specificity is at least 79%.
220. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 9500 and identified at nucleotide positions 61 to 62, and wherein the sensitivity is at least 74% and specificity is at least 78%.
221. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 10000 and identified at nucleotide positions 61 to 62, and wherein the sensitivity is at least 74% and specificity is at least 80%.
222. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 11000 and identified at nucleotide positions 61 to 62, and wherein the sensitivity is at least 73% and specificity is at least 80%.
223. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 12000 and identified at nucleotide positions 61 to 62, and wherein the sensitivity is at least 74% and specificity is at least 80%.
224. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 13000 and identified at nucleotide positions 61 to 62, and wherein the sensitivity is at least 74% and specificity is at least 80%.
225. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 14000 and identified at nucleotide positions 61 to 62, and wherein the sensitivity is at least 76% and specificity is at least 80%.
226. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 15000 and identified at nucleotide positions 61 to 62, and wherein the sensitivity is at least 74% and specificity is at least 80%.
227. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 16000 and identified at nucleotide positions 61 to 62, and wherein the sensitivity is at least 74% and specificity is at least 80%.
228. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 17000 and identified at nucleotide positions 61 to 62, and wherein the sensitivity is at least 74% and specificity is at least 80%.
229. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 18000 and identified at nucleotide positions 61 to 62, and wherein the sensitivity is at least 76% and specificity is at least 80%.
230. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 19000 and identified at nucleotide positions 61 to 62, and wherein the sensitivity is at least 77% and specificity is at least 80%.
231. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 20000 and identified at nucleotide positions 61 to 62, and wherein the sensitivity is at least 76% and specificity is at least 80%.
232. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 22000 and identified at nucleotide positions 61 to 62, and wherein the sensitivity is at least 77% and specificity is at least 80%.
233. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 24000 and identified at nucleotide positions 61 to 62, and wherein the sensitivity is at least 77% and specificity is at least 80%.
234. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 26000 and identified at nucleotide positions 61 to 62, and wherein the sensitivity is at least 77% and specificity is at least 80%.
235. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 28000 and identified at nucleotide positions 61 to 62, and wherein the sensitivity is at least 77% and specificity is at least 80%.
236. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 30000 and identified at nucleotide positions 61 to 62, and wherein the sensitivity is at least 77% and specificity is at least 80%.
237. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 32000 and identified at nucleotide positions 61 to 62, and wherein the sensitivity is at least 77% and specificity is at least 80%.
238. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 34000 and identified at nucleotide positions 61 to 62, and wherein the sensitivity is at least 78% and specificity is at least 80%.
239. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 36000 and identified at nucleotide positions 61 to 62, and wherein the sensitivity is at least 78% and specificity is at least 80%.
240. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 38000 and identified at nucleotide positions 61 to 62, and wherein the sensitivity is at least 77% and specificity is at least 80%.
241. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 40753 and identified at nucleotide positions 61 to 62, and wherein the sensitivity is at least 78% and specificity is at least 80%.
242. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 500 and identified at nucleotide positions 61 to 62, and wherein the sensitivity is at least 26% and specificity is at least 93%.
243. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 1000 and identified at nucleotide positions 61 to 62, and wherein the sensitivity is at least 29% and specificity is at least 95%.
244. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 1500 and identified at nucleotide positions 61 to 62, and wherein the sensitivity is at least 29% and specificity is at least 94%.
245. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 2000 and identified at nucleotide positions 61 to 62, and wherein the sensitivity is at least 33% and specificity is at least 94%.
246. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 2500 and identified at nucleotide positions 61 to 62, and wherein the sensitivity is at least 31% and specificity is at least 95%.
247. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 3000 and identified at nucleotide positions 61 to 62, and wherein the sensitivity is at least 28% and specificity is at least 94%.
248. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 3500 and identified at nucleotide positions 61 to 62, and wherein the sensitivity is at least 26% and specificity is at least 95%.
249. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 4000 and identified at nucleotide positions 61 to 62, and wherein the sensitivity is at least 29% and specificity is at least 95%.
250. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 4500 and identified at nucleotide positions 61 to 62, and wherein the sensitivity is at least 32% and specificity is at least 94%.
251. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 5000 and identified at nucleotide positions 61 to 62, and wherein the sensitivity is at least 29% and specificity is at least 96%.
252. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 5500 and identified at nucleotide positions 61 to 62, and wherein the sensitivity is at least 27% and specificity is at least 95%.
253. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 6000 and identified at nucleotide positions 61 to 62, and wherein the sensitivity is at least 27% and specificity is at least 96%.
254. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 6500 and identified at nucleotide positions 61 to 62, and wherein the sensitivity is at least 31% and specificity is at least 96%.
255. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 7000 and identified at nucleotide positions 61 to 62, and wherein the sensitivity is at least 29% and specificity is at least 96%.
256. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 7500 and identified at nucleotide positions 61 to 62, and wherein the sensitivity is at least 29% and specificity is at least 96%.
257. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 8000 and identified at nucleotide positions 61 to 62, and wherein the sensitivity is at least 29% and specificity is at least 96%.
258. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 8500 and identified at nucleotide positions 61 to 62, and wherein the sensitivity is at least 29% and specificity is at least 96%.
259. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 9000 and identified at nucleotide positions 61 to 62, and wherein the sensitivity is at least 29% and specificity is at least 96%.
260. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 9500 and identified at nucleotide positions 61 to 62, and wherein the sensitivity is at least 28% and specificity is at least 95%.
261. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 10000 and identified at nucleotide positions 61 to 62, and wherein the sensitivity is at least 29% and specificity is at least 95%.
262. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 11000 and identified at nucleotide positions 61 to 62, and wherein the sensitivity is at least 29% and specificity is at least 96%.
263. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 12000 and identified at nucleotide positions 61 to 62, and wherein the sensitivity is at least 28% and specificity is at least 96%.
264. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 13000 and identified at nucleotide positions 61 to 62, and wherein the sensitivity is at least 28% and specificity is at least 96%.
265. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 14000 and identified at nucleotide positions 61 to 62, and wherein the sensitivity is at least 27% and specificity is at least 96%.
266. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 15000 and identified at nucleotide positions 61 to 62, and wherein the sensitivity is at least 28% and specificity is at least 96%.
267. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 16000 and identified at nucleotide positions 61 to 62, and wherein the sensitivity is at least 27% and specificity is at least 96%.
268. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 17000 and identified at nucleotide positions 61 to 62, and wherein the sensitivity is at least 32% and specificity is at least 96%.
269. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 18000 and identified at nucleotide positions 61 to 62, and wherein the sensitivity is at least 33% and specificity is at least 96%.
270. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 19000 and identified at nucleotide positions 61 to 62, and wherein the sensitivity is at least 34% and specificity is at least 96%.
271. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 20000 and identified at nucleotide positions 61 to 62, and wherein the sensitivity is at least 34% and specificity is at least 96%.
272. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 22000 and identified at nucleotide positions 61 to 62, and wherein the sensitivity is at least 34% and specificity is at least 97%.
273. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 24000 and identified at nucleotide positions 61 to 62, and wherein the sensitivity is at least 33% and specificity is at least 97%.
274. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 26000 and identified at nucleotide positions 61 to 62, and wherein the sensitivity is at least 34% and specificity is at least 97%.
275. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 28000 and identified at nucleotide positions 61 to 62, and wherein the sensitivity is at least 34% and specificity is at least 97%.
276. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 30000 and identified at nucleotide positions 61 to 62, and wherein the sensitivity is at least 33% and specificity is at least 97%.
277. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 32000 and identified at nucleotide positions 61 to 62, and wherein the sensitivity is at least 32% and specificity is at least 97%.
278. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 34000 and identified at nucleotide positions 61 to 62, and wherein the sensitivity is at least 33% and specificity is at least 97%.
279. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 36000 and identified at nucleotide positions 61 to 62, and wherein the sensitivity is at least 33% and specificity is at least 97%.
280. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 38000 and identified at nucleotide positions 61 to 62, and wherein the sensitivity is at least 33% and specificity is at least 97%.
281. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 40753 and identified at nucleotide positions 61 to 62, and wherein the sensitivity is at least 34% and specificity is at least 97%.
282. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 500 to 40753 and identified at nucleotide positions 61 to 62, and wherein the sensitivity is at least 94% and specificity is at least 51%.
283. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1000 to 40753 and identified at nucleotide positions 61 to 62, and wherein the sensitivity is at least 92% and specificity is at least 51%.
284. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1500 to 40753 and identified at nucleotide positions 61 to 62, and wherein the sensitivity is at least 91% and specificity is at least 53%.
285. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 2000 to 40753 and identified at nucleotide positions 61 to 62, and wherein the sensitivity is at least 91% and specificity is at least 53%.
286. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 2500 to 40753 and identified at nucleotide positions 61 to 62, and wherein the sensitivity is at least 91% and specificity is at least 54%.
287. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 3000 to 40753 and identified at nucleotide positions 61 to 62, and wherein the sensitivity is at least 91% and specificity is at least 54%.
288. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 3500 to 40753 and identified at nucleotide positions 61 to 62, and wherein the sensitivity is at least 87% and specificity is at least 56%.
289. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 4000 to 40753 and identified at nucleotide positions 61 to 62, and wherein the sensitivity is at least 86% and specificity is at least 56%.
290. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 4500 to 40753 and identified at nucleotide positions 61 to 62, and wherein the sensitivity is at least 86% and specificity is at least 57%.
291. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 5000 to 40753 and identified at nucleotide positions 61 to 62, and wherein the sensitivity is at least 86% and specificity is at least 56%.
292. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 5500 to 40753 and identified at nucleotide positions 61 to 62, and wherein the sensitivity is at least 80% and specificity is at least 57%.
293. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 6000 to 40753 and identified at nucleotide positions 61 to 62, and wherein the sensitivity is at least 78% and specificity is at least 57%.
294. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 6500 to 40753 and identified at nucleotide positions 61 to 62, and wherein the sensitivity is at least 78% and specificity is at least 57%.
295. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 7000 to 40753 and identified at nucleotide positions 61 to 62, and wherein the sensitivity is at least 78% and specificity is at least 57%.
296. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 7500 to 40753 and identified at nucleotide positions 61 to 62, and wherein the sensitivity is at least 77% and specificity is at least 57%.
297. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 8000 to 40753 and identified at nucleotide positions 61 to 62, and wherein the sensitivity is at least 77% and specificity is at least 56%.
298. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 8500 to 40753 and identified at nucleotide positions 61 to 62, and wherein the sensitivity is at least 78% and specificity is at least 56%.
299. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 9000 to 40753 and identified at nucleotide positions 61 to 62, and wherein the sensitivity is at least 75% and specificity is at least 56%.
300. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 9500 to 40753 and identified at nucleotide positions 61 to 62, and wherein the sensitivity is at least 75% and specificity is at least 57%.
301. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 10000 to 40753 and identified at nucleotide positions 61 to 62, and wherein the sensitivity is at least 76% and specificity is at least 57%.
302. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 11000 to 40753 and identified at nucleotide positions 61 to 62, and wherein the sensitivity is at least 76% and specificity is at least 57%.
303. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 12000 to 40753 and identified at nucleotide positions 61 to 62, and wherein the sensitivity is at least 76% and specificity is at least 59%.
304. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 13000 to 40753 and identified at nucleotide positions 61 to 62, and wherein the sensitivity is at least 75% and specificity is at least 59%.
305. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 14000 to 40753 and identified at nucleotide positions 61 to 62, and wherein the sensitivity is at least 68% and specificity is at least 59%.
306. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 15000 to 40753 and identified at nucleotide positions 61 to 62, and wherein the sensitivity is at least 69% and specificity is at least 58%.
307. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 16000 to 40753 and identified at nucleotide positions 61 to 62, and wherein the sensitivity is at least 65% and specificity is at least 60%.
308. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 17000 to 40753 and identified at nucleotide positions 61 to 62, and wherein the sensitivity is at least 65% and specificity is at least 60%.
309. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 18000 to 40753 and identified at nucleotide positions 61 to 62, and wherein the sensitivity is at least 64% and specificity is at least 61%.
310. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 19000 to 40753 and identified at nucleotide positions 61 to 62, and wherein the sensitivity is at least 63% and specificity is at least 61%.
311. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 20000 to 40753 and identified at nucleotide positions 61 to 62, and wherein the sensitivity is at least 63% and specificity is at least 6%.
312. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 22000 to 40753 and identified at nucleotide positions 61 to 62, and wherein the sensitivity is at least 65% and specificity is at least 61%.
313. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 24000 to 40753 and identified at nucleotide positions 61 to 62, and wherein the sensitivity is at least 67% and specificity is at least 61%.
314. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 26000 to 40753 and identified at nucleotide positions 61 to 62, and wherein the sensitivity is at least 67% and specificity is at least 58%.
315. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 28000 to 40753 and identified at nucleotide positions 61 to 62, and wherein the sensitivity is at least 63% and specificity is at least 60%.
316. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 30000 to 40753 and identified at nucleotide positions 61 to 62, and wherein the sensitivity is at least 63% and specificity is at least 60%.
317. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 32000 to 40753 and identified at nucleotide positions 61 to 62, and wherein the sensitivity is at least 61% and specificity is at least 59%.
318. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 34000 to 40753 and identified at nucleotide positions 61 to 62, and wherein the sensitivity is at least 63% and specificity is at least 59%.
319. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 36000 to 40753 and identified at nucleotide positions 61 to 62, and wherein the sensitivity is at least 60% and specificity is at least 60%.
320. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 38000 to 40753 and identified at nucleotide positions 61 to 62, and wherein the sensitivity is at least 61% and specificity is at least 58%.
321. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 500 to 40753 and identified at nucleotide positions 61 to 62, and wherein the sensitivity is at least 77% and specificity is at least 79%.
322. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1000 to 40753 and identified at nucleotide positions 61 to 62, and wherein the sensitivity is at least 79% and specificity is at least 78%.
323. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1500 to 40753 and identified at nucleotide positions 61 to 62, and wherein the sensitivity is at least 73% and specificity is at least 74%.
324. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 2000 to 40753 and identified at nucleotide positions 61 to 62, and wherein the sensitivity is at least 75% and specificity is at least 74%.
325. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 2500 to 40753 and identified at nucleotide positions 61 to 62, and wherein the sensitivity is at least 72% and specificity is at least 74%.
326. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 3000 to 40753 and identified at nucleotide positions 61 to 62, and wherein the sensitivity is at least 72% and specificity is at least 74%.
327. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 3500 to 40753 and identified at nucleotide positions 61 to 62, and wherein the sensitivity is at least 73% and specificity is at least 74%.
328. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 4000 to 40753 and identified at nucleotide positions 61 to 62, and wherein the sensitivity is at least 69% and specificity is at least 74%.
329. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 4500 to 40753 and identified at nucleotide positions 61 to 62, and wherein the sensitivity is at least 71% and specificity is at least 74%.
330. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 5000 to 40753 and identified at nucleotide positions 61 to 62, and wherein the sensitivity is at least 69% and specificity is at least 74%.
331. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 5500 to 40753 and identified at nucleotide positions 61 to 62, and wherein the sensitivity is at least 66% and specificity is at least 74%.
332. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 6000 to 40753 and identified at nucleotide positions 61 to 62, and wherein the sensitivity is at least 61% and specificity is at least 75%.
333. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 6500 to 40753 and identified at nucleotide positions 61 to 62, and wherein the sensitivity is at least 61% and specificity is at least 75%.
334. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 7000 to 40753 and identified at nucleotide positions 61 to 62, and wherein the sensitivity is at least 60% and specificity is at least 75%.
335. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 7500 to 40753 and identified at nucleotide positions 61 to 62, and wherein the sensitivity is at least 61% and specificity is at least 74%.
336. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 8000 to 40753 and identified at nucleotide positions 61 to 62, and wherein the sensitivity is at least 59% and specificity is at least 74%.
337. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 8500 to 40753 and identified at nucleotide positions 61 to 62, and wherein the sensitivity is at least 60% and specificity is at least 74%.
338. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 9000 to 40753 and identified at nucleotide positions 61 to 62, and wherein the sensitivity is at least 58% and specificity is at least 75%.
339. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 9500 to 40753 and identified at nucleotide positions 61 to 62, and wherein the sensitivity is at least 58% and specificity is at least 75%.
340. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 10000 to 40753 and identified at nucleotide positions 61 to 62, and wherein the sensitivity is at least 58% and specificity is at least 74%.
341. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 11000 to 40753 and identified at nucleotide positions 61 to 62, and wherein the sensitivity is at least 58% and specificity is at least 75%.
342. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 12000 to 40753 and identified at nucleotide positions 61 to 62, and wherein the sensitivity is at least 59% and specificity is at least 75%.
343. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 13000 to 40753 and identified at nucleotide positions 61 to 62, and wherein the sensitivity is at least 59% and specificity is at least 74%.
344. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 14000 to 40753 and identified at nucleotide positions 61 to 62, and wherein the sensitivity is at least 53% and specificity is at least 75%.
345. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 15000 to 40753 and identified at nucleotide positions 61 to 62, and wherein the sensitivity is at least 54% and specificity is at least 74%.
346. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 16000 to 40753 and identified at nucleotide positions 61 to 62, and wherein the sensitivity is at least 53% and specificity is at least 75%.
347. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 17000 to 40753 and identified at nucleotide positions 61 to 62, and wherein the sensitivity is at least 47% and specificity is at least 75%.
348. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 18000 to 40753 and identified at nucleotide positions 61 to 62, and wherein the sensitivity is at least 47% and specificity is at least 75%.
349. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 19000 to 40753 and identified at nucleotide positions 61 to 62, and wherein the sensitivity is at least 43% and specificity is at least 75%.
350. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 20000 to 40753 and identified at nucleotide positions 61 to 62, and wherein the sensitivity is at least 43% and specificity is at least 76%.
351. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 22000 to 40753 and identified at nucleotide positions 61 to 62, and wherein the sensitivity is at least 49% and specificity is at least 74%.
352. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 24000 to 40753 and identified at nucleotide positions 61 to 62, and wherein the sensitivity is at least 51% and specificity is at least 75%.
353. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 26000 to 40753 and identified at nucleotide positions 61 to 62, and wherein the sensitivity is at least 51% and specificity is at least 75%.
354. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 28000 to 40753 and identified at nucleotide positions 61 to 62, and wherein the sensitivity is at least 43% and specificity is at least 76%.
355. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 30000 to 40753 and identified at nucleotide positions 61 to 62, and wherein the sensitivity is at least 39% and specificity is at least 76%.
356. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 32000 to 40753 and identified at nucleotide positions 61 to 62, and wherein the sensitivity is at least 38% and specificity is at least 76%.
357. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 34000 to 40753 and identified at nucleotide positions 61 to 62, and wherein the sensitivity is at least 37% and specificity is at least 76%.
358. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 36000 to 40753 and identified at nucleotide positions 61 to 62, and wherein the sensitivity is at least 37% and specificity is at least 77%.
359. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 38000 to 40753 and identified at nucleotide positions 61 to 62, and wherein the sensitivity is at least 35% and specificity is at least 76%.
360. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 500 to 40753 and identified at nucleotide positions 61 to 62, and wherein the sensitivity is at least 35% and specificity is at least 97%.
361. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1000 to 40753 and identified at nucleotide positions 61 to 62, and wherein the sensitivity is at least 34% and specificity is at least 96%.
362. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1500 to 40753 and identified at nucleotide positions 61 to 62, and wherein the sensitivity is at least 38% and specificity is at least 94%.
363. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 2000 to 40753 and identified at nucleotide positions 61 to 62, and wherein the sensitivity is at least 39% and specificity is at least 94%.
364. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 2500 to 40753 and identified at nucleotide positions 61 to 62, and wherein the sensitivity is at least 41% and specificity is at least 92%.
365. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 3000 to 40753 and identified at nucleotide positions 61 to 62, and wherein the sensitivity is at least 40% and specificity is at least 90%.
366. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 3500 to 40753 and identified at nucleotide positions 61 to 62, and wherein the sensitivity is at least 42% and specificity is at least 90%.
367. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 4000 to 40753 and identified at nucleotide positions 61 to 62, and wherein the sensitivity is at least 41% and specificity is at least 90%.
368. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 4500 to 40753 and identified at nucleotide positions 61 to 62, and wherein the sensitivity is at least 42% and specificity is at least 90%.
369. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 5000 to 40753 and identified at nucleotide positions 61 to 62, and wherein the sensitivity is at least 42% and specificity is at least 89%.
370. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 5500 to 40753 and identified at nucleotide positions 61 to 62, and wherein the sensitivity is at least 36% and specificity is at least 89%.
371. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 6000 to 40753 and identified at nucleotide positions 61 to 62, and wherein the sensitivity is at least 33% and specificity is at least 89%.
372. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 6500 to 40753 and identified at nucleotide positions 61 to 62, and wherein the sensitivity is at least 32% and specificity is at least 89%.
373. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 7000 to 40753 and identified at nucleotide positions 61 to 62, and wherein the sensitivity is at least 32% and specificity is at least 89%.
374. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 7500 to 40753 and identified at nucleotide positions 61 to 62, and wherein the sensitivity is at least 33% and specificity is at least 88%.
375. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 8000 to 40753 and identified at nucleotide positions 61 to 62, and wherein the sensitivity is at least 32% and specificity is at least 88%.
376. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 8500 to 40753 and identified at nucleotide positions 61 to 62, and wherein the sensitivity is at least 32% and specificity is at least 86%.
377. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 9000 to 40753 and identified at nucleotide positions 61 to 62, and wherein the sensitivity is at least 31% and specificity is at least 87%.
378. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 9500 to 40753 and identified at nucleotide positions 61 to 62, and wherein the sensitivity is at least 31% and specificity is at least 87%.
379. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 10000 to 40753 and identified at nucleotide positions 61 to 62, and wherein the sensitivity is at least 29% and specificity is at least 86%.
380. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 11000 to 40753 and identified at nucleotide positions 61 to 62, and wherein the sensitivity is at least 29% and specificity is at least 86%.
381. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 12000 to 40753 and identified at nucleotide positions 61 to 62, and wherein the sensitivity is at least 28% and specificity is at least 86%.
382. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 13000 to 40753 and identified at nucleotide positions 61 to 62, and wherein the sensitivity is at least 29% and specificity is at least 86%.
383. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 14000 to 40753 and identified at nucleotide positions 61 to 62, and wherein the sensitivity is at least 27% and specificity is at least 87%.
384. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 15000 to 40753 and identified at nucleotide positions 61 to 62, and wherein the sensitivity is at least 28% and specificity is at least 87%.
385. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 16000 to 40753 and identified at nucleotide positions 61 to 62, and wherein the sensitivity is at least 26% and specificity is at least 87%.
386. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 17000 to 40753 and identified at nucleotide positions 61 to 62, and wherein the sensitivity is at least 26% and specificity is at least 87%.
387. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 18000 to 40753 and identified at nucleotide positions 61 to 62, and wherein the sensitivity is at least 26% and specificity is at least 87%.
388. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 19000 to 40753 . . . and identified at nucleotide positions 61 to 62, and wherein the sensitivity is at least 25% and specificity is at least 88%.
389. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 20000 to 40753 and identified at nucleotide positions 61 to 62, and wherein the sensitivity is at least 25% and specificity is at least 88%.
390. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 22000 to 40753 and identified at nucleotide positions 61 to 62, and wherein the sensitivity is at least 25% and specificity is at least 87%.
391. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 24000 to 40753 and identified at nucleotide positions 61 to 62, and wherein the sensitivity is at least 25% and specificity is at least 86%.
392. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 26000 to 40753 and identified at nucleotide positions 61 to 62, and wherein the sensitivity is at least 25% and specificity is at least 86%.
393. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 28000 to 40753 and identified at nucleotide positions 61 to 62, and wherein the sensitivity is at least 24% and specificity is at least 88%.
394. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 30000 to 40753 and identified at nucleotide positions 61 to 62, and wherein the sensitivity is at least 25% and specificity is at least 88%.
395. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 32000 to 40753 and identified at nucleotide positions 61 to 62, and wherein the sensitivity is at least 24% and specificity is at least 87%.
396. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 34000 to 40753 and identified at nucleotide positions 61 to 62, and wherein the sensitivity is at least 24% and specificity is at least 87%.
397. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 36000 to 40753 and identified at nucleotide positions 61 to 62, and wherein the sensitivity is at least 24% and specificity is at least 87%.
398. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 38000 to 40752 and identified at nucleotide positions 61 to 62, and wherein the sensitivity is at least 24% and specificity is at least 87%.
399. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1 to 500 and identified at nucleotide positions 61 to 62, and wherein the sensitivity is at least 85% and specificity is at least 52%.
400. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 501 to 1000 and identified at nucleotide positions 61 to 62, and wherein the sensitivity is at least 86% and specificity is at least 49%.
401. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1001 to 1500 and identified at nucleotide positions 61 to 62, and wherein the sensitivity is at least 81% and specificity is at least 50%.
402. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 1501 to 2000 and identified at nucleotide positions 61 to 62, and wherein the sensitivity is at least 79% and specificity is at least 51%.
403. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 2001 to 2500 and identified at nucleotide positions 61 to 62, and wherein the sensitivity is at least 81% and specificity is at least 48%.
404. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 2501 to 3000 and identified at nucleotide positions 61 to 62, and wherein the sensitivity is at least 78% and specificity is at least 47%.
405. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 3001 to 3500 and identified at nucleotide positions 61 to 62, and wherein the sensitivity is at least 79% and specificity is at least 48%.
406. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 3501 to 4000 and identified at nucleotide positions 61 to 62, and wherein the sensitivity is at least 72% and specificity is at least 46%.
407. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 4001 to 4500 and identified at nucleotide positions 61 to 62, and wherein the sensitivity is at least 81% and specificity is at least 47%.
408. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 4501 to 5000 and identified at nucleotide positions 61 to 62, and wherein the sensitivity is at least 76% and specificity is at least 44%.
409. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 5001 to 5500 and identified at nucleotide positions 61 to 62, and wherein the sensitivity is at least 82% and specificity is at least 45%.
410. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 5501 to 6000 and identified at nucleotide positions 61 to 62, and wherein the sensitivity is at least 73% and specificity is at least 53%.
411. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 6001 to 6500 and identified at nucleotide positions 61 to 62, and wherein the sensitivity is at least 84% and specificity is at least 50%.
412. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 6501 to 7000 and identified at nucleotide positions 61 to 62, and wherein the sensitivity is at least 86% and specificity is at least 49%.
413. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 7001 to 7500 and identified at nucleotide positions 61 to 62, and wherein the sensitivity is at least 75% and specificity is at least 50%.
414. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 7501 to 8000 and identified at nucleotide positions 61 to 62, and wherein the sensitivity is at least 85% and specificity is at least 49%.
415. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 8001 to 8500 and identified at nucleotide positions 61 to 62, and wherein the sensitivity is at least 78% and specificity is at least 51%.
416. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 8501 to 9000 and identified at nucleotide positions 61 to 62, and wherein the sensitivity is at least 82% and specificity is at least 53%.
417. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 9001 to 9500 and identified at nucleotide positions 61 to 62, and wherein the sensitivity is at least 78% and specificity is at least 54%.
418. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 9501 to 10000 and identified at nucleotide positions 61 to 62, and wherein the sensitivity is at least 75% and specificity is at least 55%.
419. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 10001 to 10500 and identified at nucleotide positions 61 to 62, and wherein the sensitivity is at least 80% and specificity is at least 48%.
420. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 10501 to 11000 and identified at nucleotide positions 61 to 62, and wherein the sensitivity is at least 75% and specificity is at least 56%.
421. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 11001 to 11500 and identified at nucleotide positions 61 to 62, and wherein the sensitivity is at least 86% and specificity is at least 51%.
422. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 11501 to 12000 and identified at nucleotide positions 61 to 62, and wherein the sensitivity is at least 76% and specificity is at least 45%.
423. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 12001 to 12500 and identified at nucleotide positions 61 to 62, and wherein the sensitivity is at least 74% and specificity is at least 54%.
424. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 12501 to 13000 and identified at nucleotide positions 61 to 62, and wherein the sensitivity is at least 80% and specificity is at least 53%.
425. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 13001 to 13500 and identified at nucleotide positions 61 to 62, and wherein the sensitivity is at least 77% and specificity is at least 56%.
426. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 13001 to 13500 and identified at nucleotide positions 61 to 62, and wherein the sensitivity is at least 77% and specificity is at least 56%.
427. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 13501 to 14000 and identified at nucleotide positions 61 to 62, and wherein the sensitivity is at least 75% and specificity is at least 55%.
428. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 14001 to 14500 and identified at nucleotide positions 61 to 62, and wherein the sensitivity is at least 74% and specificity is at least 55%.
429. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 14501 to 15000 and identified at nucleotide positions 61 to 62, and wherein the sensitivity is at least 80% and specificity is at least 52%.
430. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 15001 to 15500 and identified at nucleotide positions 61 to 62, and wherein the sensitivity is at least 75% and specificity is at least 52%.
431. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 15501 to 16000 and identified at nucleotide positions 61 to 62, and wherein the sensitivity is at least 75% and specificity is at least 57%.
432. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 16001 to 16500 and identified at nucleotide positions 61 to 62, and wherein the sensitivity is at least 79% and specificity is at least 53%.
433. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 16501 to 17000 and identified at nucleotide positions 61 to 62, and wherein the sensitivity is at least 74% and specificity is at least 56%.
434. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 17001 to 17500 and identified at nucleotide positions 61 to 62, and wherein the sensitivity is at least 77% and specificity is at least 53%.
435. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 17501 to 18000 and identified at nucleotide positions 61 to 62, and wherein the sensitivity is at least 72% and specificity is at least 54%.
436. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 18001 to 18500 and identified at nucleotide positions 61 to 62, and wherein the sensitivity is at least 77% and specificity is at least 56%.
437. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 18501 to 19000 and identified at nucleotide positions 61 to 62, and wherein the sensitivity is at least 76% and specificity is at least 57%.
438. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 19001 to 19500 and identified at nucleotide positions 61 to 62, and wherein the sensitivity is at least 73% and specificity is at least 57%.
439. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 19501 to 20000 and identified at nucleotide positions 61 to 62, and wherein the sensitivity is at least 74% and specificity is at least 54%.
440. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 20001 to 20500 and identified at nucleotide positions 61 to 62, and wherein the sensitivity is at least 72% and specificity is at least 56%.
441. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 20501 to 21000 and identified at nucleotide positions 61 to 62, and wherein the sensitivity is at least 77% and specificity is at least 59%.
442. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 21001 to 21500 and identified at nucleotide positions 61 to 62, and wherein the sensitivity is at least 66% and specificity is at least 54%.
443. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 21501 to 22000 and identified at nucleotide positions 61 to 62, and wherein the sensitivity is at least 72% and specificity is at least 57%.
444. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 22001 to 22500 and identified at nucleotide positions 61 to 62, and wherein the sensitivity is at least 71% and specificity is at least 56%.
445. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 22501 to 23000 and identified at nucleotide positions 61 to 62, and wherein the sensitivity is at least 72% and specificity is at least 57%.
446. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 23001 to 23500 and identified at nucleotide positions 61 to 62, and wherein the sensitivity is at least 64% and specificity is at least 57%.
447. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 23501 to 24000 and identified at nucleotide positions 61 to 62, and wherein the sensitivity is at least 68% and specificity is at least 61%.
448. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 24001 to 24500 and identified at nucleotide positions 61 to 62, and wherein the sensitivity is at least 69% and specificity is at least 57%.
449. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 24501 to 25000 and identified at nucleotide positions 61 to 62, and wherein the sensitivity is at least 65% and specificity is at least 59%.
450. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 25001 to 25500 and identified at nucleotide positions 61 to 62, and wherein the sensitivity is at least 62% and specificity is at least 58%.
451. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 25501 to 26000 and identified at nucleotide positions 61 to 62, and wherein the sensitivity is at least 63% and specificity is at least 56%.
452. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 26001 to 26500 and identified at nucleotide positions 61 to 62, and wherein the sensitivity is at least 69% and specificity is at least 59%.
453. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 26501 to 27000 and identified at nucleotide positions 61 to 62, and wherein the sensitivity is at least 66% and specificity is at least 58%.
454. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 27001 to 27500 and identified at nucleotide positions 61 to 62, and wherein the sensitivity is at least 67% and specificity is at least 59%.
455. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 27501 to 28000 and identified at nucleotide positions 61 to 62, and wherein the sensitivity is at least 62% and specificity is at least 60%.
456. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 28001 to 28500 and identified at nucleotide positions 61 to 62, and wherein the sensitivity is at least 61% and specificity is at least 59%.
457. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 28501 to 29000 and identified at nucleotide positions 61 to 62, and wherein the sensitivity is at least 65% and specificity is at least 61%.
458. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 29001 to 29500 and identified at nucleotide positions 61 to 62, and wherein the sensitivity is at least 65% and specificity is at least 58%.
459. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 29501 to 30000 and identified at nucleotide positions 61 to 62, and wherein the sensitivity is at least 67% and specificity is at least 55%.
460. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 30001 to 30500 and identified at nucleotide positions 61 to 62, and wherein the sensitivity is at least 65% and specificity is at least %.
461. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 30501 to 31000 and identified at nucleotide positions 61 to 62, and wherein the sensitivity is at least 63% and specificity is at least 61%.
462. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 31001 to 31500 and identified at nucleotide positions 61 to 62, and wherein the sensitivity is at least 62% and specificity is at least 59%.
463. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 31501 to 32000 and identified at nucleotide positions 61 to 62, and wherein the sensitivity is at least 68% and specificity is at least 56%.
464. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 32001 to 32500 and identified at nucleotide positions 61 to 62, and wherein the sensitivity is at least 62% and specificity is at least 59%.
465. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 32501 to 33000 and identified at nucleotide positions 61 to 62, and wherein the sensitivity is at least 67% and specificity is at least 58%.
466. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 33001 to 33500 and identified at nucleotide positions 61 to 62, and wherein the sensitivity is at least 65% and specificity is at least 56%.
467. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 33501 to 34000 and identified at nucleotide positions 61 to 62, and wherein the sensitivity is at least 61% and specificity is at least 59%.
468. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 34001 to 34500 and identified at nucleotide positions 61 to 62, and wherein the sensitivity is at least 58% and specificity is at least 60%.
469. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 34501 to 35000 and identified at nucleotide positions 61 to 62, and wherein the sensitivity is at least 59% and specificity is at least 57%.
470. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 35001 to 35500 and identified at nucleotide positions 61 to 62, and wherein the sensitivity is at least 60% and specificity is at least 61%.
471. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 35501 to 36000 and identified at nucleotide positions 61 to 62, and wherein the sensitivity is at least 61% and specificity is at least 57%.
472. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 36001 to 36500 and identified at nucleotide positions 61 to 62, and wherein the sensitivity is at least 61% and specificity is at least 57%.
473. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 36501 to 37000 and identified at nucleotide positions 61 to 62, and wherein the sensitivity is at least 63% and specificity is at least 62%.
474. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 37001 to 37500 and identified at nucleotide positions 61 to 62, and wherein the sensitivity is at least 62% and specificity is at least 60%.
475. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 37501 to 38000 and identified at nucleotide positions 61 to 62, and wherein the sensitivity is at least 60% and specificity is at least 61%.
476. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 38001 to 38500 and identified at nucleotide positions 61 to 62, and wherein the sensitivity is at least 61% and specificity is at least 57%.
477. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 38501 to 39000 and identified at nucleotide positions 61 to 62, and wherein the sensitivity is at least 61% and specificity is at least 57%.
478. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 39001 to 39500 and identified at nucleotide positions 61 to 62, and wherein the sensitivity is at least 63% and specificity is at least 59%.
479. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 39501 to 40000 and identified at nucleotide positions 61 to 62, and wherein the sensitivity is at least 60% and specificity is at least 59%.
480. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 40001 to 40500 and identified at nucleotide positions 61 to 62, and wherein the sensitivity is at least 62% and specificity is at least 59%.
481. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 40501 to 40753 and identified at nucleotide positions 61 to 62, and wherein the sensitivity is at least 61% and specificity is at least 60%.
482. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1 to 500 and identified at nucleotide positions 61 to 62, and wherein the sensitivity is at least 63% and specificity is at least 69%.
483. An assay according to the invention, wherein the WID-BC-Index for the individual is about −0.235 or more, the individual is classified as having at least a low risk of harbouring breast cancer or a low risk of breast cancer development, wherein the set of CpGs comprises at least the CpGs defined by SEQ ID NOs 501 to 1000 and identified at nucleotide positions 61 to 62, and wherein the sensitivity is at least 66% and specificity is at least 67%.
484. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1001 to 1500 and identified at nucleotide positions 61 to 62, and wherein the sensitivity is at least 67% and specificity is at least 74%.
485. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 1501 to 2000 and identified at nucleotide positions 61 to 62, and wherein the sensitivity is at least 58% and specificity is at least 72%.
486. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 2001 to 2500 and identified at nucleotide positions 61 to 62, and wherein the sensitivity is at least 68% and specificity is at least 68%.
487. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 2501 to 3000 and identified at nucleotide positions 61 to 62, and wherein the sensitivity is at least 63% and specificity is at least 67%.
488. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 3001 to 3500 and identified at nucleotide positions 61 to 62, and wherein the sensitivity is at least 65% and specificity is at least 66%.
489. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 3501 to 4000 and identified at nucleotide positions 61 to 62, and wherein the sensitivity is at least 64% and specificity is at least 67%.
490. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 4001 to 4500 and identified at nucleotide positions 61 to 62, and wherein the sensitivity is at least 71% and specificity is at least 64%.
491. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 4501 to 5000 and identified at nucleotide positions 61 to 62, and wherein the sensitivity is at least 62% and specificity is at least 66%.
492. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 5001 to 5500 and identified at nucleotide positions 61 to 62, and wherein the sensitivity is at least 66% and specificity is at least 63%.
493. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 5501 to 6000 and identified at nucleotide positions 61 to 62, and wherein the sensitivity is at least 57% and specificity is at least 74%.
494. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 6001 to 6500 and identified at nucleotide positions 61 to 62, and wherein the sensitivity is at least 71% and specificity is at least 71%.
495. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 6501 to 7000 and identified at nucleotide positions 61 to 62, and wherein the sensitivity is at least 72% and specificity is at least 68%.
496. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 7001 to 7500 and identified at nucleotide positions 61 to 62, and wherein the sensitivity is at least 54% and specificity is at least 73%.
497. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 7501 to 8000 and identified at nucleotide positions 61 to 62, and wherein the sensitivity is at least 76% and specificity is at least 69%.
498. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 8001 to 8500 and identified at nucleotide positions 61 to 62, and wherein the sensitivity is at least 62% and specificity is at least 67%.
499. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 8501 to 9000 and identified at nucleotide positions 61 to 62, and wherein the sensitivity is at least 62% and specificity is at least 69%.
500. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 9001 to 9500 and identified at nucleotide positions 61 to 62, and wherein the sensitivity is at least 64% and specificity is at least 69%.
501. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 9501 to 10000 and identified at nucleotide positions 61 to 62, and wherein the sensitivity is at least 62% and specificity is at least 70%.
502. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 10001 to 10500 and identified at nucleotide positions 61 to 62, and wherein the sensitivity is at least 63% and specificity is at least 69%.
503. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 10501 to 11000 and identified at nucleotide positions 61 to 62, and wherein the sensitivity is at least 59% and specificity is at least 72%.
504. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 110001 to 11500 and identified at nucleotide positions 61 to 62, and wherein the sensitivity is at least 73% and specificity is at least 64%.
505. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 11501 to 12000 and identified at nucleotide positions 61 to 62, and wherein the sensitivity is at least 65% and specificity is at least 64%.
506. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 12001 to 12500 and identified at nucleotide positions 61 to 62, and wherein the sensitivity is at least 60% and specificity is at least 70%.
507. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 12501 to 13000 and identified at nucleotide positions 61 to 62, and wherein the sensitivity is at least 66% and specificity is at least 68%.
508. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 13001 to 13500 and identified at nucleotide positions 61 to 62, and wherein the sensitivity is at least 67% and specificity is at least 69%.
509. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 13501 to 14000 and identified at nucleotide positions 61 to 62, and wherein the sensitivity is at least 64% and specificity is at least 69%.
510. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 14001 to 14500 and identified at nucleotide positions 61 to 62, and wherein the sensitivity is at least 62% and specificity is at least 69%.
511. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 14501 to 15000 and identified at nucleotide positions 61 to 62, and wherein the sensitivity is at least 67% and specificity is at least 68%.
512. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 15001 to 15500 and identified at nucleotide positions 61 to 62, and wherein the sensitivity is at least 62% and specificity is at least 67%.
513. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 15501 to 16000 and identified at nucleotide positions 61 to 62, and wherein the sensitivity is at least 64% and specificity is at least 71%.
514. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 16001 to 16500 and identified at nucleotide positions 61 to 62, and wherein the sensitivity is at least 64% and specificity is at least 68%.
515. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 16501 to 17000 and identified at nucleotide positions 61 to 62, and wherein the sensitivity is at least 62% and specificity is at least 70%.
516. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 17001 to 17500 and identified at nucleotide positions 61 to 62, and wherein the sensitivity is at least 64% and specificity is at least 67%.
517. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 17501 to 18000 and identified at nucleotide positions 61 to 62, and wherein the sensitivity is at least 57% and specificity is at least 71%.
518. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 18001 to 18500 and identified at nucleotide positions 61 to 62, and wherein the sensitivity is at least 66% and specificity is at least 71%.
519. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 18501 to 19000 and identified at nucleotide positions 61 to 62, and wherein the sensitivity is at least 49% and specificity is at least 71%.
520. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 19001 to 19500 and identified at nucleotide positions 61 to 62, and wherein the sensitivity is at least 49% and specificity is at least 73%.
521. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 19501 to 20000 and identified at nucleotide positions 61 to 62, and wherein the sensitivity is at least 54% and specificity is at least 73%.
522. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 20001 to 20500 and identified at nucleotide positions 61 to 62, and wherein the sensitivity is at least 56% and specificity is at least 72%.
523. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 20501 to 21000 and identified at nucleotide positions 61 to 62, and wherein the sensitivity is at least 59% and specificity is at least 73%.
524. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 21001 to 21500 and identified at nucleotide positions 61 to 62, and wherein the sensitivity is at least 54% and specificity is at least 72%.
525. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 21501 to 22000 and identified at nucleotide positions 61 to 62, and wherein the sensitivity is at least 56% and specificity is at least 74%.
526. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 22001 to 22500 and identified at nucleotide positions 61 to 62, and wherein the sensitivity is at least 51% and specificity is at least 74%.
527. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 22501 to 23000 and identified at nucleotide positions 61 to 62, and wherein the sensitivity is at least 54% and specificity is at least 74%.
528. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 23001 to 23500 and identified at nucleotide positions 61 to 62, and wherein the sensitivity is at least 53% and specificity is at least 75%.
529. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 23501 to 24000 and identified at nucleotide positions 61 to 62, and wherein the sensitivity is at least 51% and specificity is at least 72%.
530. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 24001 to 24500 and identified at nucleotide positions 61 to 62, and wherein the sensitivity is at least 49% and specificity is at least 72%.
531. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 24501 to 25000 and identified at nucleotide positions 61 to 62, and wherein the sensitivity is at least 49% and specificity is at least 77%.
532. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 24501 to 25000 and identified at nucleotide positions 61 to 62, and wherein the sensitivity is at least 49% and specificity is at least 77%.
533. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 25001 to 25500 and identified at nucleotide positions 61 to 62, and wherein the sensitivity is at least 44% and specificity is at least 75%.
534. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 25501 to 26000 and identified at nucleotide positions 61 to 62, and wherein the sensitivity is at least 45% and specificity is at least 78%.
535. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 26001 to 26500 and identified at nucleotide positions 61 to 62, and wherein the sensitivity is at least 52% and specificity is at least 76%.
536. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 26501 to 27000 and identified at nucleotide positions 61 to 62, and wherein the sensitivity is at least 49% and specificity is at least 74%.
537. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 27001 to 27500 and identified at nucleotide positions 61 to 62, and wherein the sensitivity is at least 49% and specificity is at least 72%.
538. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 27501 to 28000 and identified at nucleotide positions 61 to 62, and wherein the sensitivity is at least 40% and specificity is at least 76%.
539. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 28001 to 28500 and identified at nucleotide positions 61 to 62, and wherein the sensitivity is at least 43% and specificity is at least 76%.
540. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 28501 to 29000 and identified at nucleotide positions 61 to 62, and wherein the sensitivity is at least 43% and specificity is at least 76%.
541. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 29001 to 29500 and identified at nucleotide positions 61 to 62, and wherein the sensitivity is at least 42% and specificity is at least 76%.
542. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 29501 to 30000 and identified at nucleotide positions 61 to 62, and wherein the sensitivity is at least 47% and specificity is at least 74%.
543. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 30001 to 300500 and identified at nucleotide positions 61 to 62, and wherein the sensitivity is at least 44% and specificity is at least 74%.
544. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 30501 to 31000 and identified at nucleotide positions 61 to 62, and wherein the sensitivity is at least 46% and specificity is at least 76%.
545. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 31001 to 31500 and identified at nucleotide positions 61 to 62, and wherein the sensitivity is at least 43% and specificity is at least 77%.
546. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 31501 to 32000 and identified at nucleotide positions 61 to 62, and wherein the sensitivity is at least 47% and specificity is at least 76%.
547. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 32001 to 32500 and identified at nucleotide positions 61 to 62, and wherein the sensitivity is at least 44% and specificity is at least 76%.
548. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 32501 to 33000 and identified at nucleotide positions 61 to 62, and wherein the sensitivity is at least 48% and specificity is at least 76%.
549. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 33001 to 33500 and identified at nucleotide positions 61 to 62, and wherein the sensitivity is at least 42% and specificity is at least 76%.
550. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 33501 to 34000 and identified at nucleotide positions 61 to 62, and wherein the sensitivity is at least 36% and specificity is at least 77%.
551. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 34001 to 34500 and identified at nucleotide positions 61 to 62, and wherein the sensitivity is at least 38% and specificity is at least 77%.
552. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 34501 to 35000 and identified at nucleotide positions 61 to 62, and wherein the sensitivity is at least 41% and specificity is at least 76%.
553. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 35001 to 35500 and identified at nucleotide positions 61 to 62, and wherein the sensitivity is at least 40% and specificity is at least 76%.
554. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 35501 to 36000 and identified at nucleotide positions 61 to 62, and wherein the sensitivity is at least 40% and specificity is at least 74%.
555. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 36001 to 36500 and identified at nucleotide positions 61 to 62, and wherein the sensitivity is at least 38% and specificity is at least 77%.
556. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 36501 to 37000 and identified at nucleotide positions 61 to 62, and wherein the sensitivity is at least 38% and specificity is at least 78%.
557. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 37001 to 37500 and identified at nucleotide positions 61 to 62, and wherein the sensitivity is at least 39% and specificity is at least 77%.
558. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 37501 to 38000 and identified at nucleotide positions 61 to 62, and wherein the sensitivity is at least 39% and specificity is at least 79%.
559. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 38001 to 38500 and identified at nucleotide positions 61 to 62, and wherein the sensitivity is at least 40% and specificity is at least 75%.
560. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 38501 to 39000 and identified at nucleotide positions 61 to 62, and wherein the sensitivity is at least 42% and specificity is at least 74%.
561. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 39001 to 39500 and identified at nucleotide positions 61 to 62, and wherein the sensitivity is at least 35% and specificity is at least 76%.
562. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 39501 to 40000 and identified at nucleotide positions 61 to 62, and wherein the sensitivity is at least 40% and specificity is at least 73%.
563. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 40001 to 40500 and identified at nucleotide positions 61 to 62, and wherein the sensitivity is at least 35% and specificity is at least 76%.
564. An assay according to the invention, wherein the WID-BC-Index for the individual is about 0.090 or more, the individual is classified as having at least a moderate risk of harbouring breast cancer or a moderate risk of breast cancer development, wherein the set of CpGs comprises at least the CpGs defined by SEQ ID NOs 40501 to 40753 and identified at nucleotide positions 61 to 62, and wherein the sensitivity is at least 37% and specificity is at least 77%.
565. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1 to 500 and identified at nucleotide positions 61 to 62, and wherein the sensitivity is at least 26% and specificity is at least 93%.
566. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 501 to 1000 and identified at nucleotide positions 61 to 62, and wherein the sensitivity is at least 35% and specificity is at least 90%.
567. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1001 to 1500 and identified at nucleotide positions 61 to 62, and wherein the sensitivity is at least 39% and specificity is at least 90%.
568. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 1501 to 2000 and identified at nucleotide positions 61 to 62, and wherein the sensitivity is at least 34% and specificity is at least 91%.
569. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 2001 to 2500 and identified at nucleotide positions 61 to 62, and wherein the sensitivity is at least 34% and specificity is at least 89%.
570. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 2501 to 3000 and identified at nucleotide positions 61 to 62, and wherein the sensitivity is at least 33% and specificity is at least 87%.
571. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 3001 to 3500 and identified at nucleotide positions 61 to 62, and wherein the sensitivity is at least 46% and specificity is at least 89%.
572. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 3501 to 4000 and identified at nucleotide positions 61 to 62, and wherein the sensitivity is at least 37% and specificity is at least 89%.
573. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 4001 to 4500 and identified at nucleotide positions 61 to 62, and wherein the sensitivity is at least 46% and specificity is at least 84%.
574. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 4501 to 5000 and identified at nucleotide positions 61 to 62, and wherein the sensitivity is at least 39% and specificity is at least 89%.
575. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 5001 to 5500 and identified at nucleotide positions 61 to 62, and wherein the sensitivity is at least 39% and specificity is at least 86%.
576. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 5501 to 6000 and identified at nucleotide positions 61 to 62, and wherein the sensitivity is at least 32% and specificity is at least 91%.
577. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 6001 to 6500 and identified at nucleotide positions 61 to 62, and wherein the sensitivity is at least 47% and specificity is at least 88%.
578. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 6501 to 7000 and identified at nucleotide positions 61 to 62, and wherein the sensitivity is at least 46% and specificity is at least 87%.
579. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 7001 to 7500 and identified at nucleotide positions 61 to 62, and wherein the sensitivity is at least 27% and specificity is at least 87%.
580. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 7501 to 8000 and identified at nucleotide positions 61 to 62, and wherein the sensitivity is at least 48% and specificity is at least 87%.
581. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 8001 to 8500 and identified at nucleotide positions 61 to 62, and wherein the sensitivity is at least 29% and specificity is at least 87%.
582. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 8501 to 9000 and identified at nucleotide positions 61 to 62, and wherein the sensitivity is at least 37% and specificity is at least 88%.
583. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 9001 to 9500 and identified at nucleotide positions 61 to 62, and wherein the sensitivity is at least 38% and specificity is at least 84%.
584. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 9501 to 10000 and identified at nucleotide positions 61 to 62, and wherein the sensitivity is at least 28% and specificity is at least 88%.
585. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 10001 to 10500 and identified at nucleotide positions 61 to 62, and wherein the sensitivity is at least 31% and specificity is at least 87%.
586. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 10501 to 11000 and identified at nucleotide positions 61 to 62, and wherein the sensitivity is at least 33% and specificity is at least 92%.
587. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 11001 to 11500 and identified at nucleotide positions 61 to 62, and wherein the sensitivity is at least 54% and specificity is at least 86%.
588. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 11501 to 12000 and identified at nucleotide positions 61 to 62, and wherein the sensitivity is at least 41% and specificity is at least 81%.
589. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 12001 to 12500 and identified at nucleotide positions 61 to 62, and wherein the sensitivity is at least 39% and specificity is at least 87%.
590. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 12501 to 13000 and identified at nucleotide positions 61 to 62, and wherein the sensitivity is at least 33% and specificity is at least 89%.
591. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 13001 to 13500 and identified at nucleotide positions 61 to 62, and wherein the sensitivity is at least 45% and specificity is at least 87%.
592. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 13501 to 14000 and identified at nucleotide positions 61 to 62, and wherein the sensitivity is at least 45% and specificity is at least 87%.
593. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 14001 to 14500 and identified at nucleotide positions 61 to 62, and wherein the sensitivity is at least 42% and specificity is at least 87%.
594. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 14501 to 15000 and identified at nucleotide positions 61 to 62, and wherein the sensitivity is at least 39% and specificity is at least 85%.
595. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 15001 to 15500 and identified at nucleotide positions 61 to 62, and wherein the sensitivity is at least 39% and specificity is at least 86%.
596. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 15501 to 16000 and identified at nucleotide positions 61 to 62, and wherein the sensitivity is at least 41% and specificity is at least 85%.
597. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 16001 to 16500 and identified at nucleotide positions 61 to 62, and wherein the sensitivity is at least 42% and specificity is at least 86%.
598. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 16501 to 17000 and identified at nucleotide positions 61 to 62, and wherein the sensitivity is at least 32% and specificity is at least 88%.
599. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 17001 to 17500 and identified at nucleotide positions 61 to 62, and wherein the sensitivity is at least 38% and specificity is at least 84%.
600. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 17501 to 18000 and identified at nucleotide positions 61 to 62, and wherein the sensitivity is at least 33% and specificity is at least 86%.
601. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 18001 to 18500 and identified at nucleotide positions 61 to 62, and wherein the sensitivity is at least 35% and specificity is at least 86%.
602. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 18501 to 19000 and identified at nucleotide positions 61 to 62, and wherein the sensitivity is at least 31% and specificity is at least 86%.
603. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 19001 to 19500 and identified at nucleotide positions 61 to 62, and wherein the sensitivity is at least 33% and specificity is at least 86%.
604. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 19501 to 20000 and identified at nucleotide positions 61 to 62, and wherein the sensitivity is at least 31% and specificity is at least 86%.
605. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 20001 to 20500 and identified at nucleotide positions 61 to 62, and wherein the sensitivity is at least 36% and specificity is at least 86%.
606. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 20501 to 21000 and identified at nucleotide positions 61 to 62, and wherein the sensitivity is at least 34% and specificity is at least 85%.
607. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 21001 to 21500 and identified at nucleotide positions 61 to 62, and wherein the sensitivity is at least 29% and specificity is at least 88%.
608. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 21501 to 22000 and identified at nucleotide positions 61 to 62, and wherein the sensitivity is at least 28% and specificity is at least 87%.
609. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 22001 to 22500 and identified at nucleotide positions 61 to 62, and wherein the sensitivity is at least 27% and specificity is at least 86%.
610. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 22501 to 23000 and identified at nucleotide positions 61 to 62, and wherein the sensitivity is at least 33% and specificity is at least 87%.
611. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 23001 to 23500 and identified at nucleotide positions 61 to 62, and wherein the sensitivity is at least 31% and specificity is at least 85%.
612. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 23501 to 24000 and identified at nucleotide positions 61 to 62, and wherein the sensitivity is at least 31% and specificity is at least 86%.
613. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 24001 to 24500 and identified at nucleotide positions 61 to 62, and wherein the sensitivity is at least 36% and specificity is at least 86.
614. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 24501 to 25000 and identified at nucleotide positions 61 to 62, and wherein the sensitivity is at least 26% and specificity is at least 87%.
615. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 25001 to 25500 and identified at nucleotide positions 61 to 62, and wherein the sensitivity is at least 26% and specificity is at least 87%.
616. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 25501 to 26000 and identified at nucleotide positions 61 to 62, and wherein the sensitivity is at least 25% and specificity is at least 86%.
617. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 26001 to 26500 and identified at nucleotide positions 61 to 62, and wherein the sensitivity is at least 27% and specificity is at least 87%.
618. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 26501 to 27000 and identified at nucleotide positions 61 to 62, and wherein the sensitivity is at least 26% and specificity is at least 86%.
619. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 27001 to 27500 and identified at nucleotide positions 61 to 62, and wherein the sensitivity is at least 29% and specificity is at least 87%.
620. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 27501 to 28000 and identified at nucleotide positions 61 to 62, and wherein the sensitivity is at least 25% and specificity is at least 87%.
621. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 28001 to 28500 and identified at nucleotide positions 61 to 62, and wherein the sensitivity is at least 25% and specificity is at least 87%.
622. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 28501 to 29000 and identified at nucleotide positions 61 to 62, and wherein the sensitivity is at least 26% and specificity is at least 86%.
623. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 29001 to 29500 and identified at nucleotide positions 61 to 62, and wherein the sensitivity is at least 24% and specificity is at least 86%.
624. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 29501 to 30000 and identified at nucleotide positions 61 to 62, and wherein the sensitivity is at least 27% and specificity is at least 87%.
625. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 30001 to 30500 and identified at nucleotide positions 61 to 62, and wherein the sensitivity is at least 25% and specificity is at least 87%.
626. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 30501 to 31000 and identified at nucleotide positions 61 to 62, and wherein the sensitivity is at least 25% and specificity is at least 87%.
627. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 31001 to 31500 and identified at nucleotide positions 61 to 62, and wherein the sensitivity is at least 26% and specificity is at least 88%.
628. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 31501 to 32000 and identified at nucleotide positions 61 to 62, and wherein the sensitivity is at least 27% and specificity is at least 87%.
629. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 32001 to 32500 and identified at nucleotide positions 61 to 62, and wherein the sensitivity is at least 27% and specificity is at least 86%.
630. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 32501 to 33000 and identified at nucleotide positions 61 to 62, and wherein the sensitivity is at least 28% and specificity is at least 86%.
631. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 33001 to 33500 and identified at nucleotide positions 61 to 62, and wherein the sensitivity is at least 25% and specificity is at least 88%.
632. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 33501 to 34000 and identified at nucleotide positions 61 to 62, and wherein the sensitivity is at least 26% and specificity is at least 89%.
633. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 34001 to 34500 and identified at nucleotide positions 61 to 62, and wherein the sensitivity is at least 24% and specificity is at least 88%.
634. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 34501 to 35000 and identified at nucleotide positions 61 to 62, and wherein the sensitivity is at least 26% and specificity is at least 88%.
635. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 35001 to 35500 and identified at nucleotide positions 61 to 62, and wherein the sensitivity is at least 26% and specificity is at least 87%.
636. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 35501 to 36000 and identified at nucleotide positions 61 to 62, and wherein the sensitivity is at least 26% and specificity is at least 86%.
637. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 36001 to 36500 and identified at nucleotide positions 61 to 62, and wherein the sensitivity is at least 24% and specificity is at least 86%.
638. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 36501 to 37000 and identified at nucleotide positions 61 to 62, and wherein the sensitivity is at least 27% and specificity is at least 88%.
639. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 37001 to 37500 and identified at nucleotide positions 61 to 62, and wherein the sensitivity is at least 26% and specificity is at least 87%.
640. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 37501 to 38000 and identified at nucleotide positions 61 to 62, and wherein the sensitivity is at least 24% and specificity is at least 87%.
641. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 38001 to 38500 and identified at nucleotide positions 61 to 62, and wherein the sensitivity is at least 27% and specificity is at least 87%.
642. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 38501 to 39000 and identified at nucleotide positions 61 to 62, and wherein the sensitivity is at least 26% and specificity is at least 87%.
643. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 39001 to 39500 and identified at nucleotide positions 61 to 62, and wherein the sensitivity is at least 25% and specificity is at least 87%.
644. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 39501 to 40000 and identified at nucleotide positions 61 to 62, and wherein the sensitivity is at least 25% and specificity is at least 87%.
645. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 40001 to 40500 and identified at nucleotide positions 61 to 62, and wherein the sensitivity is at least 25% and specificity is at least 87%.
646. An assay according to the invention, wherein the WID-BC-Index for the individual is about 0.587 or more, the individual is classified as having at least a high risk of harbouring breast cancer or a high risk of breast cancer development, wherein the set of CpGs comprises at least the CpGs defined by SEQ ID NOs 40501 to 40753 and identified at nucleotide positions 61 to 62, and wherein the sensitivity is at least 24% and specificity is at least 87%.

### MAIN REFERENCES

(denoted in main text by superscript numbering)
1 Harbeck, N. & Gnant, M. Breast cancer. Lancet 389, 1134-1150, doi:10.1016/S0140-6736(16)31891-8 (2017).
2 Warner, E. Clinical practice. Breast-cancer screening. N. Engl. J. Med 365, 1025-1032, doi:10.1056/NEJMcp1101540 [doi] (2011).
3 Shen, S. Y. et al. Sensitive tumour detection and classification using plasma cell-free DNA methylomes. Nature 563, 579-583, doi:10.1038/s41586-018-0703-0 (2018).
4 Widschwendter, M. et al. Methylation patterns in serum DNA for early identification of disseminated breast cancer. Genome Med 9, 115, doi:10.1186/s13073-017-0499-9 (2017).
5 Autier, P. & Boniol, M. Mammography screening: A major issue in medicine. Eur J Cancer 90, 34-62, doi:10.1016/j.ejca.2017.11.002 (2018).
6 Gail, M. H. & Pfeiffer, R. M. Breast Cancer Risk Model Requirements for Counseling, Prevention, and Screening. J Natl Cancer Inst 110, 994-1002, doi:10.1093/jnci/djy013 (2018).
7 Cohen, P. A., Jhingran, A., Oaknin, A. & Denny, L. Cervical cancer. Lancet 393, 169-182, doi:10.1016/50140-6736(18)32470-X (2019).
8 Polman, N. J. et al. Performance of human papillomavirus testing on self-collected versus clinician-collected samples for the detection of cervical intraepithelial neoplasia of grade 2 or worse: a randomised, paired screen-positive, non-inferiority trial. Lancet Oncol 20, 229-238, doi:10.1016/S1470-2045(18)30763-0 (2019).
9 Teschendorff, A. E. et al. DNA methylation outliers in normal breast tissue identify field defects that are enriched in cancer. Nat. Commun 7, 10478, doi:ncomms10478 [pii]; 10.1038/ncomms10478 [doi] (2016).
10 Lichtenstein, P. et al. Environmental and heritable factors in the causation of cancer—analyses of cohorts of twins from Sweden, Denmark, and Finland. N. Engl. J. Med 343, 78-85 (2000).
11 Ambatipudi, S. et al. DNA methylome analysis identifies accelerated epigenetic ageing associated with postmenopausal breast cancer susceptibility. Eur J Cancer 75, 299-307, doi:10.1016/j.ejca.2017.01.014 (2017).
12 Anjum, S. et al. A BRCA1-mutation associated DNA methylation signature in blood cells predicts sporadic breast cancer incidence and survival. Genome Med 6, 47, doi:10.1186/gm567 [doi]; gm567 [pii] (2014).
13 Severi, G. et al. Epigenome-wide methylation in DNA from peripheral blood as a marker of risk for breast cancer. Breast Cancer Res Treat 148, 665-673, doi:10.1007/s10549-014-3209-y (2014).
14 van Veldhoven, K. et al. Epigenome-wide association study reveals decreased average methylation levels years before breast cancer diagnosis. Clin Epigenetics 7, 67, doi:10.1186/s13148-015-0104-2 (2015).
15 Xu, Z. et al. Epigenome-wide association study of breast cancer using prospectively collected sister study samples. J Natl. Cancer Inst 105, 694-700, doi:djt045 [pii]; 10.1093/jnci/djt045 [doi] (2013).
16 Brennan, K. et al. Intragenic ATM methylation in peripheral blood DNA as a biomarker of breast cancer risk. Cancer Res, doi:0008-5472.CAN-11-3157 [pii]; 10.1158/0008-5472.CAN-11-3157 [doi] (2012).
17 Widschwendter, M. et al. Epigenome-based cancer risk prediction: rationale, opportunities and challenges. Nat Rev Clin Oncol, doi:10.1038/nrclinonc.2018.30 (2018).
18 Pidsley, R. et al. Critical evaluation of the Illumina MethylationEPIC BeadChip microarray for whole-genome DNA methylation profiling. Genome Biol 17, 208, doi:10.1186/s13059-016-1066-1 (2016).
19 Zheng, S. C. et al. A novel cell-type deconvolution algorithm reveals substantial contamination by immune cells in saliva, buccal and cervix. Epigenomics, doi:10.2217/epi-2018-0037 (2018).
20 Breeze, C. E. et al. eFORGE: A Tool for Identifying Cell Type-Specific Signal in Epigenomic Data. Cell Rep 17, 2137-2150, doi:10.1016/j.celrep.2016.10.059 (2016).
21 Subramanian, A. et al. Gene set enrichment analysis: a knowledge-based approach for interpreting genome-wide expression profiles. Proc. Natl. Acad. Sci. U. S. A 102, 15545-15550 (2005).
22 Mavaddat, N. et al. Polygenic Risk Scores for Prediction of Breast Cancer and Breast Cancer Subtypes. Am J Hum Genet 104, 21-34, doi:10.1016/j.ajhg.2018.11.002 (2019).
23 Feinberg, A. P., Koldobskiy, M. A. & Gondor, A. Epigenetic modulators, modifiers and mediators in cancer aetiology and progression. Nat Rev Genet 17, 284-299, doi:10.1038/nrg.2016.13 (2016).
24 Salhab, A. et al. A comprehensive analysis of 195 DNA methylomes reveals shared and cell-specific features of partially methylated domains. Genome Biol 19, 150, doi:10.1186/s13059-018-1510-5 (2018).
25 Rivenbark, A. G. & Coleman, W. B. Field cancerization in mammary carcinogenesis—Implications for prevention and treatment of breast cancer. Exp Mot Pathol 93, 391-398, doi:10.1016/j.yexmp.2012.10.018 (2012).
26 Haggie, J. A., Sellwood, R. A., Howell, A., Birch, J. M. & Schor, S. L. Fibroblasts from relatives of patients with hereditary breast cancer show fetal-like behaviour in vitro. Lancet 1, 1455-1457 (1987).
27 Purcell, S. et al. PLINK: a tool set for whole-genome association and population-based linkage analyses. Am J Hum Genet 81, 559-575, doi:10.1086/519795 (2007).
28 Manichaikul, A. et al. Robust relationship inference in genome-wide association studies. Bioinformatics 26, 2867-2873, doi:10.1093/bioinformatics/btq559 (2010).
29 Das, S. et al. Next-generation genotype imputation service and methods. Nat Genet 48, 1284-1287, doi:10.1038/ng.3656 (2016).
30 Michailidou, K. et al. Association analysis identifies 65 new breast cancer risk loci. Nature 551, 92-94, doi:10.1038/nature24284 (2017).

### SUPPLEMENTARY REFERENCES

(denoted in main text by double square brackets [[ ]])
[[1]]. Olkhov-Mitsel, E and Bapat, B: Strategies for discovery and validation of methylated and hydroxymethylated DNA biomarkers. Cancer Medicine 2012, 1(2): 237-260.
[[2]]. Herman, J. G., Graff, J. R., Myohanen, S., Nelkin, B. D. & Baylin, S. B.: Methylation-specific PCR: a novel PCR assay for methylation status of CpG islands. Proc. Natl Acad. Sci. USA 1996, 93: 9821-9826.
[[3]]. Frommer, M. et al.: A genomic sequencing protocol that yields a positive display of 5-methylcytosine residues in individual DNA strands. Proc. Natl Acad. Sci. USA 1992, 89: 1827-1831.
[[4]]. Xiong, Z. & Laird, P. W.: COBRA: a sensitive and quantitative DNA methylation assay. Nucleic Acids Res. 1997, 25: 2532-2534.
[[5]]. Gonzalgo, M. L. & Jones, P. A.: Rapid quantitation of methylation differences at specific sites using methylationsensitive single nucleotide primer extension (Ms-SNuPE). Nucleic Acids Res. 1997, 25: 2529-2531.
[[6]]. Paul D S, Guilhamon P, Karpathakis A, Butcher L M, Thirlwell C, Feber A, Beck S: Assessment of RainDrop BS-seq as a method for large-scale, targeted bisulfite sequencing. Epigenetics 2014, 9.
[[7]]. Singal, R. & Grimes, S. R.: Microsoft Word macro for analysis of cytosine methylation by the bisulfite deamination reaction. Biotechniques 2001, 30: 116-120.
[[8]]. Anbazhagan, R., Herman, J. G., Enika, K. & Gabrielson, E.: Spreadsheet-based program for the analysis of DNA methylation. Biotechniques 2001, 30: 110-114.
[[9]]. Li, L. C. & Dahiya, R.: MethPrimer: designing primers for methylation PCRs. Bioinformatics 2002, 18: 1427-1431.
[[10]]. Eng, J: Receiver Operating Characteristic Analysis: A Primer. Academic Radiology 2005, 12(7): 909-916.

1. An assay for predicting the presence or development of breast cancer in an individual, the assay comprising:
a. providing a sample which has been taken from the individual;
b. determining in DNA in the sample the methylation status of each CpG in a set of test CpGs selected from the panel of CpGs identified at nucleotide positions 61 to 62 in SEQ ID NOs 1 to 40,753;
c. deriving a breast cancer index value based on the methylation status of the test CpGs; and
d. predicting the presence or development of breast cancer in the individual based on the breast cancer index value;
wherein the assay is characterised as having an area under the curve (AUC) of 0.6 or more as determined by receiver operating characteristics (ROC).

2. An assay according to claim 1, wherein the sample from the individual is a sample from:
a. an anatomical site other than the breast, such as a cervical, vaginal, or preferably a cervicovaginal smear; or
b. the breast.

3. An assay according to claim 1 or 2, wherein the DNA from the sample is derived from an organ comprising epithelial cells.

4. An assay according to any one of claims 1 to 3, wherein the cancer is ductal carcinoma in situ; an invasive ductal carcinoma such as tubular type invasive ductal carcinoma (IDC), medullary type IDC, mucinous type IDC, papillary type IDC, cribriform type IDC; invasive lobular carcinoma, inflammatory breast cancer, lobular carcinoma in situ, male breast cancer, luminal A breast cancer, luminal B breast cancer, triple-negative/basal-like breast cancer, HER2-enriched breast cancer, normal-like breast cancer, Paget's Disease of the nipple, Phyllodes tumours of the breast, or metastatic breast cancer.

5. An assay according to any one of claims 1 to 4, wherein the step of determining the methylation status of each CpG in the set of test CpGs comprises determining methylation β-values for each one of the test CpGs.

6. An assay according to claim 5, wherein the step of deriving the breast cancer index value based on the methylation status of the test CpGs comprises:
a. providing a methylation β-value data set comprising the methylation β-values for each test CpG;
b. estimating an immune cell DNA fraction within the DNA provided from the sample; and
c. applying an algorithm to the methylation β-value data set that controls for the immune cell DNA fraction within the DNA provided from the sample to obtain the breast cancer index value.

7. An assay according to claim 6, wherein the breast cancer index value is termed Women's risk Identification for Breast Cancer Index (WID-BC-Index) and is calculated by an algorithm according to the following formula:
$$WID - BC - Index = {\sum }_{i = 1}^{n} \frac{μ - ( {a}_{i} + {b}_{i} ρ ) {β}_{i}}{σ}$$
wherein:
a. ρ∈[0,1] is the immune cell DNA fraction for the sample;
b. β₁, . . . , βₙ are methylation beta-values (between 0 and 1);
c. a₁, . . . , aₙ and b₁, bₙ are real valued coefficients;
d. μ and σ are real valued parameters used to scale the index; and
e. n refers to the number of CpGs in the set of test CpGs

8. An assay according to any one of claims 1 to 7, wherein the set of CpGs comprises at least 500 CpGs selected from the CpGs identified at nucleotide positions 61 to 62 in SEQ ID NOs 1 to 40,753, preferably wherein the assay is characterised as having an AUC of at least 0.73.

9. An assay according to claim 8, wherein the set of CpGs comprises at least the CpGs identified in SEQ ID NOs 1 to 500 and identified at nucleotide positions 61 to 62.

10. An assay according to any one of claims 1 to 7, wherein the set of CpGs comprises at least 1000 CpGs selected from the CpGs identified at nucleotide positions 61 to 62 in SEQ ID NOs 1 to 40,753, preferably wherein the assay is characterised as having an AUC of at least 0.77.

11. An assay according to claim 10, wherein the set of CpGs comprises at least the CpGs identified in SEQ ID NOs 1 to 1000 and identified at nucleotide positions 61 to 62.

12. An assay according to any one of claims 1 to 7, wherein the set of CpGs comprises at least 2000 CpGs selected from the CpGs identified at nucleotide positions 61 to 62 in SEQ ID NOs 1 to 40,753, preferably wherein the assay is characterised as having an AUC of at least 0.81.

13. An assay according to claim 12, wherein the set of CpGs comprises at least the CpGs identified in SEQ ID NOs 1 to 2000 and identified at nucleotide positions 61 to 62.

14. An assay according to any one of claims 1 to 7, wherein the set of CpGs comprises at least 10,000 CpGs selected from the CpGs identified at nucleotide positions 61 to 62 in SEQ ID NOs 1 to 40,753, preferably wherein the assay is characterised as having an AUC of at least 0.84.

15. An assay according to claim 14, wherein the set of CpGs comprises at least the CpGs identified in SEQ ID NOs 1 to 10,000 and identified at nucleotide positions 61 to 62.

16. An assay according to any one of claims 1 to 7, wherein the set of CpGs comprises at least the 40,753 CpGs identified in SEQ ID NOs 1 to 40,753 and identified at nucleotide positions 61 to 62, and further wherein the assay is characterised as having an AUC of at least 0.85.

17. An assay according to any one of claims 7 to 16, wherein the predicting of the presence or development of breast cancer in an individual is based on the WID-BC-Index.

18. An assay according to claim 17, wherein when the WID-BC-Index for the individual is about −0.235 or more, the individual is classified as having at least a low risk of harbouring breast cancer or a low risk of breast cancer development.

19. An assay according to claim 17, wherein when the WID-BC-Index for the individual is about −0.235 or more, the individual is classified as harbouring breast cancer, or wherein when the WID-BC-Index for the individual is less than about −0.235 the individual is classified as not harbouring breast cancer.

20. An assay according to claim 18 or 19, wherein the set of CpGs comprises at least 500 of the CpGs defined by SEQ ID NOs 1 to 40,753 and identified at nucleotide positions 61 to 62, and wherein the sensitivity is at least 58% and the specificity is at least 44%.

21. An assay according to claim 18 or 19, wherein the set of CpGs comprises at least the CpGs defined by SEQ ID NOs 1 to 500 and identified at nucleotide positions 61 to 62, and wherein the sensitivity is at least 85% and specificity is at least 52%.

22. An assay according to claim 18 or 19, wherein the set of CpGs comprises at least the CpGs defined by SEQ ID NOs 1 to 1000 and identified at nucleotide positions 61 to 62, and wherein the sensitivity is at least 88% and specificity is at least 49%.

23. An assay according to claim 18 or 19, wherein the set of CpGs comprises at least the CpGs defined by SEQ ID NOs 1 to 2000 and identified at nucleotide positions 61 to 62, and wherein the sensitivity is at least 94% and specificity is at least 51%.

24. An assay according to claim 17, wherein when the WID-BC-Index for the individual is about 0.090 or more, the individual is classified as having at least a moderate risk of harbouring breast cancer or a moderate risk of breast cancer development.

25. An assay according to claim 17, wherein when the WID-BC-Index for the individual is about 0.090 or more, the individual is classified as harbouring breast cancer, or wherein when the WID-BC-Index for the individual is less than about 0.090 the individual is classified as not harbouring breast cancer.

26. An assay according to claim 24 or 25, wherein the set of CpGs comprises at least 500 of the CpGs defined by SEQ ID NOs 1 to 40,753 and identified at nucleotide positions 61 to 62, and wherein the sensitivity is at least 35% and the specificity is at least 63%.

27. An assay according to claim 24 or 25, wherein the set of CpGs comprises at least the CpGs defined by SEQ ID NOs 1 to 500 and identified at nucleotide positions 61 to 62, and wherein the sensitivity is at least 63% and specificity is at least 69%.

28. An assay according to claim 24 or 25, wherein the set of CpGs comprises at least the CpGs defined by SEQ ID NOs 1 to 1000 and identified at nucleotide positions 61 to 62, and wherein the sensitivity is at least 68% and specificity is at least 73%.

29. An assay according to claim 24 or 25, wherein the set of CpGs comprises at least the CpGs defined by SEQ ID NOs 1 to 2000 and identified at nucleotide positions 61 to 62, and wherein the sensitivity is at least 69% and specificity is at least 78%.

30. An assay according to claim 17, wherein when the WID-BC-Index for the individual is about 0.587 or more, the individual is classified as having a high risk of harbouring breast cancer or a high risk of breast cancer development.

31. An assay according to claim 17, wherein when the WID-BC-Index for the individual is about 0.587 or more, the individual is classified as harbouring breast cancer, or wherein when the WID-BC-Index for the individual is less than about 0.587 the individual is classified as not harbouring breast cancer.

32. An assay according to claim 30 or 31, wherein the set of CpGs comprises at least 500 of the CpGs defined by SEQ ID NOs 1 to 40,753 and identified at nucleotide positions 61 to 62, and wherein the sensitivity is at least 24% and the specificity is at least 84%.

33. An assay according to claim 30 or 31, wherein the set of CpGs comprises at least the CpGs defined by SEQ ID NOs 1 to 500 and identified at nucleotide positions 61 to 62, and wherein the sensitivity is at least 26% and specificity is at least 93%.

34. An assay according to claim 30 or 31, wherein the set of CpGs comprises at least the CpGs defined by SEQ ID NOs 1 to 1000 and identified at nucleotide positions 61 to 62, and wherein the sensitivity is at least 29% and specificity is at least 95%.

35. An assay according to claim 30 or 31, wherein the set of CpGs comprises at least the CpGs defined by SEQ ID NOs 1 to 2000 and identified at nucleotide positions 61 to 62, and wherein the sensitivity is at least 33% and specificity is at least 94%.

36. An assay according to any one of claims 1 to 35, wherein the step of determining the methylation status of each CpG in the set of test CpGs comprises bisulphite converting the DNA.

37. An assay according to any one of claims 1 to 36, wherein the step of determining the methylation status of each CpG in the set of test CpGs comprises:
a. performing a sequencing step to determine the sequence of each CpG;
b. hybridising DNA to an array comprising probes capable of discriminating between methylated and non-methylated forms of the CpGs and applying a detection system to the array so as to determine the methylation status or β-value of each CpG; or
c. performing a PCR step using methylation-specific primers, wherein the methylation status of the CpG is determined by the presence or absence of a PCR product.

38. An assay according to any one of claims 1 to 37, wherein the assay further comprises:
a. determining in the sample from the individual the proportion of epithelial cells;
b. determining in the sample from the individual the proportion of fat cells; and/or
c. determining in the sample from the individual differentiation characteristics of non-fat cells.

39. An assay according to claim 38, wherein determining the proportion of epithelial and/or fat cells and/or determining differentiation characteristics of non-fat cells comprises performing a method comprising:
a. gene expression profiling;
b. non-coding RNA-profiling;
c. epigenome profiling;
d. DNA methylation profiling;
e. deriving a WID-BC-Index; and/or
f. immunohistochemistry; and
arriving at a determination based on the results of the method.

40. An array capable of discriminating between methylated and non-methylated forms of CpGs; the array comprising oligonucleotide probes specific for a methylated form of each CpG in a CpG panel and oligonucleotide probes specific for a non-methylated form of each CpG in the panel; wherein the panel consists of at least 500 CpGs selected from the CpGs identified in SEQ ID NOs 1 to 40,753.

41. An array according to claim 40, provided that the array is not an Infinium MethylationEPIC BeadChip array or an Infinium HumanMethylation450, and/or provided that the number of CpG-specific oligonucleotide probes of the array is 482,000 or less, 480,000 or less, 450,000 or less, 440,000 or less, 430,000 or less, 420,000 or less, 410,000 or less, or 400,000 or less.

42. An array according to claim 40 or 41, wherein the panel comprises any set of CpGs defined in the assays of any one of claims 8 to 16.

43. An array according to any one of claims 40 to 42, further comprising one or more oligonucleotides comprising any set of CpGs defined in the assays of any one of claims 8 to 16, wherein the one or more oligonucleotides are hybridized to corresponding oligonucleotide probes of the array.

44. A hybridized array, wherein the array is obtainable by hybridizing to an array according to any one of claims 40 to 43 a group of oligonucleotides comprising any set of CpGs defined in the assays of any one of claims 8 to 16.

45. A process for making the hybridized array according to claim 44, comprising contacting an array according to claims 40 to 43 with a group of oligonucleotides comprising any set of CpGs defined in the assays of any one of claims 8 to 16.

46. A method of treating breast cancer in an individual comprising:
a. predicting the presence or development of breast cancer in an individual by performing the assay of any one of claims 1 to 39;
b. stratifying the individual according to their risk of harbouring breast cancer or according to their risk of breast cancer development; and
c. administering one or more treatments to the individual based on their risk stratification or based on whether the individual is classified as harbouring breast cancer or not.

47. A method according to claim 46, wherein the individual is stratified as having a low risk of harbouring breast cancer or a low risk of breast cancer development and the individual is subjected to treatment according to their risk, e.g. intensified screening.

48. A method according to claim 47, wherein the intensified screening comprises one or more mammography scans and/or breast MRI scans.

49. A method according to claim 46, wherein the individual is stratified as having a moderate risk of harbouring breast cancer or a moderate risk of breast cancer development and the individual is subjected to treatment according to their risk, e.g. intensified screening and/or administration of one or more doses of one or more of Mifeprestone, Aromatase inhibitors, Denosumab, “selective estrogen modulators” (SERMs) and “selective progesterone receptor modulators” (SPRMs).

50. A method according to claim 49, wherein the intensified screening comprises one or more mammography scans and/or breast MRI scans.

51. A method according to claim 46, wherein the individual is stratified as having a high risk of harbouring breast cancer or a high risk of breast cancer development and the individual is subjected to treatment according to their risk, e.g.:
a. intensified screening, optionally comprising one or mammography scans and/or breast MRI scans;
b. administration of one or more of Mifeprestone, Aromatase inhibitors, Denosumab, SERMs and SPRMs; and/or
c. bilateral mastectomy.

52. A method according to claim 46, wherein when the individual is classified as harbouring breast cancer, the individual is subjected to any one or more of the treatments defined by claims 47 to 51.

53. A method of monitoring the risk of an individual harbouring breast cancer or monitoring the risk of breast cancer development, the method comprising: (a) predicting the presence of breast cancer in an individual or predicting breast cancer development in an individual by performing the assay according to any one of claims 1 to 36 at a first time point; (b) predicting the presence of breast cancer in the individual or predicting breast cancer development in the individual by performing the assay according to any one of claims 1 to 39 at one or more further time points; and (c) monitoring any change in the individual's risk between time points.

54. A method according to claim 53, wherein the further time points are three monthly, six monthly, yearly or two yearly basis following an initial prediction.

55. A method according to claim 53 or 54, wherein the individual does not harbour breast cancer and harbours one or more genetic mutations that predispose the individual to an increased risk of developing breast cancer.

56. A method according to claim 55, wherein the individual harbours one or more mutations in a BRCA gene.

57. A method according to claims 53 to 56, wherein depending on the risk of the presence of breast cancer in the individual or risk of breast cancer development in the individual, one or more treatments are administered to the individual according to any one of claims 46 to 52.

58. A method according to claim 57, wherein the individual has an increased risk of breast cancer development and wherein one or more treatments are administered to the individual according to any one of claims 43 to 48 as a method of prophylaxis.

59. A method according to claim 58, wherein the one or more treatments administered to the individual comprises one or more doses of SPRMs.

60. A method according to claim 59, wherein the one or more doses of SPRMs comprises one or more doses of Mifepristone.

61. A method according to any one of claims 53 to 60, wherein the method further comprises:
a. determining the proportion of epithelial cells in the sample from the individual between any two or more time points and assessing if the proportion changes between time points;
b. determining the proportion of fat cells in the sample from the individual between any two or more time points and assessing if the proportion changes between time points; and/or
c. determining differentiation characteristics of non-fat cells in the sample from the individual between any two or more time points and assessing if the proportion changes between time points.

62. A method according to any one of claims 53 to 61, wherein:
a. an increase in the breast cancer index value and an increase in the proportion of epithelial cells; and/or
b. an increase in the breast cancer index value and a decrease in the proportion of fat cells; and/or
c. an increase in the breast cancer index value and an increase in differentiation of non-fat cells towards fat cells,
indicates a negative response to the one or more treatments.

63. A method according to claim 59, wherein changes are made to the one or more treatments if a negative response is identified.

64. A method according to any one of claims 53 to 61, wherein:
a. a decrease in the breast cancer index value and a decrease in the proportion of epithelial cells;
b. a decrease in the breast cancer index value and an increase in the proportion of fat cells; and/or
c. a decrease in the breast cancer index value and a decrease in differentiation of non-fat cells towards fat cells,
indicates a positive response to the one or more treatments.

65. A method according to claim 64, wherein changes are made to the one or more treatments if a positive response is identified.
